SEQUENCE LISTING (94 sequences)

SEQ ID NO: 1 - DNA, 636 bp, Artificial
  anti-EGFR Fab light chain
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 2 - protein, 214 aa, Artificial
  anti-EGFR Fab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 666 bp, Artificial
  anti-EGFR Fab heavy chain
caggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 4 - protein, 222 aa, Artificial
  anti-EGFR Fab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - DNA, 15 bp, Artificial
  linker
ggtggaggtgggagc

SEQ ID NO: 6 - protein, 5 aa, Artificial
  linker
GGGGS

SEQ ID NO: 7 - DNA, 15 bp, Artificial
  mutated hinge region
gagcccaaatcttct

SEQ ID NO: 8 - protein, 5 aa, Artificial
  mutated hinge region
EPKSS

SEQ ID NO: 9 - DNA, 15 bp, Artificial
  hinge region
gagcccaaatcttgt

SEQ ID NO: 10 - protein, 5 aa, Artificial
  hinge region
EPKSC

SEQ ID NO: 11 - DNA, 1437 bp, Artificial
  H-CH2-CH3-L-VH(anti-EGFR)-CH1-H (L = G4S)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggtggaggtgggagccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttga

SEQ ID NO: 12 - DNA, 702 bp, Artificial
  VL(anti-EGFR)-CL
atggagttgcctgttaggctgttggtgctgatgttctggattcctgcttccttaagcgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 458 aa, Artificial
  H-CH2-CH3-L-VH(anti-EGFR)-CH1-H (L = G4S)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 14 - protein, 214 aa, Artificial; identical to SEQ ID NO: 2
  VL(anti-EGFR)-CL

SEQ ID NO: 15 - DNA, 1413 bp, Artificial
  H-CH2-CH3-L-VL(anti-EGFR)-CL (L = G4S)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggtggaggtgggagcgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 16 - DNA, 726 bp, Artificial
  VH(anti-EGFR)-CH1-H
atggagttgcctgttaggctgttggtgctgatgttctggattcctgcttccagcagccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgttag

SEQ ID NO: 17 - protein, 450 aa, Artificial
  H-CH2-CH3-L-VL(anti-EGFR)-CL (L = G4S)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 222 aa, Artificial; identical to SEQ ID NO: 4
  VH(anti-EGFR)-CH1-H

SEQ ID NO: 19 - DNA, 1461 bp, Artificial
  H-CH2-CH3-L-VL(anti-EGFR)-CL (L = (G4S)4)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggcggcggaggaagcggaggaggtggcagcggtggcggtggctccggcggaggtggctccggagacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 466 aa, Artificial
  H-CH2-CH3-L-VL(anti-EGFR)-CL (L = (G4S)4)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 633 bp, Artificial
  anti-CD20 Fab light chain
caaattgttctctcccagtctccagcaatcctgtctgcatctccaggggagaaggtcacaatgacttgcagggccagctcaagtgtaagttacatccactggttccagcagaagccaggttcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 22 - protein, 213 aa, Artificial
  anti-CD20 Fab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 669 bp, Artificial
  anti-CD20 Fab heavy chain
caggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatct

SEQ ID NO: 24 - protein, 224 aa, Artificial
  anti-CD20 Fab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - DNA, 1443 bp, Artificial
  H-CH2-CH3-L-VH(anti-CD20)-CH1-H (L = G4S)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggtggaggtgggagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttga

SEQ ID NO: 26 - DNA, 693 bp, Artificial
  VL(anti-CD20)-CL
atggagttgcctgttaggctgttggtgctgatgttctggattcctgcttccttaagccaaattgttctctcccagtctccagcaatcctgtctgcatctccaggggagaaggtcacaatgacttgcagggccagctcaagtgtaagttacatccactggttccagcagaagccaggttcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - protein, 461 aa, Artificial
  H-CH2-CH3-L-VH(anti-CD20)-CH1-H (L = G4S)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 214 aa, Artificial
  VL(anti-CD20)-CL
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 1491 bp, Artificial
  H-CH2-CH3-L-VH(anti-CD20)-CH1-H (L = (G4S)4)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggcggcggaggaagcggaggaggtggcagcggtggcggtggctccggcggaggtggctccggacaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttga

SEQ ID NO: 30 - protein, 477 aa, Artificial
  H-CH2-CH3-L-VH(anti-CD20)-CH1-H (L = (G4S)4)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - DNA, 1410 bp, Artificial
  H-CH2-CH3-L-VL(anti-CD20)-CL (L = G4S)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggtggaggtgggagccaaattgttctctcccagtctccagcaatcctgtctgcatctccaggggagaaggtcacaatgacttgcagggccagctcaagtgtaagttacatccactggttccagcagaagccaggttcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 32 - DNA, 732 bp, Artificial
  VH(anti-CD20)-CH1-H
atggagttgcctgttaggctgttggtgctgatgttctggattcctgcttccagcagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcgccgtgatcagcagcggcggcagctccatcaactacaagaaagttgagcccaaatcttgttaa

SEQ ID NO: 33 - protein, 449 aa, Artificial
  H-CH2-CH3-L-VL(anti-CD20)-CL (L = G4S)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 224 aa, Artificial; identical to SEQ ID NO: 24
  VH(anti-CD20)-CH1-H

SEQ ID NO: 35 - DNA, 1458 bp, Artificial
  H-CH2-CH3-L-VL(anti-CD20)-CL (L = (G4S)4)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggcggcggaggaagcggaggaggtggcagcggtggcggtggctccggcggaggtggctccggacaaattgttctctcccagtctccagcaatcctgtctgcatctccaggggagaaggtcacaatgacttgcagggccagctcaagtgtaagttacatccactggttccagcagaagccaggttcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 36 - protein, 465 aa, Artificial
  H-CH2-CH3-L-VL(anti-CD20)-CL (L = (G4S)4)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 648 bp, Artificial
  anti-CD16 Fab light chain
gacattgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctggcatcccagccaggtttagtgccagtgggtctgggacagacttcaccctcaacatccatcctgtggaggaggaggatactgcaacctattactgtcagcaaagtaatgaggacccgtacacgttcggaggggggaccaagctggagctgaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 38 - protein, 218 aa, Artificial
  anti-CD16 Fab light chain
DIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 672 bp, Artificial
  anti-CD16 Fab heavy chain
caggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 40 - protein, 221 aa, Artificial
  anti-CD16 Fab heavy chain
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - DNA, 2073 bp, Artificial
  VH(anti-EGFR)-CH1-H-CH2-CH3-L-VL(anti-CD16)-CL (L = G4S)
atggagttgcctgttaggctgttggtgctgatgttctggattcctgctagctccagccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctccagtggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatccagcatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacacagaagagcctctccctgtccccgggtgcaggtggaggtgggagcgacattgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctggcatcccagccaggtttagtgccagtgggtctgggacagacttcaccctcaacatccatcctgtggaggaggaggatactgcaacctattactgtcagcaaagtaatgaggacccgtacacgttcggaggggggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 42 - DNA, 732 bp, Artificial
  VH(anti-CD16)-CH1-H
atggagttgcctgttaggctgttggtgctgatgttctggattcctgcttccagcagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgttag

SEQ ID NO: 43 - protein, 671 aa, Artificial
  VH(anti-EGFR)-CH1-H-CH2-CH3-L-VL(anti-CD16)-CL (L = G4S)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 221 aa, Artificial
  VH(anti-CD16)-CH1-H
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 45 - DNA, 2067 bp, Artificial
  VH(anti-CD16)-CH1-H-CH2-CH3-L-VL(anti-EGFR)-CL (L = G4S)
atggagttgcctgttaggctgttggtgctgatgttctggattcctgctagctccagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctccagtggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatccagcatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacacagaagagcctctccctgtccccgggtgcaggtggaggtgggagcgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 46 - DNA, 714 bp, Artificial
  VL(anti-CD16)-CL
atggagttgcctgttaggctgttggtgctgatgttctggattcctgcttccttaagcgacattgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctggcatcccagccaggtttagtgccagtgggtctgggacagacttcaccctcaacatccatcctgtggaggaggaggatactgcaacctattactgtcagcaaagtaatgaggacccgtacacgttcggaggggggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 47 - protein, 666 aa, Artificial
  VH(anti-CD16)-CH1-H-CH2-CH3-L-VL(anti-EGFR)-CL (L = G4S)
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 218 aa, Artificial; identical to SEQ ID NO: 38
  VL(anti-CD16)-CL

SEQ ID NO: 49 - DNA, 2082 bp, Artificial
  VH(anti-CD20)-CH1-H-CH2-CH3-L-VL(anti-CD16)-CL (L = G4S)
atggagttgcctgttaggctgttggtgctgatgttctggattcctgctagctccagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgcaggtggaggtgggagcattgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctgggatcccagccaggtttagtgccagtgggtctgggacagacttcaccctcaacatccatcctgtggaggaggaggatactgcaacctattactgtcagcaaagtaatgaggatccgtacacgttcggaggggggaccaagctggagctgaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 50 - DNA, 708 bp, Artificial
  VH(anti-CD16)-CH1
atggagttgcctgttaggctgttggtgctgatgttctggattcctgcttccagcagccaggttactctgaaagagtctggccctgggatattgcagccctcccagaccctcagtctgacttgttctttctctgggttttcactgaggacttctggtatgggtgtaggctggattcgtcagccttcagggaagggtctagagtggctggcacacatttggtgggatgatgacaagcgctataatccagccctgaagagccgactgacaatctccaaggatacctccagcaaccaggtattcctcaaaatcgccagtgtggacactgcagatactgccacatactactgtgctcaaataaaccccgcctggtttgcttactggggccaagggactctggtcactgtctctgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtttag

SEQ ID NO: 51 - protein, 674 aa, Artificial
  VH(anti-CD20)-CH1-H-CH2-CH3-L-VL(anti-CD16)-CL (L = G4S)
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 216 aa, Artificial
  VH(anti-CD16)-CH1
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 53 - DNA, 642 bp, Artificial
  anti-CD47 Fab light chain
gatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 54 - protein, 215 aa, Artificial
  anti-CD47 Fab light chain
DIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 708 bp, Artificial; identical to SEQ ID NO: 50
  anti-CD47 Fab heavy chain

SEQ ID NO: 56 - protein, 221 aa, Artificial
  anti-CD47 Fab heavy chain
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 57 - DNA, 2121 bp, Artificial
  VH(anti-CD20)-CH1-H-CH2-CH3-L-VL(anti-CD47)-CL (L = (G4S)4)
atggagttgcctgttaggctgttggtgctgatgttctggattcctgctagctccagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggaggcagcggaggcggaggctccggcggaggaggctccggagatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 58 - DNA, 722 bp, Artificial
  VH(anti-CD47)-CH1-H
atggagttgcctgttaggctgttggtgctgatgttctggattcctgcttccagcagcgaggtgcagctggtggagtctgggggagacttagtgaagcctggagggtccctgaaactctcctgtgcagcctctggattcactttcagtggctatggcatgtcttgggttcgccagactccagacaagaggctggagtgggtcgcaaccattactagtggtggtacttacacctactatccagacagtgtgaaggggcgattcaccatctccagagacaatgccaagaacaccctgtacctgcaaatagacagtctgaagtctgaggatacagccatatatttctgtgcaagatccctcgcgggaaatgctatggactactggggtcaagggaccagcgtcaccgtctcctcagctagcaccaagggcccatcggtcttccccctggcaccctcctccaagagcacctctgggggcacacggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttag

SEQ ID NO: 59 - protein, 687 aa, Artificial
  VH(anti-CD20)-CH1-H-CH2-CH3-L-VL(anti-CD47)-CL (L = (G4S)4)
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGDIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 221 aa, Artificial; identical to SEQ ID NO: 56
  VH(anti-CD47)-CH1-H

SEQ ID NO: 61 - DNA, 642 bp, Artificial
  anti-CD52 Fab light chain
gacatccagatgacccagagcccaagcagcctgagcgccagcgtgggtgacagagtgaccatcacctgtaaagcaagtcagaatattgacaaatacttaaactggtaccagcagaagccaggtaaggctccaaagctgctgatctacaatacaaacaatttgcaaacgggtgtgccaagcagattcagcggtagcggtagcggtaccgacttcaccttcaccatcagcagcctccagccagaggacatcgccacctactactgcttgcagcatataagtaggccgcgcacgttcggccaagggaccaaggtggaaatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 62 - protein, 213 aa, Artificial
  anti-CD52 Fab light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 657 bp, Artificial
  anti-CD52 Fab heavy chain
caggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 64 - protein, 219 aa, Artificial
  anti-CD52 Fab heavy chain
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 65 - DNA, 2073 bp, Artificial
  VH(anti-CD20)-CH1-H-CH2-CH3-L-VL(anti-CD52)-CL (L = G4S)
atggagttgcctgttaggctgttggtgctgatgttctggattcctgctagctccagccaggtacaactgcaacagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgctgacaaatcctccagcacagcctacatgcagctcagcagcctgacatctgaggactctgcggtctattactgtgcaagatcgacttactacggcggtgactggtacttcaatgtctggggcgcagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgcaggtggaggtgggagcgacatccagatgacccagagcccaagcagcctgagcgccagcgtgggtgacagagtgaccatcacctgtaaagcaagtcagaatattgacaaatacttaaactggtaccagcagaagccaggtaaggctccaaagctgctgatctacaatacaaacaatttgcaaacgggtgtgccaagcagattcagcggtagcggtagcggtaccgacttcaccttcaccatcagcagcctccagccagaggacatcgccacctactactgcttgcagcatataagtaggccgcgcacgttcggccaagggaccaaggtggaaatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 66 - DNA, 732 bp, Artificial; identical to SEQ ID NO: 32
  VH(anti-CD52)-CH1-H

SEQ ID NO: 67 - protein, 670 aa, Artificial
  VH(anti-CD20)-CH1-H-CH2-CH3-L-VL(anti-CD52)-CL (L = G4S)
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 224 aa, Artificial
  VH(anti-CD52)-CH1-H
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - DNA, 2070 bp, Artificial
  VH(anti-CD52)-CH1-H-CH2-CH3-L-VL(anti-CD20)-CL (L = G4S)
atggagttgcctgttaggctgttggtgctgatgttctggattcctgctagctccagccaggtccaactgcaggagagcggtccaggtcttgtgagacctagccagaccctgagcctgacctgcaccgtgtctggcttcaccttcaccgatttctacatgaactgggtgagacagccacctggacgaggtcttgagtggattggatttattagagacaaagctaaaggttacacaacagagtacaatccatctgtgaaggggagagtgacaatgctggtagacaccagcaagaaccagttcagcctgagactcagcagcgtgacagccgccgacaccgcggtctattattgtgcaagagagggccacactgctgctccttttgattactggggtcaaggcagcctcgtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgcaggtggaggtgggagccaaattgttctctcccagtctccagcaatcctgtctgcatctccaggggagaaggtcacaatgacttgcagggccagctcaagtgtaagttacatccactggttccagcagaagccaggttcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 70 - DNA, 696 bp, Artificial
  VL(anti-CD52)-CL
atggagttgcctgttaggctgttggtgctgatgttctggattcctgcttccttaagcgacatccagatgacccagagcccaagcagcctgagcgccagcgtgggtgacagagtgaccatcacctgtaaagcaagtcagaatattgacaaatacttaaactggtaccagcagaagccaggtaaggctccaaagctgctgatctacaatacaaacaatttgcaaacgggtgtgccaagcagattcagcggtagcggtagcggtaccgacttcaccttcaccatcagcagcctccagccagaggacatcgccacctactactgcttgcagcatataagtaggccgcgcacgttcggccaagggaccaaggtggaaatcaaaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - protein, 669 aa, Artificial
  VH(anti-CD52)-CH1-H-CH2-CH3-L-VL(anti-CD20)-CL (L = G4S)
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 214 aa, Artificial
  VL(anti-CD52)-CL
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 1482 bp, Artificial
  H-CH2-CH3-L-VH(anti-CD47)-CH1-H (L = (G4S)4)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggcggcggaggaagcggaggaggtggcagcggtggcggtggctccggcggaggtggctccggagaggtgcagctggtggagtctgggggagacttagtgaagcctggagggtccctgaaactctcctgtgcagcctctggattcactttcagtggctatggcatgtcttgggttcgccagactccagacaagaggctggagtgggtcgcaaccattactagtggtggtacttacacctactatccagacagtgtgaaggggcgattcaccatctccagagacaatgccaagaacaccctgtacctgcaaatagacagtctgaagtctgaggatacagccatatatttctgtgcaagatccctcgcgggaaatgctatggactactggggtcaagggaccagcgtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttga

SEQ ID NO: 74 - DNA, 702 bp, Artificial
  VL(anti-CD47)-CL
atggagttgcctgttaggctgttggtgctgatgttctggattcctgcttccttaagcgatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 75 - protein, 474 aa, Artificial
  H-CH2-CH3-L-VH(anti-CD47)-CH1-H (L = (G4S)4)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGEVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - protein, 214 aa, Artificial
  VL(anti-CD47)-CL
DIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 1461 bp, Artificial
  H-CH2-CH3-L-VL(anti-CD47)-CL (L = (G4S)4)
atgaagcttcctgttaggctgttggtgctgatgttctggatccctgctagcttaagcgagcccaaatcttctgacaaaactcacacatgcccaccgtgcccagcacctgaactcctgggggggccctcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaacgatatccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcgccaccgcgaccccgggtgcaggcggcggaggaagcggaggaggtggcagcggtggcggtggctccggcggaggtggctccggagatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 78 - protein, 466 aa, Artificial
  H-CH2-CH3-L-VL(anti-CD47)-CL (L = (G4S)4)
EPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGDIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - DNA, 2115 bp, Artificial
  VH(anti-EGFR)-CH1-H-CH2-CH3-L-VL(anti-CD47)-CL (L = (G4S)4)
atggagttgcctgttaggctgttggtgctgatgttctggattcctgctagctccagccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggaggcagcggaggcggaggctccggcggaggaggctccggagatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 80 - protein, 685 aa, Artificial
  VH(anti-EGFR)-CH1-H-CH2-CH3-L-VL(anti-CD47)-CL (L = (G4S)4)
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGDIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 2058 bp, Artificial
  VH(anti-CD47)-CH1-H-CH2-CH3-L-VL(anti-EGFR)-CL (L = (G4S)4)
atggagttgcctgttaggctgttggtgctgatgttctggattcctgctagctccagcgaggtgcagctggtggagtctgggggagacttagtgaagcctggagggtccctgaaactctcctgtgcagcctctggattcactttcagtggctatggcatgtcttgggttcgccagactccagacaagaggctggagtgggtcgcaaccattactagtggtggtacttacacctactatccagacagtgtgaaggggcgattcaccatctccagagacaatgccaagaacaccctgtacctgcaaatagacagtctgaagtctgaggatacagccatatatttctgtgcaagatccctcgcgggaaatgctatggactactggggtcaagggaccagcgtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctccagtggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatccagcatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacacagaagagcctctccctgtccccgggtgcaggtggaggtgggagcgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctggaatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 82 - protein, 666 aa, Artificial
  VH(anti-CD47)-CH1-H-CH2-CH3-L-VL(anti-EGFR)-CL (L = (G4S)4)
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - DNA, 642 bp, Artificial; identical to SEQ ID NO: 53
  anti-HER2 Fab light chain

SEQ ID NO: 84 - protein, 215 aa, Artificial; identical to SEQ ID NO: 54
  anti-HER2 Fab light chain

SEQ ID NO: 85 - DNA, 660 bp, Artificial
  anti-HER2 Fab heavy chain
gaggtgcagctggtggagtctgggggagacttagtgaagcctggagggtccctgaaactctcctgtgcagcctctggattcactttcagtggctatggcatgtcttgggttcgccagactccagacaagaggctggagtgggtcgcaaccattactagtggtggtacttacacctactatccagacagtgtgaaggggcgattcaccatctccagagacaatgccaagaacaccctgtacctgcaaatagacagtctgaagtctgaggatacagccatatatttctgtgcaagatccctcgcgggaaatgctatggactactggggtcaagggaccagcgtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatct

SEQ ID NO: 86 - protein, 221 aa, Artificial; identical to SEQ ID NO: 56
  anti-HER2 Fab heavy chain

SEQ ID NO: 87 - DNA, 2118 bp, Artificial
  VH(anti-HER2)-CH1-H-CH2-CH3-L-VL(anti-CD47)-CL (L = (G4S)4)
atggagttgcctgttaggctgttggtgctgatgttctggattcctgctagctccagcgaggtgcaactggtggagagcggaggaggcctcgtgcaacccggaggatccctcagactgagctgtgccgccagcggcttcaatatcaaggatacctatatccactgggtgaggcaggcccccggaaaaggactggagtgggtggccaggatctatcccacaaacggctacaccaggtacgccgattccgtgaagggcagattcaccatcagcgccgatacctccaaaaacaccgcctatctccagatgaacagcctcagagccgaggacaccgccgtctattactgttccagatggggcggcgacggcttttacgctatggattactggggccagggaaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggaggcagcggaggcggaggctccggcggaggaggctccggagatattgtgatgactcagtctccagccaccctgtctgtgactccaggagatagagtctctctttcctgcagggccagccagactattagcgactacttacactggtatcaacaaaaatcacatgagtctccaaggcttctcatcaaatttgcttcccaatccatttctggaatcccctccaggttcagtggcagtggatcaggctcagatttcactctcagtatcaacagtgtggaacctgaagatgttggagtgtattactgtcaaaatggtcacggctttcctcggacgttcggtggagggaccaagctggaaataaaacgtggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 88 - DNA, 702 bp, Artificial
  VL(anti-HER2)-CL
atggagttgcctgttaggctgttggtgctgatgttctggattcctgcttccttaagcgacatccagatgacccagagccctagcagcctgagcgcgagcgtgggcgacagagtgacaatcacctgcagggccagccaggacgtgaataccgccgtggcctggtaccagcagaaacccggcaaggcccctaagctgctgatctactccgcctccttcctctacagcggcgtgcccagcaggtttagcggcagcaggagcggcacagatttcaccctgaccatcagcagcctgcagcccgaggacttcgccacctactactgccagcagcattacaccaccccccccaccttcggccagggaacaaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 89 - protein, 683 aa, Artificial
  VH(anti-HER2)-CH1-H-CH2-CH3-L-VL(anti-CD47)-CL (L = (G4S)4)
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGDIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, Artificial; identical to SEQ ID NO: 76
  VL(anti-HER2)-CL

SEQ ID NO: 91 - DNA, 2058 bp, Artificial
  VH(anti-CD47)-CH1-H-CH2-CH3-L-VL(anti-HER2)-CL (L = (G4S)4)
atggagttgcctgttaggctgttggtgctgatgttctggattcctgctagctccagcgaggtgcagctggtggagtctgggggagacttagtgaagcctggagggtccctgaaactctcctgtgcagcctctggattcactttcagtggctatggcatgtcttgggttcgccagactccagacaagaggctggagtgggtcgcaaccattactagtggtggtacttacacctactatccagacagtgtgaaggggcgattcaccatctccagagacaatgccaagaacaccctgtacctgcaaatagacagtctgaagtctgaggatacagccatatatttctgtgcaagatccctcgcgggaaatgctatggactactggggtcaagggaccagcgtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctccagtggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatccagcatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacacagaagagcctctccctgtccccgggtgcaggtggaggtgggagcgacatccagatgacccagagccctagcagcctgagcgcgagcgtgggcgacagagtgacaatcacctgcagggccagccaggacgtgaataccgccgtggcctggtaccagcagaaacccggcaaggcccctaagctgctgatctactccgcctccttcctctacagcggcgtgcccagcaggtttagcggcagcaggagcggcacagatttcaccctgaccatcagcagcctgcagcccgaggacttcgccacctactactgccagcagcattacaccaccccccccaccttcggccagggaacaaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 92 - DNA, 729 bp, Artificial
  VH(anti-HER2)-CH1-H
atggagttgcctgttaggctgttggtgctgatgttctggattcctgcttccagcagcgaggtgcaactggtggagagcggaggaggcctcgtgcaacccggaggatccctcagactgagctgtgccgccagcggcttcaatatcaaggatacctatatccactgggtgaggcaggcccccggaaaaggactggagtgggtggccaggatctatcccacaaacggctacaccaggtacgccgattccgtgaagggcagattcaccatcagcgccgatacctccaaaaacaccgcctatctccagatgaacagcctcagagccgaggacaccgccgtctattactgttccagatggggcggcgacggcttttacgctatggattactggggccagggaaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcgccgtgatcagcagcggcggcagctccatcaactacaagaaagttgagcccaaatcttgttaa

SEQ ID NO: 93 - protein, 666 aa, Artificial
  VH(anti-CD47)-CH1-H-CH2-CH3-L-VL(anti-HER2)-CL (L = (G4S)4)
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSDIVMTQSPATLSVTPGDRVSLSCRASQTISDYLHWYQQKSHESPRLLIKFASQSISGIPSRFSGSGSGSDFTLSINSVEPEDVGVYYCQNGHGFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 221 aa, Artificial
  VH(anti-HER2)-CH1-H
EVQLVESGGDLVKPGGSLKLSCAASGFTFSGYGMSWVRQTPDKRLEWVATITSGGTYTYYPDSVKGRFTISRDNAKNTLYLQIDSLKSEDTAIYFCARSLAGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2016/0009824 A1
(43) Pub. Date: Jan. 14, 2016

(54) TETRAVALENT BISPECIFIC ANTIBODIES

(71) Applicant: MERCK PATENT GMBH, Darmstadt (DE)

(72) Inventors: Kin-Ming LO, Lexington, MA (US); Nora A.E. ZIZLSPERGER, Newton, MA (US)

(21) Appl. No.: 14/777,152

(22) PCT Filed: Mar. 14, 2014
(86) PCT No.: PCT/US2014/028731

§ 371 Date: Sep. 15, 2015

Related U.S. Application Data
(60) Provisional application No. 61/793,153, filed on Mar. 15, 2013.

(51) Int. Cl. C07K 16/46; C07K 16/32; C07K 16/28

(52) U.S. Cl. CPC C07K 16/468; C07K 16/2863; C07K 16/2887; C07K 16/2803; C07K 16/2893; C07K 16/283; C07K 16/32; C07K 2317/55; C07K 2317/524; C07K 2317/31; C07K 2317/35; C07K 2317/522; C07K 2317/56; C07K 2317/76; C07K 2317/526; C07K 2317/64; C07K 2317/66; C07K 2319/33

(57) ABSTRACT

The present invention relates to tetravalent bispecific antibodies (TetBiAbs), methods of making and methods of using the same for diagnostics and for the treatment of cancer or immune disorders. TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

### REFERENCE TO RELATED APPLICATIONS

This application claims the benefit of and priority to U.S. Provisional Patent Application No. 61/793,153, filed Mar. 15, 2013, the complete disclosure of which is incorporated by reference herein.

### FIELD OF THE INVENTION

The present invention relates to tetravalent bispecific antibodies (TetBiAbs), methods of making and methods of using the same for the treatment of cancer or immune disorders and for diagnostics.

### BACKGROUND

Recent technological advances in antibody engineering have focused on using bispecific approaches to (1) engage effector cells for redirected lysis of tumor cells, (2) increase binding avidity and specificity of the targeting, or to (3) combine two drug candidates in one for regulatory and commercial reasons. In the first approach, the bispecific antibody acts as a bridge between the disease-causing cell and an effector cell through engagement of CD3 (Baeuerle et al, Cancer Res. 69:4941, 2009), CD16 (Weiner et al, Cancer Immunol. Immunother. 45:190, 1997), or CD64 (Graziano et al, Cancer Immunol. Immunother. 45:124, 1997) for redirected lysis. In the second approach, the selectivity for a target or a target cell can be significantly increased by combining two antibodies with mediocre binding affinities into a biparatopic (binding to two distinct epitopes on the same target antigen) or a bispecific (binding to two different antigens on the same target cell) antibody, respectively. The third approach is exemplified by simultaneous binding of two soluble cytokines (Mabry et al, Protein Eng. Des. & Sel. 23:115, 2010; Wu et al, Nature Biotech. 25:1290, 2007), which exploits the potential synergism of dual targeting in the appropriate disease setting. In addition to providing exquisite binding specificity through the variable regions, IgG also has effector functions and a very long serum half-life, and therefore, is often the preferred backbone for designing bispecific antibodies.

Current bispecific antibody technologies mostly rely on the scFv (single-chain fragment of the variable regions) format (Coloma and Morrison, Nature Biotechnol. 15:159, 1997; Lu et al, J. Biol. Chem. 280:19665, 2005) in which each VH (variable region of the heavy chain) is covalently linked to its cognate VL (variable region of the light chain), because in a Fab format there is yet no existing technology that can direct the specific pairing of a free light chain to only its cognate heavy chain and therefore the free light chains of different antigen specificity pair randomly with the heavy chains. However, expression of single-chain antibodies is often technically challenging, due to possible loss of binding affinity, protein aggregation, poor stability, and low production level (Demarest et al, Curr. Opin. Drug Discov. Devel. 11:675, 2008; Michaelson et al, mAbs 1:2, 128-141, 2009). This is especially true if the starting antibody is from a hybridoma (as opposed to a single-chain antibody from a phage display library) that has to be reformatted into a single-chain antibody. On the other hand, scFv's isolated from phages often are expressed poorly in mammalian cells.

Several innovative technologies have enabled the almost exclusive assembly of the Fc heterodimer to provide the backbone for designing bispecificity, e.g. knob-in-hole (Ridgway et al, Protein Eng. 9:617, 1996), electrostatic steering (Gunasekaran et al, J. Biol. Chem. 285:19637, 2010) and strand-exchange engineering domain (SEED) (Davis, Protein Eng. Des. & Sel. 23:195, 2010). However, there is yet no existing technology that can direct the specific pairing of a free light chain to only its cognate heavy chain that would allow for the engineering of a bispecific antibody relying on a native heavy chain-light chain Fab format. Due to the aforementioned problems with the scFv format, some technologies screen for a common light chain for the two different Fab's (Merchant et al, Nature Biotechnol. 16:677, 1998), or use single variable domains to avoid the use of the light chain altogether (Shen et al, J. Immunol. Methods 318:65, 2007).

In the Dual Variable Domains (DVD)-Ig approach, the VL and VH of the second antibody are fused via flexible linkers to the N-termini of the light and heavy chains, respectively, of the first antibody, creating two variable domains (VD) in tandem, called the outer VD and the inner VD (Wu et al, ibid). Due to the steric hindrance caused by the proximity of the outer VD to the ligand-binding site of the inner VD, extensive optimization involving VD selection from a number of available monoclonal antibodies, orientation of VDs, and linker designs, most of which have to be empirically determined, is necessary to retain the binding affinity of the inner VD (DiGiammarino et al, Methods Mol. Biol. 899:145, 2012).

Another method takes advantage of the preferential species-restricted heavy and light chain pairing in rat/mouse quadromas (Lindhofer et al, J. Immunol. 155:219, 1995). However, the bispecific antibody generated is a rat/mouse antibody, which obviously has immunogenicity issues as a therapeutic.

The Crossmab approach, based on the knob-into-hole heterodimerized heavy chains, in addition uses immunoglobulin domain crossover as a generic approach for the production of bispecific IgG antibodies (Schaefer et al, Proc. Natl. Acad. Sci. USA, 108:11187, 2011). Nevertheless, the correct pairings of the H chain heterodimer and the cognate Fv's are not exclusive, and the unwanted side products have to be removed during purification.

An extension of the Crossmab approach was used to generate a tetravalent bispecific antibody by tagging an extra set of Fab and Crossmab Fab fragments to the C-termini of Crossmab (Regula et al, US Patent Application No: 2010/0322,934), and the challenges of obtaining exclusively correct pairings of the H chain heterodimer and the cognate Fv's remain.

A further approach to bispecificity is to use a single binding site to target two different antigens was demonstrated by a “two-in-one” antibody. One such “two-in-one” antibody is a variant of the antibody Herceptin, which interacts with both Her2 and VEGF (Bostrom et al, Science 323:1610, 2009). This approach is attractive for clinical applications because it provides a bispecific antibody that has an identical format as a normal IgG. However, screening for such a variant is very labor intensive and there is no guarantee that a single binding site which can bind both antigens of interest can be obtained.

A stable multivalent antibody with only monospecificity based on a single set of Fab fragments was described in US published patent application US2011/0076722. Another technology uses Dock-and-Lock domains to link preformed Fab fragments of a different specificity to an antibody to form a hexavalent bispecific antibody (Rossi et al, Cancer Res. 68:8384, 2008).

Since the vast majority of antibodies (i.e. those generated from hybridomas, Fab libraries and B-cell cloning, regardless of whether the origin is from normal mice, rats, and rabbits, or transgenic (humanized) mice or rats, or patients) have a free light chain paired with its cognate heavy chain, a Fab-based technology for bispecific antibodies that circumvents the problem of random light chain pairing is urgently needed. Such a technology would facilitate straightforward and efficient production of a bispecific antibody from two existing antibodies, which can be used first as a versatile tool molecule to probe the potential synergism of dual targeting, and secondly as a therapeutic to exploit the dual targeting in the context of a complete antibody in the disease setting to be treated.

### SUMMARY OF THE INVENTION

The present invention features tetravalent bispecific antibodies (TetBiAb). In a general embodiment of the invention, the antibody contains an antibody Fc region linked at its C-terminus by means of Fab light chains to a Fab. In one embodiment of a TetBiAb, an antibody is covalently linked at its C-terminus by means of Fab light chains to a second pair of Fabs with a second binding specificity, wherein the linked Fab light chain is paired with a free cognate Fab heavy chain. Conversely, the Fab heavy chain at the N-terminus of the antibody pairs as usual with its cognate free light chain. The resulting antibody is bivalent for each of its binding specificities. The arrangement of the polypeptide chains in a TetBiAb is schematically depicted in FIG. 1B.

In an alternate embodiment of a TetBiAb, an antibody Fc region is linked at its N-terminus by means of Fab light chains to a Fab of a first specificity, wherein the linked Fab light chain is paired with a free cognate Fab heavy chain, and additionally, the antibody Fc region is linked at its C-terminus by means of Fab heavy chains to a Fab of a second specificity. The linked Fab heavy chain at the C-terminus of the antibody pairs as usual with its cognate free light chain. Again, the resulting antibody is bivalent for each of its binding specificities. The arrangement of the polypeptide chains in this alternate TetBiAb is schematically depicted in FIG. 1D.

Thus, in one embodiment of the invention, a TetBiAb comprises (i) a first polypeptide, comprising an antibody heavy chain of a first antibody, wherein the heavy chain contains a variable domain and constant domains of the first antibody (VH(1)-CH1-hinge-CH2-CH3), where the heavy chain is linked at its C-terminus, either directly or indirectly, by a peptide bond to the N-terminus of an antibody light chain of a second antibody, wherein the light chain contains a variable and a constant domain of the second antibody (VL(2)-CL); (ii) a second polypeptide comprising the antibody light chain of the first antibody, wherein the light chain of the first antibody contains a variable and a constant domain (VL(1)-CL); and (iii) a third polypeptide comprising the Fab heavy chain of the second antibody and lacking CH2 and CH3 constant domains (VH(2)-CH1). It is understood that the first and second antibodies have different binding specificities, i.e., the antibodies specifically bind to distinct epitopes. These polypeptides assemble into a complete tetravalent bispecific antibody In a further embodiment of the invention, the first polypeptide of the TetBiAb (VH(1)-CH1-hinge-CH2-CH3-(L)-VL(2)-CL) further comprises a linker operably linking the C-terminus of the heavy chain constant domains to the N-terminus of the light chain variable domain. In ine embodiment, the linker has the amino acid sequence (GGGGS)ₙ (SEQ ID NO:6), wherein n is an integer between 1 and 10. In yet a further embodiment the linker is a (GGGGS)ₙ where n is 4.

In a further embodiment of the invention, the heavy chain constant domains of said first polypeptide of the TetBiAb are IgG constant domains.

In a further embodiment of the invention, said first polypeptide of the TetBiAb lacks a CH2 domain.

In a further embodiment of the invention, the third polypeptide, (VH(2)-CH1), includes an upper hinge region at its C-terminus, having the sequence EPKSC (SEQ ID NO:10).

In another aspect of the invention, DNA molecules are provided encoding the polypeptide chains forming the TetBiAb. In one embodiment, a DNA molecule comprising a first DNA sequence is provided, wherein the DNA sequence encodes a heavy chain of the first antibody (VH(1)-CH1-hinge-CH2-CH3) genetically fused via an optional linker to a light chain of a second antibody (VL(2)-CL), to give a sequence encoding VH(1)-CH1-hinge-CH2-CH3-(optional linker)-VL(2)-CL. In a further embodiment, a second DNA sequence is additionally provided to the first DNA sequence, wherein the second sequence encodes a light chain of the first antibody (VL(1)-CL). In a further embodiment, a third DNA sequence is additionally provided, wherein the third sequence encodes a Fab heavy chain of the second antibody (VH(2)-CH1), optionally linked to an additional sequence encoding a hinge region having the amino acid sequence EPKSC (SEQ ID NO:10). In a further embodiment, at least one of the first, second or third DNA sequences are contained on a separate DNA molecule.

In another embodiment of the invention, a DNA molecule containing a first, second and third gene construct is provided, wherein the first construct encodes the heavy chain of the first antibody (VH(1)-CH1-hinge-CH2-CH3) genetically fused via an optional linker to the light chain of a second antibody (VL(2)-CL) to give a sequence encoding VH(1)-CH1-hinge-CH2-CH3-optional linker-VL(2)-CL; the second construct encodes the light chain of the first antibody (VL(1)-CL); and the third construct encodes the Fab heavy chain of the second antibody (VH(2)-CH1), optionally linked to an additional sequence encoding a hinge region (amino acid sequence EPKSC, SEQ ID NO:10; see FIG. 1A).

The invention further provides for host cells carrying the DNA molecules of the invention.

The invention further provides for methods of producing the TetBiAbs of the invention.

In another aspect of the invention, methods to select appropriate target binding specificities for the TetBiAbs of the invention are provided.

Also provided are specific TetBiAbs. In one embodiment, the TetBiAb targets CD20 and CD16.

In another embodiment the TetBiAb targets EGFR and CD16. In a further embodiment the TetBiAb targets CD20 and CD47. In yet a further embodiment the TetBiAb targets CD20 and CD52. In yet a further embodiment, the TetBiAb targets EpCam and CD47.

One aspect of the invention provides methods of treating an individual having cancer or an immune related condition, with a TetBiAb of the invention, comprising administering to the individual a therapeutically effective amount of the TetBiAb, for example, TetBiAbs of the embodiments listed above, to treat the condition.

### BRIEF DESCRIPTION OF THE DRAWINGS

FIG. 1 schematically illustrates tetravalent bispecific antibodies (TetBiAbs). In FIG. 1A, DNA constructs for the expression of TetBiAbs are shown. DNA construct 1 (top) encodes the heavy chain variable domain of first antibody (VH(1)) followed by heavy chain constant domains (CH1, hinge (H)-CH2-CH3) genetically fused via an optional linker (L) to light chain variable domain of second antibody (VL(2)) followed by light chain constant domain (CL). DNA construct 2 (middle) encodes the light chain variable domain of first antibody (VL(1)) followed by light chain constant domain (CL). DNA construct 3 (bottom) encodes the heavy chain variable domain of the second antibody (VH(2)) followed by heavy chain constant domain 1 (CH1), and optionally an upper hinge region (H*). In FIG. 1B, a schematic drawing of a TetBiAb shows the hexameric structure comprising the three polypeptide components encoded by the DNA construct shown in FIG. 1A. Interchain disulfide bonds are depicted as short bars between two polypeptide chains. In FIG. 1C, alternate DNA constructs for the expression of TetBiAbs are shown. DNA construct 1 (top) encodes the light chain variable domain of a first antibody (VL(1)) followed by light chain constant domain (CL) followed by heavy chain constant domains (hinge (H)-CH2-CH3) genetically fused via an optional linker (L) to heavy chain variable domain of second a antibody (VH(2)) followed by heavy chain constant domain 1 (CH1), and optionally an upper hinge region (H*). DNA construct 2 (middle) encodes the light chain variable domain of the second antibody (VL(2)) followed by light chain constant domain (CL). DNA construct 3 (bottom) encodes the heavy chain variable domain of the first antibody (VH(1)) followed by constant domain 1 (CH1), and optionally an upper hinge region (H*). In FIG. 1D, a schematic drawing of a TetBiAb shows the hexameric structure comprising the three polypeptide components encoded by the DNA constructs shown in FIG. 1C. Interchain disulfide bonds are depicted as short bars between two polypeptide chains.

FIG. 2 shows by a competition binding assay with EGF the binding of anti-EGFR (filled circles, solid line), Fc-G4S-anti-EGFR(VHCH1) (open squares, dotted line), and Fc-G4S-anti-EGFR(LC) (filled squares, dashed lines) to human A431 epidermoid carcinoma cells expressing EGFR (Example 1).

FIG. 3 shows by SPR analysis the binding of EGFR at various concentrations to immobilized Fc-G4S-anti-EGFR(VHCH1), Fc-G4S-anti-EGFR(LC), and Fc-(G4S)₄-anti-EGFR(LC).

FIG. 4 shows the binding of anti-CD20 (filled circles, solid line), Fc-G4S-anti-CD20(VHCH1) (open triangles, dotted line), Fc-G4S₄-anti-CD20(VHCH1) (open squares, short dashed line), Fc-G4S-anti-CD20(LC) (filled triangles, solid line), and Fc-(G4S)₄-anti-CD20(LC) (filled squares, long dashed lines) to CD20 expressed on Daudi cells (Example 2).

FIG. 5 shows the analysis of the expression of the three polypeptides of anti-CD16/anti-EGFR (lane 2) and anti-EGFR/anti-CD16 (lane 3) by SDS-PAGE (FIG. 5A), and assembly of the full hexameric molecule of anti-CD16/anti-EGFR (upper panel) and anti-EGFR/anti-CD16 (lower panel) by size exclusion chromatography (SEC) (FIG. 5B; Example 3)).

FIG. 6 shows by a competition binding assay with EGF the binding of anti-EGFR (filled circles, solid line), anti-EGFR/anti-CD16 (open circle, dotted line), and anti-CD16/anti-EGFR (open squares, dashed lines) to human A431 epidermoid carcinoma cells expressing EGFR (Example 3).

FIG. 7 shows the antibody-dependent cell-mediated cytotoxicity (ADCC) activity of anti-EGFR (filled circles, solid line), anti-EGFR/anti-CD16 (open circle, dotted line), and anti-CD16/anti-EGFR (open squares, dashed lines) on human A431 epidermoid carcinoma cells, using resting human peripheral blood mononuclear cells (PBMCs) as effectors (effector-to-target cells ratio 100:1)(Example 4).

FIG. 8 shows the analysis of the expression of the three polypeptides of anti-CD20/anti-CD16 by SDS-PAGE (FIG. 8A) and assembly of the full hexameric molecule by size exclusion chromatography (SEC) (FIG. 8B).

FIG. 9 shows the binding of anti-CD20/anti-CD16 (open circles, dotted line) and anti-CD20 (filled circles, solid line) to Daudi cells expressing CD20.

FIG. 10 shows the antibody-dependent cell-mediated cytotoxicity (ADCC) activity of anti-CD20/anti-CD16 (open circles, dotted line) and anti-CD20 (filled circles, solid line) on human Ramos Burkitt's lymphoma cells, using purified human natural killer (NK) cells as effectors (effector-to-target cells ratio 10:1). The two graphs represent results with effector cells from different donors.

FIG. 11 shows the analysis of the expression of the three polypeptides of anti-CD20/anti-CD47 by SDS-PAGE (FIG. 11A) and assembly of the full hexameric molecule by size exclusion chromatography (SEC) (FIG. 11B; Example 5).

FIG. 12 shows binding of anti-CD20/anti-CD47 (open circles, dotted line), anti-CD20 (filled circles, solid line), and anti-CD47 (filled squares, solid line) to cells expressing either CD20 (CD20-transfected NS0 cells; FIG. 12A), CD47 (U937 cells; FIG. 12B), or both (SU-DHL4 cells; FIG. 12C).

FIG. 13 shows the analysis of the expression of the three polypeptides of anti-CD20/anti-CD52 (lane 2) and anti-CD52/anti-CD20 (lane 3) by SDS-PAGE (FIG. 13A) and assembly of the full hexameric molecule of anti-CD20/anti-CD52 (upper panel) and anti-CD52/anti-CD20 (lower panel) by size exclusion chromatography (SEC) (FIG. 13B; Example 6)).

FIG. 14 shows binding of anti-CD20/anti-CD52 (open circles, dotted line), anti-CD52/anti-CD20 (open triangles, dashed line), anti-CD20 (filled circles, solid line), and anti-CD52 (filled triangles, solid line) to cells expressing either CD20 (Daudi cells; FIG. 14A) or CD52 (Kasumi-3 cells, FIG. 14B)

FIG. 15 shows by ELISA the binding of Fc-(G4S)₄-anti-CD47(VHCH1) (open triangles, dotted line), Fc-(G4S)₄-anti-CD47(LC) (filled triangles, dashed lines), and anti-CD47 to immobilized CD47 at various antibody concentrations (Example 7).

FIG. 16 shows the analysis of the expression of the three polypeptides of anti-EGFR/anti-CD47 (lane 2) and anti-CD47/anti-EGFR (lane 3) by SDS-PAGE (FIG. 16A) and assembly of the full hexameric molecule of anti-EGFR/anti-CD47 (upper panel) and anti-CD47/anti-EGFR (lower panel) by size exclusion chromatography (SEC) (FIG. 16B; Example 8).

FIG. 17 shows binding by ELISA of anti-EGFR/anti-CD47 (open circle, dotted line), anti-CD47/anti-EGFR (open square, dashed line), anti-EGFR (filled circle, solid line), and anti-CD47 (filled square, solid line) to immobilized CD47 (FIG. 17A) or to immobilized EGFR (FIG. 17.B). Anti-EGFR/anti-CD47 (open circle, dotted line), anti-EGFR (filled circle, solid line) and anti-CD47 (filled square, solid line) binding to A431 cells (which express EGFR at high levels and CD 47 at low levels) is shown in FIG. 17C.

FIG. 18 shows the analysis of the expression of the three polypeptides of anti-HER2/anti-CD47 (lane 3) and anti-CD47/anti-HER2 (lane 3) by SDS-PAGE (FIG. 18A) and assembly of the full hexameric molecule of anti-HER2/anti-CD47 (upper panel) and anti-CD47/anti-HER2 (lower panel) by size exclusion chromatography (SEC) (FIG. 18B; Example 9).

FIG. 19 shows binding of anti-HER2/anti-CD47 (open triangles, dotted line), anti-CD47/anti-HER2 (open squares, dashed line), anti-HER2 (filled triangles, solid line), and anti-CD47 (filled squares, solid line), either by ELISA to immobilized CD47 (FIG. 19A), or to SK-BR3 cells, which express HER2 but not CD47 (FIG. 19B).

### DETAILED DESCRIPTION OF EMBODIMENTS OF THE INVENTION

The present invention overcomes a fundamental problem in the cellular expression, assembly and purification of a bispecific antibody comprising two Fab fragments with different binding specificities: the two species of free light chains randomly pair with Fab heavy chains, resulting in the production of multiple aberrant antibody species. These aberrant antibodies may be difficult to purify away from the desired product and affect product yield. In the technology of the present invention, only one species of free light chain is present and the desired bispecific antibody product is readily obtained.

In a general embodiment of the invention, the antibody contains an antibody Fc region, wherein the Fc heavy chains are linked at their C-termini by means of a Fab light chain to a Fab.

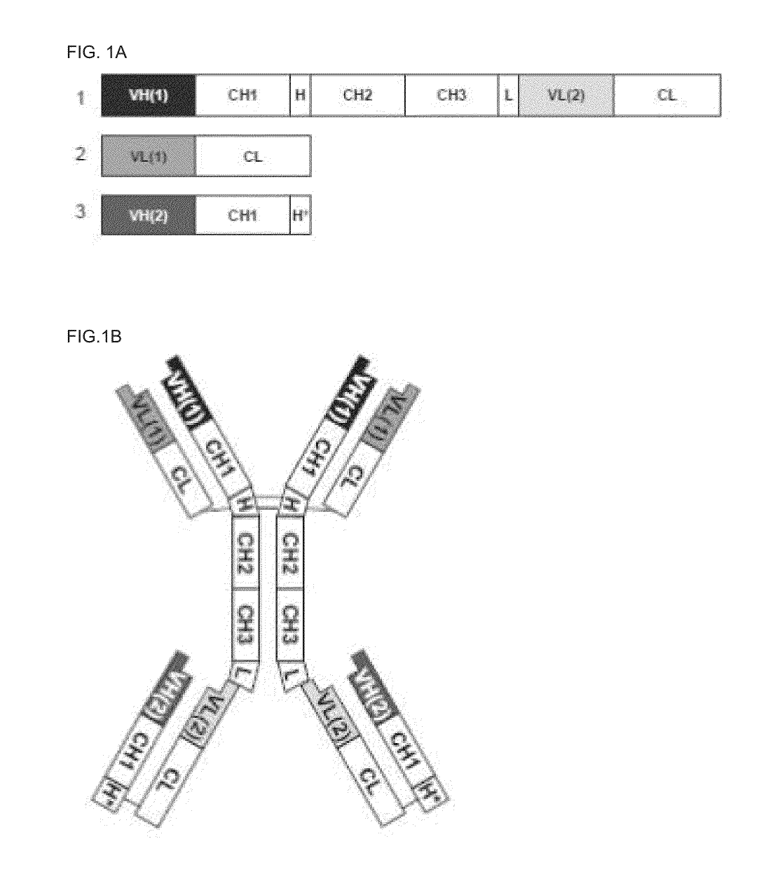

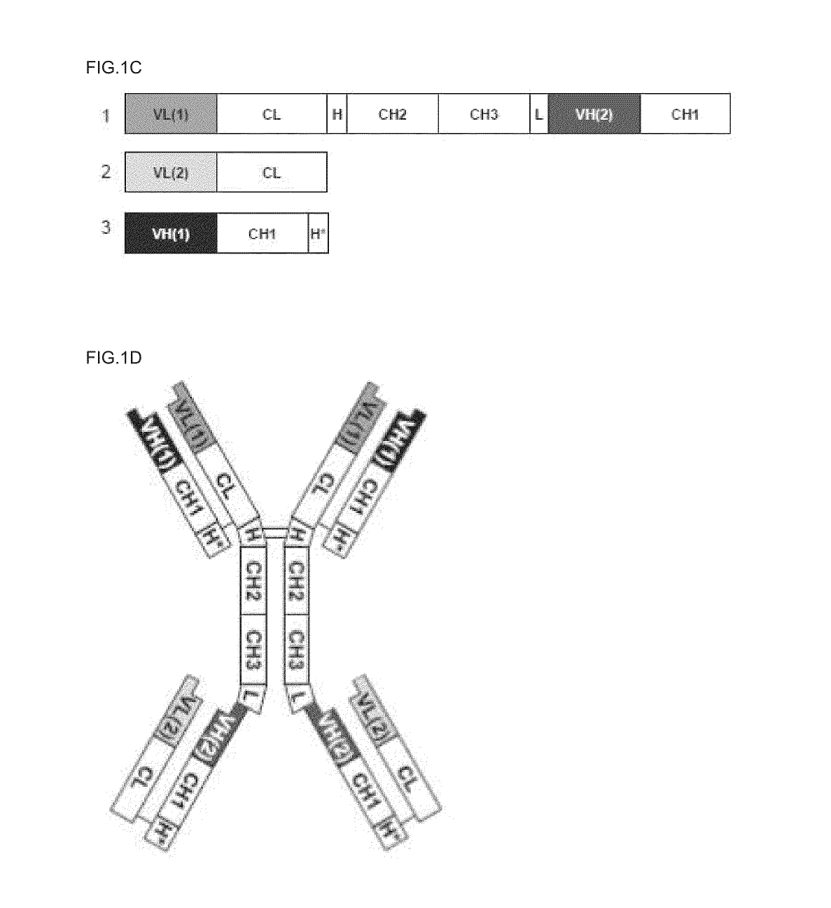

More specifically, the invention provides for tetravalent bispecific antibodies (TetBiAbs), in which a second Fab fragment with a second binding specificity is linked to the C-terminal ends of an antibody by means of the Fab light chains. These linked Fab light chains can then pair with free cognate Fab heavy chains. Conversely, the Fab heavy chain region normally residing at the N-terminus of the antibody can pair with its cognate free light chain. The resulting antibody is bivalent for each of its binding specificities. The arrangement of the polypeptide chains in a TetBiAb is schematically depicted in FIG. 1B.

A variation of the TetBiAb results in an inverted arrangement of the TetBiAb: the light chains are linked N-terminal the Fc polypeptide chains and the second set of Fabs with a second binding specificity are linked to the C-terminal ends of an Fc region by means of the Fab heavy chains. This arrangement of the polypeptide chains in a TetBiAb is schematically depicted in FIG. 1D.

The terms “Fab fragment” or simply “Fab” are used interchangeably, and are used herein to describe the antigen-binding portion of the antibody, essentially as obtained by papain digestion of an IgG antibody. The Fab fragment is heterodimeric, composed of two polypeptides, a light chain having a variable (VL) and constant (CL) domain, and a heavy chain having a variable (VH) and a first constant domain (CH1) and may also include the upper hinge region, particularly if the Fab is of a IgG1 subclass. The polypeptide chains are not linked to one another by a peptide bond but associate with one another by non-covalent interactions and by a disulfide bond if the upper hinge region of the heavy chain is present.

As used herein, the term “Fab heavy chain” denotes a polypeptide composed of a VH domain and a CH1 domain but does not contain a CH2 domain or a CH3 domain. The polypeptide may contain in addition the upper hinge region of the antibody hinge, particularly if the Fab is of a IgG1 subclass.

As used herein, the term “light chain” (LC) or “Fab light chain” denotes a polypeptide composed of a VL domain and a CL domain. Antibody light chains are classified as either kappa or lambda light chains or kappa.

As used herein, the term “free light chain” or “free Fab heavy chain” describes a polypeptide component of the antibody of the invention that is not linked to the Fc polypeptide chain by a peptide bond.

As used herein, the term “Fc region” describes the portion of the antibody which binds to Fc receptors and certain complement proteins, and essentially corresponds to the fragment traditionally obtained by papain digestion but including the upper hinge region. The Fc region is typically homodimeric, composed of two identical polypeptide chains derived from the antibody heavy chain, typically containing the hinge, a CH2 and a CH3 domain, but not a CH1 domain (“Fc heavy chain”; in a IgG1 polypeptide, the Fc heavy chain hinge begins at residue 216 as defined by the EU numbering system, corresponding to the amino acid glutamate). In certain embodiments the CH2 domain may be lacking. In other embodiments, the Fc region may contain mutations that affect, for example, effector function engagement or antibody half-life. The polypeptide chains associate with one another by non-covalent interactions in the CH3 domain and disulfide bonds in the hinge domain.

As used herein, the term “domain” describes a structurally or functionally defined element or constituent part of, for example, a protein or polypeptide chain. An example of a Fc heavy chain constant domain is a CH2 domain or a CH3 domain. An example of a Fab domain is a light chain variable domain (VL) or a Fab heavy chain constant domain (CH1).

As used herein, the terms “monovalent”, “bivalent”, “tetravalent” refer to the number (one, two or four, respectively) of antigen binding elements in a protein.

As used herein, a specific TetBiAb is designated as “anti-Target(1)/“anti-Target(2)”, wherein the order of the targets in the designation reflects the order of the Fab fragments relative to the Fc region. Anti-Target(1)/Anti-Target(2) has the order Fab(anti-Target(1))-Fc-Fab(anti-Target(2)).

In a general embodiment of the invention, a TetBiAb comprises (i) a first polypeptide, comprising an antibody heavy chain of a first antibody, wherein the heavy chain contains a variable domain and constant domains of the first antibody (VH(1)-CH1-hinge-CH2-CH3), where the heavy chain is linked at its C-terminus, either directly or indirectly, by a peptide bond, to the N-terminus of an antibody light chain of a second antibody, wherein the light chain contains a variable and constant domain of the second antibody (VL(2)-CL); (ii) a second polypeptide comprising the antibody light chain of the first antibody, wherein the light chain of the first antibody contains variable and constant domains (VL(1)-CL); and (iii) a third polypeptide comprising the Fab heavy chain of the second antibody and lacking the CH2 and CH3 constant domains (VH(2)-CH1). It is understood that the first and second antibodies have different binding specificities, i.e., the antibodies specifically bind to distinct epitopes. These polypeptides assemble into a complete tetravalent bispecific antibody.

In a further embodiment, the first polypeptide may contain a linker between the C-terminus of the heavy chain constant domain and the N-terminus of the light chain variable domain. In one embodiment the linker is G4S (amino acid sequence GGGGS, SEQ ID NO:6). The linker may contain multiple, concatenated G4S elements, (G4S)ₙ, where n is an integer between 2 and 10. In a further embodiment, n is an integer between 2 and 6. In yet a further embodiment n is 4.

In a further embodiment the free Fab heavy chain polypeptide, VH(2)-CH1 of the TetBiAb described above, further comprises at its C-terminus an Fc hinge region of the amino acid sequence EPKSC (SEQ ID NO:10; “upper hinge region”), which allows the heavy chain polypeptide to form a disulfide bond with its cognate light chain.

In another aspect of the invention, DNA constructs are provided encoding the three polypeptide chains forming the TetBiAb. The first construct encodes a heavy chain of the first antibody (VH(1)-CH1-hinge-CH2-CH3) genetically fused via an optional linker to a light chain of a second antibody (VL(2)-CL) to give the DNA sequence encoding VH(1)-CH1-hinge-CH2-CH3-optional linker-VL(2)-CL; the second construct encodes a light chain of the first antibody (VL(1)-CL); and the third construct encodes a Fab heavy chain of the second antibody (VH(2)-CH1), optionally with in addition the sequence encoding a hinge region (amino acid sequence EPKSC, SEQ ID NO:10; see FIG. 1A).

In another embodiment the DNA construct encodes a fusion polypeptide, comprising a light chain of the first antibody (VL(1)-CL1) genetically fused to the hinge-CH2-CH3 followed by an optional linker and a Fab heavy chain of a second antibody (VH(2)-CH1) to give the sequence VL(1)-CL1-hinge-CH2-CH3-optional linker-VH(2)-CH1 (FIG. 1C).

In a further aspect of the invention, methods to produce the TetBiAbs of the invention are provided. Upon coexpression of the three DNA constructs in appropriate expression vectors containing signal peptides for secretion in a host cell, the desired TetBiAb with the two different binding specificities (FIG. 1B) is formed and secreted into the culture media, and is purified by standard antibody purification procedures such as protein A chromatography. An example of a suitable host cell for transient expression is the human embryonic kidney cell 293E. An example of a suitable host cell for stable expression is the Chinese hamster ovary (CHO) cell.

It is evident to a person skilled in the art that the three expressed polypeptide chains are not linked to one another by peptide bonds. This invention takes advantage of the fact that there is only one free light chain (VL-CL), so that the random light chain pairing problem is overcome. Another advantage of the invention is the fact that the Fab fragment is very stable, compared to scFv, and an antibody with an extra Fab fragment fused to the C-terminus of its heavy chain is expected to be very stable and produced at a high level in general. Importantly, this invention is based on the expression of one single species of antibody heavy chain fusion polypeptide chain, which pairs specifically with one cognate free light chain polypeptide at one end and one cognate free Fab heavy chain polypeptide at the other end of the fusion polypeptide. Hence a heterodimeric Fc backbone is not needed to provide the means for assembling a bispecific antibody. Therefore, there is no mis-pairing of heavy chains.

It is an object of the invention to provide DNAs that are modular in nature, with respect to the variable regions of the first and second antibody, so that cDNAs encoding the VH and VL of the first and second antibody can be readily assembled without having to introduce, for example, stabilizing mutations and extensive optimization for expression of a bispecific antibody. Such a robust technology to facilitate the production of a bispecific antibody is highly advantageous in discovery of target combinations that may yield synergistic effect in certain disease settings.

Another object of the invention is to provide a stable antibody-based fusion protein suitable for development as a biotherapeutic, featuring Fab fragments to accomplish bispecific binding, and an Fc region, optionally altered, to achieve the desired effector function and half-life profile. Fc variants that affect effector functionand half-life are well understood in the art (see, for example WO 2000/042072). It is well appreciated in the art that Fab fragments are intrinsically more stable than single-chain Fv's (Rothlisberger et al, J. Mol. Biol. 347:773, 2005), they occur naturally as the binding arms of an antibody, and can be used as such without further engineering (Schoonjans et al, J. Immunol. 165:7050, 2000).

In one embodiment, the human IgG1 constant regions and the kappa constant regions are used for the construction of TetBiAbs. To date, all approved therapeutic antibodies are of the immunoglobulin G (IgG) isotype because IgGs are the predominant serum immunoglobulins and are readily manufacturable as biotherapeutics. Furthermore, IgG binds the Fcγ receptors (FcγR) on immune cells to elicit various effector functions and is the only isotype that binds the protective neonatal Fc receptor FcRn, which gives typical IgGs their long serum half-lives in humans. Within the IgG isotype, there are four subclasses, namely IgG1, IgG2, IgG3 and IgG4. The IgG subclass of the antibody, which determines its effector functions, is carefully chosen to suit its therapeutic applications. Accordingly, the IgG1 subclass is chosen when effector functions are desirable, IgG2 is chosen for its lack of FcγR binding to minimize antibody-dependent cellular cytotoxicity (ADCC), and IgG4 is chosen for its low ADCC activity and complete lack of complement-dependent cytotoxicity (CDC). Constant regions of the other immunoglobulin isotypes, such as IgA, IgD, IgE and IgM can also be used for constructing the TetBiAbs. In addition to the heavy chain constant region sequences from the natural isotypes and IgG subclasses, recombinant hybrid isotypes can also be used in this invention (e.g. Gillies, S. D., and Lo, K.-M. Expression technology for proteins containing a hybrid isotype antibody moiety. U.S. Pat. No. 7,148,321). Furthermore, the CH1 used for the C-terminal Fab can be of a different isotype from the CH1 used in the N-terminal Fab. Moreover, if a CH1 of IgG1 is used for the C-terminal Fab, the CH1 may be extended at its C-terminus by an additional five residues EPKSC (SEQ ID NO:10) from the IgG1 upper hinge region, in order to provide the cysteine residue that normally forms a disulfide bond with the light chain (Röthlisberger et al, J Mol Biol. 347:773, 2005). For the light chain constant region, the kappa chain constant region or the lambda chain constant region are used for either the N-terminal Fab or C-terminal Fab, or both

Another object of the invention is to provide TetBiAbs as diagnostic agents with more specific detection, extended dissociation half-times, and improved sensitivity in assays such as Luminex and other multiplex assays, and increase the specific binding of target cells in fluorescence-activated cell sorting (FACS) analysis.

In another aspect, the invention provides methods of producing a TetBiAb for therapeutic application. The method comprises the steps of (a) providing a mammalian cell containing transfected DNA molecules encoding such a tetravalent bispecific antibody; (b) culturing the mammalian cell to produce the tetravalent bispecific antibody; (c) purifying the tetravalent bispecific antibody using conventional techniques well known in the art; and (4) formulating the TetBiAb for therapeutic application. Just like natural antibodies, the TetBiAb retains bivalent binding per target, but in addition, avidity of binding to the disease-causing cell is increased through binding to two disease-related targets on the same cell, resulting in more specific targeting and less side effects. Furthermore, such increased avidity can provide extensive multivalent crosslinking of receptors that often enhance biological activities such as growth arrest, apoptosis, and receptor internalization and degradation. Overall, the multivalent binding and high avidity of a TetBiAb are characteristics that in therapeutic applications have potential for leading to decreased therapeutic dosages and increased efficacy.

Specific non-limiting embodiments for tetravalent bispecific antibodies include anti-EGFR/anti-CD16 (Example 3), anti-CD20/anti-CD16 (Example 4), anti-CD20/anti-CD47 (Example 5), anti-CD20/anti-CD52 (Example 6), anti-EGFR/anti-CD47 (Example 8) and anti-Her2/anti-CD47 (Example 9) in which the specificity of the first antibody is comprised on the N-terminal Fab and the specificity of the second antibody is comprised on the C-terminal Fab (see FIG. 1). The positions of the two antibodies can be reversed, for example, anti-EGFR/anti-CD16 instead of anti-CD16/anti-EGFR.

One skilled in the art can express both forms of the tetravalent bispecific antibody and then determine which is the preferred form based on expression level, binding affinities of the N-terminal and C-terminal Fabs, and other biological activity assays. In one method, to simplify the construction of the DNA and the analysis of the fusion protein, one skilled in the art expresses the Fab-Fc (a normal antibody) and Fc-Fab for comparison, and determines which antibody Fab domain should be expressed as C-terminal Fabs.

One skilled in the art may also consider the nature of the target antigen in guiding the choice of which Fab to use as the C-terminally linked Fab. As a general rule, accessibility to the target antigen is more constrained at the binding site of the C-terminal Fab, and therefore soluble factors or receptors with large exposed extracellular domains are likely to be more amenable to targeting by a C-terminal Fab. Conversely, target antigens on multi-spanning membrane proteins with only small exposed extra-cellular loop regions or antigen surfaces close to the cell membrane may be less amenable to targeting by a C-terminal Fab.

Without being bound by theory, it is possible that the proximity of the Fc region to the binding site of the C-terminal Fab causes steric hindrance. For binding of a C-terminal Fab to a target, especially a cellular receptor, incorporation of a flexible linker may help to retain binding affinity by relieving steric hindrance. In one embodiment the flexible linker has the amino acid sequence GGGGS. One skilled in the art can readily test the optimal length of the flexible linker by incorporating multiple copies of the GGGGS sequence (SEQ ID NO:6). Generally, up to 10 copies are used, in one embodiment 4 copies are used.

Accordingly, in one embodiment the TetBiAb binds two distinct targets on two different cell types. Exemplary embodiments are an anti-EGFR/anti-CD16 or an anti-CD20/anti-CD16, in which the TetBiAb bridges between the EGFR or CD20 on a target tumor cell and the CD16 on a natural killer cell to direct the natural killer cell to the tumor. In another embodiment the tetravalent bispecific antibody binds two distinct targets on the same cell, such as exemplary embodiments anti-CD20/anti-CD47 or anti-CD20/anti-CD52. In yet another embodiment of the invention, the tetravalent bispecific antibody binds two different epitopes on the same molecular target (i.e. biparatopic). It is also apparent to the one skilled in the art that one or both of the targets of the TetBiAb can be soluble or expressed on a cell surface.

In one exemplary embodiment, the invention provides for an anti-CD20/anti-CD47 TetBiAb comprising an anti-CD20 heavy chain-anti-CD47 light chain fusion polypeptide, an anti-CD20 light chain, and an anti-CD47 Fab heavy chain, wherein:
(a) The VH and VL sequences of the anti-CD20 are identical to SEQ ID NO:24 and SEQ ID NO:22, respectively, and
(b) The VH and VL sequences of the anti-CD47 are identical to SEQ ID NO:56 and SEQ ID NO:54, respectively, and
(c) The constant regions are selected from the group consisting of human IgG1, IgG2, IgG3, IgG4, IgA, IgD, IgE, and IgM.

In a further embodiment, the invention provides for an anti-CD20/anti-CD47 tetravalent bispecific antibody comprising an anti-CD20 heavy chain-anti-CD47 light chain fusion polypeptide, an anti-CD20 light chain, and an anti-CD47 Fab heavy chain, wherein:
(a) The VH and VL sequences of the anti-CD20 have at least 85% sequence identity, at least 90%, at least 91%, at least 92%, at least 93%, at least 94%, at least 95%, at least 96%, at least 97%, or at least 98% to SEQ ID NO:24 and SEQ ID NO:22, respectively, and
(b) The VH and VL sequences of the anti-CD47 have at least 85% sequence identity, least 90%, at least 91%, at least 92%, at least 93%, at least 94%, at least 95%, at least 96%, at least 97%, or at least 98% to SEQ ID NO:56 and SEQ ID NO:54, respectively, and
(c) The constant regions are selected from the group consisting of human IgG1, IgG2, IgG3, IgG4, IgA, IgD, IgE, and IgM, including mutations to abrogate the effector functions of the Fc region.

In another exemplary embodiment, the invention provides for an anti-CD20/anti-CD47 tetravalent bispecific antibody comprising an anti-CD20 heavy chain-anti-CD47 light chain fusion polypeptide, an anti-CD20 light chain, and an anti-CD47 Fab heavy chain, wherein:
(a) The VH and VL sequences of the anti-CD20 comprise the complementarity-determining regions (CDRs) of SEQ ID NO:24 and SEQ ID NO:22, respectively, and consensus human framework regions (FRs); and
(b) The VH and VL sequences of the anti-CD47 comprise the complementarity-determining regions (CDRs) of SEQ ID NO:56 and SEQ ID NO:54, respectively, and consensus human framework regions (FRs); and
(c) The constant regions are selected from the group consisting of human IgG1, IgG2, IgG3, IgG4, IgA, IgD, IgE, and IgM, including mutations to abrogate the effector functions of the Fc region, or the group consisting of murine IgG1, IgG2a, IgG2b, IgG3, IgA, IgD, IgE, and IgM, including mutations to abrogate the effector functions of the Fc region.

According to another embodiment of the invention, TetBiAbs bind an antigen preferably expressed only on a disease-causing target cell, and is either not expressed or expressed at a low level in healthy tissues. Non-limiting examples of such target antigens include carcinoembryonic antigen, EGFR, EGFRvIII, IGF-1R, HER-2, HER-3, HER-4, MUC1, MUC-1C, EpCAM, PSMA, and gangliosides GD2 and GD3, many of which are tumor-specific antigens. In a TetBiAb, a Fab binding to any one of these tumor-specific antigens can be paired with a Fab that targets an antigen on an effector cell, such as antigens CD3 on a T cell, CD16 on an NK cell, or CD64 on a monocyte, to generate a TetBiAb that promotes lysis of the tumor cell. Such TetBiAbs can be used in the treatment of cancers characterized by the expression of these tumor antigens.

In an alternate embodiment of the invention, a TetBiAb binds an antigen that is expressed on the disease-causing cell and may also be expressed on a class of normal cells, such as is the case, for example, with antigens CD19 and CD20 expressed on normal and malignant B cells. In such a TetBiAb, a Fab binding to CD 19 or CD20 can be paired with a Fab that targets an effector cell, such as CD16 on an NK cell. For example, an anti-CD20/anti-CD16 TetBiAb may be used in the treatment of a hematological malignancy.

In yet another embodiment, a TetBiAb contains the Fabs of two antibodies, each antibody having otherwise mediocre selectivity for the same desired target cell, thereby significantly increasing the selectivity for the desired target compared to each individual antibody. Exemplary embodiments of a TetBiAb containing Fabs that bind any of the disease-specific antigens paired with another Fab that binds a second disease-specific antigen on the same target cell are, for example, anti-Her2/anti-Her3 and anti-EGFR/anti-IGF-1R.

Alternatively, a TetBiAb is directed against any of the disease-specific antigens and against an antigen that is expressed by a class of normal cells. In one exemplary embodiment the TetBiAb is anti-EpCAM/anti-CD47. In yet further exemplary embodiments, a TetBiAb targets two different antigens that are expressed by a class of normal cells, such as anti-CD20/anti-CD47 or anti-CD20/anti-CD52. It yet further embodiments, TetBiAbs contain Fabs in which one or both Fabs bind to a soluble factor, such as any growth factor, e.g., EGF, HGF, VEGF, and CSF-1, or cytokine, e.g. IL-6, IL-10, IL-12 and TNFα.

In another aspect of the invention, the invention provides methods for administering the TetBiAb into subjects, preferably humans, for treatment of diseases such as cancer, inflammatory diseases, autoimmune diseases, and infections.

Methods of preparing and administering a tetravalent bispecific antibody of the invention to a subject are well known to or are readily determined by a person skilled in the art. The route of administration of the tetravalent bispecific antibodies may be oral, parenteral, topical or by inhalation. Examples of parenteral administration include intravenous, intraarterial, intraperitoneal, intramuscular, subcutaneous, rectal or vaginal administration. A preferred form for administration is, for example, a solution for injection, in particular for intravenous or intraarterial injection or drip. Usually, a suitable pharmaceutical composition for injection may further comprise a pharmaceutically acceptable carrier. Examples of pharmaceutically acceptable carrier include saline, sterile water, Ringer's solution, buffered saline, dextrose solution, maltodextrin solution, glycerol, ethanol, etc. Optionally, conventional additives, such as antioxidants, buffers, bacteriostatic agents, etc., may be added to the composition.

The effective dosage of a tetravalent bispecific antibody for the treatment of a patient depends on many different factors, including the route of administration, state of health of the patient, the severity of the disease, the patient's weight, age, and gender, etc. In general, it may administered as a single dose, a daily dose, a weekly dose, a weekly dose, a biweekly dose, a monthly dose, etc. The dose may range from 0.1 mg/kg to 100 mg/kg of the tetravalent bispecific antibody.

In an exemplary embodiment, an effective dose of the TetBiAb anti-CD20/anti-CD47, in the typical range of 1 to 10 mg/kg, is administered intravenously into patients suffering from a B cell disorder, for example, from non-Hodgkin's lymphomas, rheumatoid arthritis, or systemic lupus erythematosus.

In another exemplary embodiment, an effective dose of the tetravalent bispecific antibody anti-EGFR/anti-CD16, in the typical range of 1 to 10 mg/kg, is administered intravenously into patients with solid tumors overexpressing EGFR, such as a colorectal or a lung cancer.

In the treatment of cancer, a tetravalent bispecific antibody may be used in conjunction or in combination with any chemotherapeutic agent or regimen that eliminates, reduces, or controls the growth of neoplastic cells in the patient. Exemplary chemotherapeutic agents include an alkylating agent, a vinca alkaloid, a taxane, an antimetabolite, a nitrosourea agent, a topoisomerase inhibitor, an aromatase inhibitor, a P-glycoprotein inhibitor, a platinum complex derivative, a hormone antagonist, a cytotoxic antibiotic, etc. The amount of chemotherapeutic agent to be used in combination with the tetravalent bispecific antibody may vary by subject and type and severity of disease and may be administered according to what is known in the art. See, for example, Chabner et al., Antineoplastic Agents, in Goodman & Gilman's The Pharmacological Basis of Therapeutics 1233-1287 (Joel G. Hardman et al., eds., 9^{th} ed. 1996).

Other advantages and features of the invention will be apparent from the examples, drawings, and claims that follow.

### EXAMPLES

The following examples serve to illustrate certain preferred embodiments and aspects of the present invention and are not to be construed as limiting the scope thereof.

Unless otherwise noted, the numbering of the amino acid residues in an IgG heavy chain is that of the EU index as in Kabat et al, Sequences of Proteins of Immunological Interest, 5^{th} Ed., Public Health Service, NIH, Bethesda, Md. (1991).

Table 1 provides sequences described herein. All polypeptide sequences of secreted molecules are shown without signal sequence. Variable domain is underlined.

| SEQ | | |
|---|---|---|
| ID | | |
| NO: | Description | Sequence |
| 1 | anti-EGFR | GACATCTTGCTGACTCAGTCTCCAGTCATCCTGTCTGTGAGTCCAGGAGAAAGAGTCAGTTTCTCCT |
| | Fab light | GCAGGGCCAGTCAGAGTATTGGCACAAACATACACTGGTATCAGCAAAGAACAAATGGTTCTCCAAG |
| | chain | GCTTCTCATAAAGTATGCTTCTGAGTCTATCTCTGGAATCCCTTCCAGGTTTAGTGGCAGTGGATCA |
| | | GGGACAGATTTTACTCTTAGCATCAACAGTGTGGAGTCTGAAGATATTGCAGATTATTACTGTCAAC |
| | | AAAATAATAACTGGCCAACCACGTTCGGTGCTGGGACCAAGCTGGAGCTGAAAACTGTGGCTGCACC |
| | | ATCTGTCTTCATCTTCCCGCCATCTGATGAGCAGTTGAAATCTGGAACTGCCTCTGTTGTGTGCCTG |
| | | CTGAATAACTTCTATCCCAGAGAGGCCAAAGTACAGTGGAAGGTGGATAACGCCCTCCAATCGGGTA |
| | | ACTCCCAGGAGAGTGTCACAGAGCAGGACAGCAAGGACAGCACCTACAGCCTCAGCAGCACCCTGAC |
| | | GCTGAGCAAAGCAGACTACGAGAAACACAAAGTCTACGCCTGCGAAGTCACCCATCAGGGCCTGAGC |
| | | TCGCCCGTCACAAAGAGCTTCAACAGGGGAGAG |
| 2 | anti-EGFR | DILLTQSPVILSVSPGERVSFSCRASQSIGTNIHWYQQRTNGSPRLLIKYASESISGIPSRFSGSGS |
| | Fab light | GTDFTLSINSVESEDIADYYCQQNNNWPTTFGAGTKLELKRTVAAPSVFIFPPSDEQLKSGTASVVC |
| | chain | LLNNFYPREAKVQWKVDNALQSGNSQESVTEQDSKDSTYSLSSTLTLSKADYEKHKVYACEVTHQGL |
| | | SSPVTKSFNRGEC |
| 3 | anti-EGFR | caggtgcagctgaagcagtcaggacctggcctagtgcagccctcacagagcctgtccatcacctgca |
| | Fab heavy | cagtctctggtttctcattaactaactatggtgtacactgggttcgccagtctccaggaaagggtct |
| | chain | ggagtggctgggagtgatatggagtggtggaaacacagactataatacacctttcacatccagactg |
| | | agcatcaacaaggacaattccaagagccaagttttctttaaaatgaacagtctgcaatctaatgaca |
| | | cagccatatattactgtgccagagccctcacctactatgattacgagtttgcttactggggccaagg |
| | | gactctggtcactgtctctgcaGCTAGCACCAAGGGCCCATCGGTCTTCCCCCTGGCACCCTCCTCC |
| | | AAGAGCACCTCTGGGGGCACAGCGGCCCTGGGCTGCCTGGTCAAGGACTACTTCCCCGAACCGGTGA |
| | | CGGTGTCGTGGAACTCAGGCGCCCTGACCAGCGGCGTGCACACCTTCCCGGCTGTCCTACAGTCCTC |
| | | AGGACTCTACTCCCTCAGCAGCGTGGTGACCGTGCCCTCCAGCAGCTTGGGCACCCAGACCTACATC |
| | | TGCAACGTGAATCACAAGCCCAGCAACACCAAGGTGGACAAGAGAGTTGAGCCCAAATCTTGT |
| 4 | anti-EGFR | QVQLKQSGPGLVQPSQSLSITCTVSGFSLTNYGVHWVRQSPGKGLEWLGVIWSGGNTDYNTPFTSRL |
| | Fab heavy | SINKDNSKSQVFFKMNSLQSNDTAIYYCARALTYYDYEFAYWGQGTLVTVSAASTKGPSVFPLAPSS |
| | chain | KSTSGGTAALGCLVKDYFPEPVTVSWNSGALTSGVHTFPAVLQSSGLYSLSSVVTVPSSSLGTQTYI |
| | | CNVNHKPSNTKVDKKVEPKSC |
| 5 | linker | ggtggaggtgggagc |
| 6 | linker | GGGGS |
| 7 | mutated | GAGCCCAAATCTTCT |
| | hinge | |
| | region | |
| 8 | mutated | EPKSS |
| | hinge | |
| | region | |
| 9 | hinge | GAGCCCAAATCTTGT |
| | region | |
| 10 | hinge | EPKSC |
| | region H* | |
| 11 | H-CH2-CH3- | atgaagcttcctgttaggctgttggtgctgatgttctggatccctgctagcttaagcgagcccaaat |
| | L-VH(anti- | cttctgacaaaactcacacatgcccaccgtgcccagcacctgaactcctgggggggccctcagtctt |
| | EGFR)-CH1- | cctcttccccccaaaacccaaggacaccctcatgatctctagaacccctgaggtcacatgcgtggtg |
| | H* (L = | gtggacgtgagccacgaagaccctgaggtcaagttcaactggtacgtggacggcgtggaggtgcata |
| | G4S) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggtggaggtgggagcCAGGTGCAGCTGAAGCAGTCAGGACCTGGCCTAGTG |
| | | CAGCCCTCACAGAGCCTGTCCATCACCTGCACAGTCTCTGGTTTCTCATTAACTAACTATGGTGTAC |
| | | ACTGGGTTCGCCAGTCTCCAGGAAAGGGTCTGGAGTGGCTGGGAGTGATATGGAGTGGTGGAAACAC |
| | | AGACTATAATACACCTTTCACATCCAGACTGAGCATCAACAAGGACAATTCCAAGAGCCAAGTTTTC |
| | | TTTAAAATGAACAGTCTGCAATCTAATGACACAGCCATATATTACTGTGCCAGAGCCCTCACCTACT |
| | | ATGATTACGAGTTTGCTTACTGGGGCCAAGGGACTCTGGTCACTGTCTCTGCAGCCTCCACCAAGGG |
| | | CCCATCGGTCTTCCCCCTGGCACCCTCCTCCAAGAGCACCTCTGGGGGCACAGCGGCCCTGGGCTGC |
| | | CTGGTCAAGGACTACTTCCCCGAACCGGTGACGGTGTCGTGGAACTCAGGCGCCCTGACCAGCGGCG |
| | | TGCACACCTTCCCGGCTGTCCTACAGTCCTCAGGACTCTACTCCCTCAGCAGCGTGGTGACCGTGCC |
| | | CTCCAGCAGCTTGGGCACCCAGACCTACATCTGCAACGTGAATCACAAGCCCAGCAACACCAAGGTG |
| | | GACAAGAAAGTTGAGCCCAAATCTTGTTGA |
| 12 | VL(anti- | ATGGAGTTGCCTGTTAGGCTGTTGGTGCTGATGTTCTGGATTCCTGCTTCCTTAAGCGACATCTTGC |
| | EGFR)-CL | TGACTCAGTCTCCAGTCATCCTGTCTGTGAGTCCAGGAGAAAGAGTCAGTTTCTCCTGCAGGGCCAG |
| | | TCAGAGTATTGGCACAAACATACACTGGTATCAGCAAAGAACAAATGGTTCTCCAAGGCTTCTCATA |
| | | AAGTATGCTTCTGAGTCTATCTCTGGAATCCCTTCCAGGTTTAGTGGCAGTGGATCAGGGACAGATT |
| | | TTACTCTTAGCATCAACAGTGTGGAGTCTGAAGATATTGCAGATTATTACTGTCAACAAAATAATAA |
| | | CTGGCCAACCACGTTCGGTGCTGGGACCAAGCTGGAGCTGAAACGAACTGTGGCTGCACCATCTGTC |
| | | TTCATCTTCCCGCCATCTGATGAGCAGTTGAAATCTGGAACTGCCTCTGTTGTGTGCCTGCTGAATA |
| | | ACTTCTATCCCAGAGAGGCCAAAGTACAGTGGAAGGTGGATAACGCCCTCCAATCGGGTAACTCCCA |
| | | GGAGAGTGTCACAGAGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGCCTGAGCTCGCCCG |
| | | TCACAAAGAGCTTCAACAGGGGAGAGTGTTAG |
| 13 | H-CH2-CH3- | EPKSSDKTHTCPPCPAPPVAGPSVFLFPPKPKDTLMISRTPEVTCVVVDVSHEDPEVKFNWYVDGVE |
| | L-VH(anti- | VHNAKTKPREEQYNSTYRVVSVLTVLHQDWLNGKEYKCKVSNKALPSSIEKTISKAKGQPREPQVYT |
| | EGFR)-CH1- | LPPSREEMTKNQVSLTCLVKGFYPSDIAVEWESNGQPENNYKTTPPVLDSDGSFFLYSKLTVDKSRW |
| | H* (L = | QQGNVFSCSVMHEALHNHYTQKSLSLSPGAGGGGSQVQLKQSGPGLVQPSQSLSITCTVSGFSLTNY |
| | G4S) | GVHWVRQSPGKGLEWLGVIWSGGNTDYNTPFTSRLSINKDNSKSQVFFKMNSLQSNDTAIYYCARAL |
| | | TYYDYEFAYWGQGTLVTVSAASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKRVEPKSC |
| 14 | VL(anti- | DILLTQSPVILSVSPGERVSFSCRASQSIGTNIHWYQQRTNGSPRLLIKYASESISGIPSRFSGSGS |
| | EGFR)-CL | GTDFTLSINSVESEDIADYYCQQNNNWPTTFGAGTKLELKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 15 | CH2-CH3- | atgaagcttcctgttaggctgttggtgctgatgttctggatccctgctagcttaagcgagcccaaat |
| | L-VL(anti- | cttctgacaaaactcacacatgcccaccgtgcccagcacctgaactcctgggggggccctcagtctt |
| | EGFR)-CL | cctcttccccccaaaacccaaggacaccctcatgatctctagaacccctgaggtcacatgcgtggtg |
| | (L = G4S) | gtggacgtgagccacgaagaccctgaggtcaagttcaactggtacgtggacggcgtggaggtgcata |
| | | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggtggaggtgggagcGACATCTTGCTGACTCAGTCTCCAGTCATCCTGTCT |
| | | GTGAGTCCAGGAGAAAGAGTCAGTTTCTCCTGCAGGGCCAGTCAGAGTATTGGCACAAACATACACT |
| | | GGTATCAGCAAAGAACAAATGGTTCTCCAAGGCTTCTCATAAAGTATGCTTCTGAGTCTATCTCTGG |
| | | AATCCCTTCCAGGTTTAGTGGCAGTGGATCAGGGACAGATTTTACTCTTAGCATCAACAGTGTGGAG |
| | | TCTGAAGATATTGCAGATTATTACTGTCAACAAAATAATAACTGGCCAACCACGTTCGGTGCTGGGA |
| | | CCAAGCTGGAGCTGAAACGAACTGTGGCTGCACCATCTGTCTTCATCTTCCCGCCATCTGATGAGCA |
| | | GTTGAAATCTGGAACTGCCTCTGTTGTGTGCCTGCTGAATAACTTCTATCCCAGAGAGGCCAAAGTA |
| | | CAGTGGAAGGTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAAACACAAAGT |
| | | CTACGCCTGCGAAGTCACCCATCAGGGCCTGAGCTCGCCCGTCACAAAGAGCTTCAACAGGGGAGAG |
| | | TGTTAG |
| 16 | VH(ant- | ATGGAGTTGCCTGTTAGGCTGTTGGTGCTGATGTTCTGGATTCCTGCTTCCAGCAGCcaggtgcagc |
| | EGFR)-CH1- | tgaagcagtcaggacctggcctagtgcagccctcacagagcctgtccatcacctgcacagtctctgg |
| | H* | tttctcattaactaactatggtgtacactgggttcgccagtctccaggaaagggtctggagtggctg |
| | | ggagtgatatggagtggtggaaacacagactataatacacctttcacatccagactgagcatcaaca |
| | | aggacaattccaagagccaagttttctttaaaatgaacagtctgcaatctaatgacacagccatata |
| | | ttactgtgccagagccctcacctactatgattacgagtttgcttactggggccaagggactctggtc |
| | | actgtctctgcaGCTAGCACCAAGGGCCCATCGGTCTTCCCCCTGGCACCCTCCTCCAAGAGCACCT |
| | | CTGGGGGCACAGCGGCCCTGGGCTGCCTGGTCAAGGACTACTTCCCCGAACCGGTGACGGTGTCGTG |
| | | GAACTCAGGCGCCCTGACCAGCGGCGTGCACACCTTCCCGGCTGTCCTACAGTCCTCAGGACTCTAC |
| | | TCCCTCAGCAGCGTGGTGACCGTGCCCTCCAGCAGCTTGGGCACCCAGACCTACATCTGCAACGTGA |
| | | ATCACAAGCCCAGCAACACCAAGGTGGACAAGAGAGTTGAGCCCAAATCTTGTTAG |
| 17 | H-CH2-CH3- | EPKSSDKTHTCPPCPAPPVAGPSVFLFPPKPKDTLMISRTPEVTCVVVDVSHEDPEVKFNWYVDGVE |
| | L-VL(anti- | VHNAKTKPREEQYNSTYRVVSVLTVLHQDWLNGKEYKCKVSNKALPSSIEKTISKAKGQPREPQVYT |
| | EGFR)-CL | LPPSREEMTKNQVSLTCLVKGFYPSDIAVEWESNGQPENNYKTTPPVLDSDGSFFLYSKLTVDKSRW |
| | (L = G4S) | QQGNVFSCSVMHEALHNHYTQKSLSLSPGAGGGGSDILLTQSPVILSVSPGERVSFSCRASQSIGTN |
| | | IHWYQQRTNGSPRLLIKYASESISGIPSRFSGSGSGTDFTLSINSVESEDIADYYCQQNNNWPTTFG |
| | | AGTKLELKRTVAAPSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVIKSFNRGEC |
| 18 | VH(anti- | QVQLKQSGPGLVQPSQSLSITCTVSGFSLTNYGVHWVRQSPGKGLEWLGVIWSGGNTDYNTPFTSRL |
| | EGFR)-CH1- | SINKDNSKSQVFFKMNSLQSNDTAIYYCARALTYYDYEFAYWGQGTLVTVSAASTKGPSVFPLAPSS |
| | H* | KSTSGGTAALGCLVKDYFPEPVTVSWNSGALTSGVHTFPAVLQSSGLYSLSSVVTVPSSSLGTQTYI |
| | | CNVNHKPSNTKVDKKVEPKSC |
| 19 | H-CH2-CH3- | atgaagcttcctgttaggctgttggtgctgatgttctggatccctgctagcttaagcgagcccaaat |
| | L-VL(anti- | cttctgacaaaactcacacatgcccaccgtgcccagcacctgaactcctgggggggccctcagtctt |
| | EGFR)-CL | cctcttccccccaaaacccaaggacaccctcatgatctctagaacccctgaggtcacatgcgtggtg |
| | (L = | gtggacgtgagccacgaagaccctgaggtcaagttcaactggtacgtggacggcgtggaggtgcata |
| | (G4S)4) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggcggcggaggaagcggaggaggtggcagcggtggcggtggctccggcgga |
| | | ggtggctccggaGACATCTTGCTGACTCAGTCTCCAGTCATCCTGTCTGTGAGTCCAGGAGAAAGAG |
| | | TCAGTTTCTCCTGCAGGGCCAGTCAGAGTATTGGCACAAACATACACTGGTATCAGCAAAGAACAAA |
| | | TGGTTCTCCAAGGCTTCTCATAAAGTATGCTTCTGAGTCTATCTCTGGAATCCCTTCCAGGTTTAGT |
| | | GGCAGTGGATCAGGGACAGATTTTACTCTTAGCATCAACAGTGTGGAGTCTGAAGATATTGCAGATT |
| | | ATTACTGTCAACAAAATAATAACTGGCCAACCACGTTCGGTGCTGGGACCAAGCTGGAGCTGAAACG |
| | | AACTGTGGCTGCACCATCTGTCTTCATCTTCCCGCCATCTGATGAGCAGTTGAAATCTGGAACTGCC |
| | | TCTGTTGTGTGCCTGCTGAATAACTTCTATCCCAGAGAGGCCAAAGTACAGTGGAAGGTGGATAACG |
| | | CCCTCCAATCGGGTAACTCCCAGGAGAGTGTCACAGAGCAGGACAGCAAGGACAGCACCTACAGCCT |
| | | CAGCAGCACCCTGACGCTGAGCAAAGCAGACTACGAGAAACACAAAGTCTACGCCTGCGAAGTCACC |
| | | CATCAGGGCCTGAGCTCGCCCGTCACAAAGAGCTTCAACAGGGGAGAGTGTTAG |
| 20 | H-CH2-CH3- | EPKSSDKTHTCPPCPAPPVAGPSVFLFPPKPKDTLMISRTPEVTCVVVDVSHEDPEVKFNWYVDGVE |
| | L-VL(anti- | VHNAKTKPREEQYNSTYRVVSVLTVLHQDWLNGKEYKCKVSNKALPSSIEKTISKAKGQPREPQVYT |
| | EGFR)-CL | LPPSREEMTKNQVSLTCLVKGFYPSDIAVEWESNGQPENNYKTTPPVLDSDGSFFLYSKLTVDKSRW |
| | (L = | QQGNVFSCSVMHEALHNHYTQKSLSLSPGAGGGGSGGGGSGGGGSGGGGSGDILLTQSPVILSVSPG |
| | (G4S)4) | ERVSFSCRASQSIGTNIHWYQQRTNGSPRLLIKYASESISGIPSRFSGSGSGTDFTLSINSVESEDI |
| | | ADYYCQQNNNWPTTFGAGTKLELKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQGLSSPVTKSFNRGEC |
| 21 | anti-CD20 | CAAATTGTTCTCTCCCAGTCTCCAGCAATCCTGTCTGCATCTCCAGGGGAGAAGGTCACAATGACTT |
| | Fab light | GCAGGGCCAGCTCAAGTGTAAGTTACATCCACTGGTTCCAGCAGAAGCCAGGTTCCTCCCCCAAACC |
| | chain | CTGGATTTATGCCACATCCAACCTGGCTTCTGGAGTCCCTGTTCGCTTCAGTGGCAGTGGGTCTGGG |
| | | ACTTCTTACTCTCTCACCATCAGCAGAGTGGAGGCTGAAGATGCTGCCACTTATTACTGCCAGCAGT |
| | | GGACTAGTAACCCACCCACGTTCGGAGGGGGGACCAAGCTGGAAATCAAAACTGTGGCTGCACCATC |
| | | TGTCTTCATCTTCCCGCCATCTGATGAGCAGTTGAAATCTGGAACTGCCTCTGTTGTGTGCCTGCTG |
| | | AATAACTTCTATCCCAGAGAGGCCAAAGTACAGTGGAAGGTGGATAACGCCCTCCAATCGGGTAACT |
| | | CCCAGGAGAGTGTCACAGAGCAGGACAGCAAGGACAGCACCTACAGCCTCAGCAGCACCCTGACGCT |
| | | GAGCAAAGCAGACTACGAGAAACACAAAGTCTACGCCTGCGAAGTCACCCATCAGGGCCTGAGCTCG |
| | | CCCGTCACAAAGAGCTTCAACAGGGGAGAG |
| 22 | anti-CD20 | QIVLSQSPAILSASPGEKVTMTCRASSSVSYIHWFQQKPGSSPKPWIYATSNLASGVPVRFSGSGSG |
| | Fab light | TSYSLTISRVEAEDAATYYCQQWTSNPPTFGGGTKLEIKRTVAAPSVFIFPPSDEQLKSGTASVVCL |
| | chain | LNNFYPREAKVQWKVDNALQSGNSQESVTEQDSKDSTYSLSSTLTLSKADYEKHKVYACEVTHQGLS |
| | | SPVTKSFNRGEC |
| 23 | anti-CD20 | caggtacaactgcaacagcctggggctgagctggtgaagcctggggcctcagtgaagatgtcctgca |
| | Fab heavy | aggcttctggctacacatttaccagttacaatatgcactgggtaaaacagacacctggtcggggcct |
| | chain | ggaatggattggagctatttatcccggaaatggtgatacttcctacaatcagaagttcaaaggcaag |
| | | gccacattgactgctgacaaatcctccagcacagcctacatgcagctcagcagcctgacatctgagg |
| | | actctgcggtctattactgtgcaagatcgacttactacggcggtgactggtacttcaatgtctgggg |
| | | cgcagggaccacggtcaccgtctccgcaGCTAGCACCAAGGGCCCATCGGTCTTCCCCCTGGCACCC |
| | | TCCTCCAAGAGCACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTTGAGCCCAAATCT |
| 24 | anti-CD20 | QVQLQQPGAELVKPGASVKMSCKASGYTFTSYNMHWVKQTPGRGLEWIGAIYPGNGDTSYNQKFKGK |
| | Fab heavy | ATLTADKSSSTAYMQLSSLTSEDSAVYYCARSTYYGGDWYFNVWGAGTTVTVSAASTKGPSVFPLAP |
| | chain | SSKSTSGGTAALGCLVKDYFPEPVTVSWNSGALTSGVHTFPAVLQSSGLYSLSSVVTVPSSSLGTQT |
| | | YICNVNHKPSNTKVDKKVEPKSC |
| 25 | H-CH2-CH3- | atgaagcttcctgttaggctgttggtgctgatgttctggatccctgctagcttaagcgagcccaaat |
| | L-VH(anti- | cttctgacaaaactcacacatgcccaccgtgcccagcacctgaactcctgggggggccctcagtctt |
| | CD20)-CH1- | cctcttccccccaaaacccaaggacaccctcatgatctctagaacccctgaggtcacatgcgtggtg |
| | H* (L = | gtggacgtgagccacgaagaccctgaggtcaagttcaactggtacgtggacggcgtggaggtgcata |
| | G4S) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggtggaggtgggagccaggtacaactgcaacagcctggggctaagctaatg |
| | | aagcctggggcctcagtgaagatgtcctgcaaggcttctggctacacatttaccagttacaatatgc |
| | | actgggtaaaacagacacctggtcggggcctggaatggattggagctatttatcccggaaatggtga |
| | | tacttcctacaatcagaagttcaaaggcaaggccacattgactgctgacaaatcctccagcacagcc |
| | | tacatgcagctcagcagcctgacatctgaggactctgcggtctattactgtgcaagatcgacttact |
| | | acggcggtgactggtacttcaatgtctggggcgcagggaccacggtcaccgtctccgcaGCCTCCAC |
| | | CAAGGGCCCATCGGTCTTCCCCCTGGCACCCTCCTCCAAGAGCACCTCTGGGGGCACAGCGGCCCTG |
| | | GGCTGCCTGGTCAAGGACTACTTCCCCGAACCGGTGACGGTGTCGTGGAACTCAGGCGCCCTGACCA |
| | | GCGGCGTGCACACCTTCCCGGCTGTCCTACAGTCCTCAGGACTCTACTCCCTCAGCAGCGTGGTGAC |
| | | CGTGCCCTCCAGCAGCTTGGGCACCCAGACCTACATCTGCAACGTGAATCACAAGCCCAGCAACACC |
| | | AAGGTGGACAAGAAAGTTGAGCCCAAATCTTGTTGA |
| 26 | VL(anti- | ATGGAGTTGCCTGTTAGGCTGTTGGTGCTGATGTTCTGGATTCCTGCTTCCTTAAGCCAAATTGTTC |
| | CD20)-CL | TCTCCCAGTCTCCAGCAATCCTGTCTGCATCTCCAGGGGAGAAGGTCACAATGACTTGCAGGGCCAG |
| | | CTCAAGTGTAAGTTACATCCACTGGTTCCAGCAGAAGCCAGGTTCCTCCCCCAAACCCTGGATTTAT |
| | | GCCACATCCAACCTGGCTTCTGGAGTCCCTGTTCGCTTCAGTGGCAGTGGGTCTGGGACTTCTTACT |
| | | CTCTCACCATCAGCAGAGTGGAGGCTGAAGATGCTGCCACTTATTACTGCCAGCAGTGGACTAGTAA |
| | | CCCACCCACGTTCGGAGGGGGGACCAAGCTGGAAATCAAAACTGTGGCTGCACCATCTGTCTTCATC |
| | | TTCCCGCCATCTGATGAGCAGTTGAAATCTGGAACTGCCTCTGTTGTGTGCCTGCTGAATAACTTCT |
| | | ATCCCAGAGAGGCCAAAGTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCCTGACGCTGAGCAAAGCA |
| | | GACTACGAGAAACACAAAGTCTACGCCTGCGAAGTCACCCATCAGGGCCTGAGCTCGCCCGTCACAA |
| | | AGAGCTTCAACAGGGGAGAGTGT |
| 27 | H-CH2-CH3- | EPKSSDKTHTCPPCPAPELLGGPSVFLFPPKPKDTLMISRTPEVTCVVVDVSHEDPEVKFNWYVDGV |
| | L-VH(anti- | EVHNAKTKPREEQYNSTYRVVSVLTVLHQDWLNGKEYKCKVSNKALPAPIEKTISKAKGQPREPQVY |
| | CD20)-CH1- | TLPPSRDELTKNQVSLTCLVKGFYPSDIAVEWESNGQPENNYKTTPPVLDSDGSFFLYSKLTVDKSR |
| | H* (L = | WQQGNVFSCSVMHEALHNHYTQKSLSLSPGAGGGGSQVQLQQPGAELVKPGASVKMSCKASGYTFTS |
| | G4S) | YNMHWVKQTPGRGLEWIGAIYPGNGDTSYNQKFKGKATLTADKSSSTAYMQLSSLTSEDSAVYYCAR |
| | | STYYGGDWYFNVWGAGTTVTVSAASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKPSNTKVDKKVEPKSC |
| 28 | VL(ant- | QIVLSQSPAILSASPGEKVTMTCRASSSVSYIHWFQQKPGSSPKPWIYATSNLASGVPVRFSGSGSG |
| | CD20)-CL | TSYSLTISRVEAEDAATYYCQQWTSNPPTFGGGTKLEIKRGTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 29 | H-CH2-CH3- | atgaagcttcctgttaggctgttggtgctgatgttctggatccctgctagcttaagcgagcccaaat |
| | L-VH(anti- | cttctgacaaaactcacacatgcccaccgtgcccagcacctgaactcctgggggggccctcagtctt |
| | CD20)-CH1- | cctcttccccccaaaacccaaggacaccctcatgatctctagaacccctgaggtcacatgcgtggtg |
| | H* (L = | gtggacgtgagccacgaagaccctgaggtcaagttcaactggtacgtggacggcgtggaggtgcata |
| | (G4S)4) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggcggcggaggaagcggaggaggtggcagcggtggcggtggctccggcgga |
| | | ggtggctccggacaggtacaactgcaacagcctggggctgagctggtgaagcctggggcctcagtga |
| | | agatgtcctgcaaggcttctggctacacatttaccagttacaatatgcactgggtaaaacagacacc |
| | | tggtcggggcctggaatggattggagctatttatcccggaaatggtgatacttcctacaatcagaag |
| | | ttcaaaggcaaggccacattgactgctgacaaatcctccagcacagcctacatgcagctcagcagcc |
| | | tgacatctgaggactctgcggtctattactgtgcaagatcgacttactacggcggtgactggtactt |
| | | caatgtctggggcgcagggaccacggtcaccgtctccgcaGCCTCCACCAAGGGCCCATCGGTCTTC |
| | | CCCCTGGCACCCTCCTCCAAGAGCACCTCTGGGGGCACAGCGGCCCTGGGCTGCCTGGTCAAGGACT |
| | | ACTTCCCCGAACCGGTGACGGTGTCGTGGAACTCAGGCGCCCTGACCAGCGGCGTGCACACCTTCCC |
| | | GGCTGTCCTACAGTCCTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAGGTGGACAAGAAAGTTG |
| | | AGCCCAAATCTTGTTGA |
| 30 | H-CH2-CH3- | EPKSSDKTHTCPPCPAPELLGGPSVFLFPPKPKDTLMISRTPEVTCVVVDVSHEDPEVKFNWYVDGV |
| | L-VH(anti- | EVHNAKTKPREEQYNSTYRVVSVLTVLHQDWLNGKEYKCKVSNKALPAPIEKTISKAKGQPREPQVY |
| | CD20)-CH1- | TLPPSRDELTKNQVSLTCLVKGFYPSDIAVEWESNGQPENNYKTTPPVLDSDGSFFLYSKLTVDKSR |
| | H* (L = | WQQGNVFSCSVMHEALHNHYTQKSLSLSPGAGGGGSGGGGSGGGGSGGGGSGQVQLQQPGAELVKPG |
| | (G4S)4) | ASVKMSCKASGYTFTSYNMHWVKQTPGRGLEWIGAIYPGNGDTSYNQKFKGKATLTADKSSSTAYMQ |
| | | LSSLTSEDSAVYYCARSTYYGGDWYFNVWGAGTTVTVSAASTKGPSVFPLAPSSKSTSGGTAALGCL |
| | | VKDYFPEPVTVSWNSGALTSGVHTFPAVLQSSGLYSLSSVVTVPSSSLGTQTYICNVNHKPSNTKVD |
| | | KKVEPKSC |
| 31 | H-CH2-CH3- | atgaagcttcctgttaggctgttggtgctgatgttctggatccctgctagcttaagcgagcccaaat |
| | L-VL(anti- | cttctgacaaaactcacacatgcccaccgtgcccagcacctgaactcctgggggggccctcagtctt |
| | CD20)-CL | cctcttccccccaaaacccaaggacaccctcatgatctctagaacccctgaggtcacatgcgtggtg |
| | (L = G4S) | gtggacgtgagccacgaagaccctgaggtcaagttcaactggtacgtggacggcgtggaggtgcata |
| | | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggtggaggtgggagcCAAATTGTTCTCTCCCAGTCTCCAGCAATCCTGTCT |
| | | GCATCTCCAGGGGAGAAGGTCACAATGACTTGCAGGGCCAGCTCAAGTGTAAGTTACATCCACTGGT |
| | | TCCAGCAGAAGCCAGGTTCCTCCCCCAAACCCTGGATTTATGCCACATCCAACCTGGCTTCTGGAGT |
| | | CCCTGTTCGCTTCAGTGGCAGTGGGTCTGGGACTTCTTACTCTCTCACCATCAGCAGAGTGGAGGCT |
| | | GAAGATGCTGCCACTTATTACTGCCAGCAGTGGACTAGTAACCCACCCACGTTCGGAGGGGGGACCA |
| | | AGCTGGAAATCAAACGAACTGTGGCTGCACCATCTGTCTTCATCTTCCCGCCATCTGATGAGCAGTT |
| | | GAAATCTGGAACTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCAGGACAGCAAGG |
| | | ACAGCACCTACAGCCTCAGCAGCACCCTGACGCTGAGCAAAGCAGACTACGAGAAACACAAAGTCTA |
| | | CGCCTGCGAAGTCACCCATCAGGGCCTGAGCTCGCCCGTCACAAAGAGCTTCAACAGGGGAGAGTGT |
| | | TAG |
| 32 | VH(anti- | ATGGAGTTGCCTGTTAGGCTGTTGGTGCTGATGTTCTGGATTCCTGCTTCCAGCAGCCAGGTACAAC |
| | CD20)-CH1- | TGCAACAGCCTGGGGCTGAGCTGGTGAAGCCTGGGGCCTCAGTGAAGATGTCCTGCAAGGCTTCTGG |
| | H* | CTACACATTTACCAGTTACAATATGCACTGGGTAAAACAGACACCTGGTCGGGGCCTGGAATGGATT |
| | | GGAGCTATTTATCCCGGAAATGGTGATACTTCCTACAATCAGAAGTTCAAAGGCAAGGCCACATTGA |
| | | CTGCTGACAAATCCTCCAGCACAGCCTACATGCAGCTCAGCAGCCTGACATCTGAGGACTCTGCGGT |
| | | CTATTACTGTGCAAGATCGACTTACTACGGCGGTGACTGGTACTTCAATGTCTGGGGCGCAGGGACC |
| | | ACGGTCACCGTCTCCGCAGCTAGCACCAAGGGCCCATCGGTCTTCCCCCTGGCACCCTCCTCCAAGA |
| | | GCACCTCTGGGGGCACAGCGGCCCTGGGCTGCCTGGTCAAGGACTACTTCCCCGAACCGGTGACGGT |
| | | GTCGTGGAACTCAGGCGCCCTGACCAGCGGCGTGCACACCTTCCCGGCTGTCCTACAGTCCTCAGGA |
| | | CTCTACTCCCTCAGCAGCGTGGTGACCGTGCCCTCCAGCAGCTTGGGCACCCAGACCTACATCTGCG |
| | | CCGTGATCAGCAGCGGCGGCAGCTCCATCAACTACAAGAAAGTTGAGCCCAAATCTTGTTAA |
| 33 | H-CH2-CH3- | EPKSSDKTHTCPPCPAPPVAGPSVFLFPPKPKDTLMISRTPEVTCVVVDVSHEDPEVKFNWYVDGVE |
| | L-VL(anti- | VHNAKTKPREEQYNSTYRVVSVLTVLHQDWLNGKEYKCKVSNKALPSSIEKTISKAKGQPREPQVYT |
| | CD20)-CL | LPPSREEMTKNQVSLTCLVKGFYPSDIAVEWESNGQPENNYKTTPPVLDSDGSFFLYSKLTVDKSRW |
| | (L = G4S) | QQGNVFSCSVMHEALHNHYTQKSLSLSPGAGGGGSQIVLSQSPAILSASPGEKVTMTCRASSSVSYI |
| | | HWFQQKPGSSPKPWIYATSNLASGVPVRFSGSGSGTSYSLTISRVEAEDAATYYCQQWTSNPPTFGG |
| | | GTKLEIKRTVAAPSVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 34 | VH(anti- | QVQLQQPGAELVKPGASVKMSCKASGYTFTSYNMHWVKQTPGRGLEWIGAIYPGNGDTSYNQKFKGK |
| | CD20)CH1- | ATLTADKSSSTAYMQLSSLTSEDSAVYYCARSTYYGGDWYFNVWGAGTTVTVSAASTKGPSVFPLAP |
| | H* | SSKSTSGGTAALGCLVKDYFPEPVTVSWNSGALTSGVHTFPAVLQSSGLYSLSSVVTVPSSSLGTQT |
| | | YICNVNHKPSNTKVDKKVEPKSC |
| 35 | H-CH2-CH3- | atgaagcttcctgttaggctgttggtgctgatgttctggatccctgctagcttaagcgagcccaaat |
| | L-VL(anti- | cttctgacaaaactcacacatgcccaccgtgcccagcacctgaactcctgggggggccctcagtctt |
| | CD20)-CL | cctcttccccccaaaacccaaggacaccctcatgatctctagaacccctgaggtcacatgcgtggtg |
| | (L = | gtggacgtgagccacgaagaccctgaggtcaagttcaactggtacgtggacggcgtggaggtgcata |
| | (G4S)4) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggcggcggaggaagcggaggaggtggcagcggtggcggtggctccggcgga |
| | | ggtggctccggaCAAATTGTTCTCTCCCAGTCTCCAGCAATCCTGTCTGCATCTCCAGGGGAGAAGG |
| | | TCACAATGACTTGCAGGGCCAGCTCAAGTGTAAGTTACATCCACTGGTTCCAGCAGAAGCCAGGTTC |
| | | CTCCCCCAAACCCTGGATTTATGCCACATCCAACCTGGCTTCTGGAGTCCCTGTTCGCTTCAGTGGC |
| | | AGTGGGTCTGGGACTTCTTACTCTCTCACCATCAGCAGAGTGGAGGCTGAAGATGCTGCCACTTATT |
| | | ACTGCCAGCAGTGGACTAGTAACCCACCCACGTTCGGAGGGGGGACCAAGCTGGAAATCAAACGAAC |
| | | TGTGGCTGCACCATCTGTCTTCATCTTCCCGCCATCTGATGAGCAGTTGAAATCTGGAACTGCCTCT |
| | | GTTGTGTGCCTGCTGAATAACTTCTATCCCAGAGAGGCCAAAGTACAGTGGAAGGTGGATAACGCCC |
| | | TCCAATCGGGTAACTCCCAGGAGAGTGTCACAGAGCAGGACAGCAAGGACAGCACCTACAGCCTCAG |
| | | CAGCACCCTGACGCTGAGCAAAGCAGACTACGAGAAACACAAAGTCTACGCCTGCGAAGTCACCCAT |
| | | CAGGGCCTGAGCTCGCCCGTCACAAAGAGCTTCAACAGGGGAGAGTGTTAG |
| 36 | H-CH2-CH3- | EPKSSDKTHTCPPCPAPPVAGPSVFLFPPKPKDTLMISRTPEVTCVVVDVSHEDPEVKFNWYVDGVE |
| | L-VL(anti- | VHNAKTKPREEQYNSTYRVVSVLTVLHQDWLNGKEYKCKVSNKALPSSIEKTISKAKGQPREPQVYT |
| | CD20)-CL | LPPSREEMTKNQVSLTCLVKGFYPSDIAVEWESNGQPENNYKTTPPVLDSDGSFFLYSKLTVDKSRW |
| | (L = | QQGNVFSCSVMHEALHNHYTQKSLSLSPGAGGGGSGGGGSGGGGSGGGGSGQIVLSQSPAILSASPG |
| | (G4S)4) | EKVTMTCRASSSVSYIHWFQQKPGSSPKPWIYATSNLASGVPVRFSGSGSGTSYSLTISRVEAEDAA |
| | | TYYCQQWTSNPPTFGGGTKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSSPVTKSFNRGEC |
| 37 | anti-CD16 | gacattgtgctgacccaatctccagcttctttggctgtgtctctagggcagagggccaccatctcct |
| | Fab light | gcaaggccagccaaagtgttgattttgatggtgatagttttatgaactggtaccaacagaaaccagg |
| | chain | acagccacccaaactcctcatctatactacatccaatctagaatctggcatcccagccaggtttagt |
| | | gccagtgggtctgggacagacttcaccctcaacatccatcctgtggaggaggaggatactgcaacct |
| | | attactgtcagcaaagtaatgaggacccgtacacgttcggaggggggaccaagctggagctgaaaAC |
| | | TGTGGCTGCACCATCTGTCTTCATCTTCCCGCCATCTGATGAGCAGTTGAAATCTGGAACTGCCTCT |
| | | GTTGTGTGCCTGCTGAATAACTTCTATCCCAGAGAGGCCAAAGTACAGTGGAAGGTGGATAACGCCC |
| | | TCCAATCGGGTAACTCCCAGGAGAGTGTCACAGAGCAGGACAGCAAGGACAGCACCTACAGCCTCAG |
| | | CAGCACCCTGACGCTGAGCAAAGCAGACTACGAGAAACACAAAGTCTACGCCTGCGAAGTCACCCAT |
| | | CAGGGCCTGAGCTCGCCCGTCACAAAGAGCTTCAACAGGGGAGAG |
| 38 | anti-CD16 | DIVLTQSPASLAVSLGQRATISCKASQSVDFDGDSFMNWYQQKPGQPPKLLIYTTSNLESGIPARFS |
| | Fab light | ASGSGTDFTLNIHPVEEEDTATYYCQQSNEDPYTFGGGTKLELKRTVAAPSVFIFPPSDEQLKSGTA |
| | chain | SVVCLLNNFYPREAKVQWKVDNALQSGNSQESVTEQDSKDSTYSLSSTLTLSKADYEKHKVYACEVT |
| | | HQGLSSPVTKSFNRGEC |
| 39 | anti-CD16 | CAGGTACAACTGCAACAGCCTGGGGCTGAGCTGGTGAAGCCTGGGGCCTCAGTGAAGATGTCCTGCA |
| | Fab heavy | AGGCTTCTGGCTACACATTTACCAGTTACAATATGCACTGGGTAAAACAGACACCTGGTCGGGGCCT |
| | chain | GGAATGGATTGGAGCTATTTATCCCGGAAATGGTGATACTTCCTACAATCAGAAGTTCAAAGGCAAG |
| | | GCCACATTGACTGCTGACAAATCCTCCAGCACAGCCTACATGCAGCTCAGCAGCCTGACATCTGAGG |
| | | ACTCTGCGGTCTATTACTGTGCAAGATCGACTTACTACGGCGGTGACTGGTACTTCAATGTCTGGGG |
| | | CGCAGGGACCACGGTCACCGTCTCCGCAGCTAGCACCAAGGGCCCATCGGTCTTCCCCCTGGCACCC |
| | | TCCTCCAAGAGCACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAGAGTTGAGCCCAAATCTT |
| | | GT |
| 40 | anti-CD16 | QVTLKESGPGILQPSQTLSLTCSFSGFSLRTSGMGVGWIRQPSGKGLEWLAHIWWDDDKRYNPALKS |
| | Fab heavy | RLTISKDTSSNQVFLKIASVDTADTATYYCAQINPAWFAYWGQGTLVTVSAASTKGPSVFPLAPSSK |
| | chain | STSGGTAALGCLVKDYFPEPVTVSWNSGALTSGVHTFPAVLQSSGLYSLSSVVTVPSSSLGTQTYIC |
| | | NVNHKPSNTKVDKKVEPKSC |
| 41 | VH(anti- | atggagttgcctgttaggctgttggtgctgatgttctggattcctgctagctccagccaggtgcagc |
| | EGFR)-CH1- | tgaagcagtcaggacctggcctagtgcagccctcacagagcctgtccatcacctgcacagtctctgg |
| | H-CH2-CH3- | tttctcattaactaactatggtgtacactgggttcgccagtctccaggaaagggtctggagtggctg |
| | L-VL(anti- | ggagtgatatggagtggtggaaacacagactataatacacctttcacatccagactgagcatcaaca |
| | CD16)-CL | aggacaattccaagagccaagttttctttaaaatgaacagtctgcaatctaatgacacagccatata |
| | (L = G4S) | ttactgtgccagagccctcacctactatgattacgagtttgcttactggggccaagggactctggtc |
| | | actgtctctgcagcctccaccaagggcccatcggtcttccccctggcaccctcctccaagagcacct |
| | | ctgggggcacagcggccctgggctgcctggtcaaggactacttccccgaaccggtgacggtgtcgtg |
| | | gaactcaggcgccctgaccagcggcgtgcacaccttcccggctgtcctacagtcctcaggactctac |
| | | tccctcagcagcgtggtgaccgtgccctccagcagcttgggcacccagacctacatctgcaacgtga |
| | | atcacaagcccagcaacaccaaggtggacaagagagttgagcccaaatcttgtgacaaaactcacac |
| | | atgcccaccgtgcccagcacctccagtggccggaccgtcagtcttcctcttccccccaaaacccaag |
| | | gacaccctcatgatctcccggacccctgaggtcacatgcgtggtggtggacgtgagccacgaagacc |
| | | ctgaggtcaagttcaactggtacgtggacggcgtggaggtgcataatgccaagacaaagccgcggga |
| | | ggagcagtacaacagcacgtaccgtgtggtcagcgtcctcaccgtcctgcaccaggactggctgaat |
| | | ggcaaggagtacaagtgcaaggtctccaacaaagccctcccatccagcatcgagaaaaccatctcca |
| | | aagccaaagggcagccccgagaaccacaggtgtacaccctgcccccatcacgggaggagatgaccaa |
| | | gaaccaggtcagcctgacctgcctggtcaaaggcttctatcccagcgacatcgccgtggagtgggag |
| | | agcaatgggcagccggagaacaactacaagaccacgcctcccgtgctggactccgacggctccttct |
| | | tcctctatagcaagctcaccgtggacaagagcaggtggcagcaggggaacgtcttctcatgctccgt |
| | | gatgcatgaggctctgcacaaccactacacacagaagagcctctccctgtccccgggtgcaggtgga |
| | | ggtgggagcgacattgtgctgacccaatctccagcttctttggctgtgtctctagggcagagggcca |
| | | ccatctcctgcaaggccagccaaagtgttgattttgatggtgatagttttatgaactggtaccaaca |
| | | gaaaccaggacagccacccaaactcctcatctatactacatccaatctagaatctggcatcccagcc |
| | | aggtttagtgccagtgggtctgggacagacttcaccctcaacatccatcctgtggaggaggaggata |
| | | ctgcaacctattactgtcagcaaagtaatgaggacccgtacacgttcggaggggggaccaagctgga |
| | | gctgaaacgaactgtggctgcaccatctgtcttcatcttcccgccatctgatgagcagttgaaatct |
| | | ggaactgcctctgttgtgtgcctgctgaataacttctatcccagagaggccaaagtacagtggaagg |
| | | tggataacgccctccaatcgggtaactcccaggagagtgtcacagagcaggacagcaaggacagcac |
| | | ctacagcctcagcagcaccctgacgctgagcaaagcagactacgagaaacacaaagtctacgcctgc |
| | | gaagtcacccatcagggcctgagctcgcccgtcacaaagagcttcaacaggggagagtgttga |
| 42 | VH(anti- | ATGGAGTTGCCTGTTAGGCTGTTGGTGCTGATGTTCTGGATTCCTGCTTCCAGCAGCCAGGTACAAC |
| | CD16)-CH1- | TGCAACAGCCTGGGGCTGAGCTGGTGAAGCCTGGGGCCTCAGTGAAGATGTCCTGCAAGGCTTCTGG |
| | H* | CTACACATTTACCAGTTACAATATGCACTGGGTAAAACAGACACCTGGTCGGGGCCTGGAATGGATT |
| | | GGAGCTATTTATCCCGGAAATGGTGATACTTCCTACAATCAGAAGTTCAAAGGCAAGGCCACATTGA |
| | | CTGCTGACAAATCCTCCAGCACAGCCTACATGCAGCTCAGCAGCCTGACATCTGAGGACTCTGCGGT |
| | | CTATTACTGTGCAAGATCGACTTACTACGGCGGTGACTGGTACTTCAATGTCTGGGGCGCAGGGACC |
| | | ACGGTCACCGTCTCCGCAGCTAGCACCAAGGGCCCATCGGTCTTCCCCCTGGCACCCTCCTCCAAGA |
| | | GCACCTCTGGGGGCACAGCGGCCCTGGGCTGCCTGGTCAAGGACTACTTCCCCGAACCGGTGACGGT |
| | | GTCGTGGAACTCAGGCGCCCTGACCAGCGGCGTGCACACCTTCCCGGCTGTCCTACAGTCCTCAGGA |
| | | CTCTACTCCCTCAGCAGCGTGGTGACCGTGCCCTCCAGCAGCTTGGGCACCCAGACCTACATCTGCA |
| | | ACGTGAATCACAAGCCCAGCAACACCAAGGTGGACAAGAGAGTTGAGCCCAAATCTTGTTAG |
| 43 | VH(anti- | QVQLKQSGPGLVQPSQSLSITCTVSGFSLTNYGVHWVRQSPGKGLEWLGVIWSGGNTDYNTPFTSRL |
| | EGFR)-CH1- | SINKDNSKSQVFFKMNSLQSNDTAIYYCARALTYYDYEFAYWGQGTLVTVSAASTKGPSVFPLAPSS |
| | H-CH2-CH3- | KSTSGGTAALGCLVKDYFPEPVTVSWNSGALTSGVHTFPAVLQSSGLYSLSSVVTVPSSSLGTQTYI |
| | L-V(anti- | CNVNHKPSNTKVDKRVEPKSCDKTHTCPPCPAPPVAGPSVFLFPPKPKDTLMISRTPEVTCVVVDVS |
| | CD16)-CL | HEDPEVKFNWYVDGVEVHNAKTKPREEQYNSTYRVVSVLTVLHQDWLNGKEYKCKVSNKALPSSIEK |
| | (L = G4S) | TISKAKGQPREPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGAGGGGSDIVLTQSPASLAVSLG |
| | | QRATISCKASQSVDFDGDSFMNWYQQKPGQPPKLLIYTTSNLESGIPARFSASGSGTDFTLNIHPVE |
| | | EEDTATYYCQQSNEDPYTFGGGTKLELKRTVAAPSVFIFPPSDEQLKSGTASVVCLLNNFYPREAKV |
| | | QWKVDNALQSGNSQESVTEQDSKDSTYSLSSTLTLSKADYEKHKVYACEVTHQGLSSPVTKSFNRGE |
| | | C |
| 44 | VH(anti- | QVTLKESGPGILQPSQTLSLTCSFSGFSLRTSGMGVGWIRQPSGKGLEWLAHIWWDDDKRYNPALKS |
| | CD16)CH1- | RLTISKDTSSNQVFLKIASVDTADTATYYCAQINPAWFAYWGQGTLVTVSAASTKGPSVFPLAPSSK |
| | H* | STSGGTAALGCLVKDYFPEPVTVSWNSGALTSGVHTFPAVLQSSGLYSLSSVVTVPSSSLGTQTYIC |
| | | NVNHKPSNTKVDKRVEPKSC |
| 45 | VH(anti- | atggagttgcctgttaggctgttggtgctgatgttctggattcctgctagctccagcCAGGTACAAC |
| | CD16)-CH1- | TGCAACAGCCTGGGGCTGAGCTGGTGAAGCCTGGGGCCTCAGTGAAGATGTCCTGCAAGGCTTCTGG |
| | H-CH2-CH3- | CTACACATTTACCAGTTACAATATGCACTGGGTAAAACAGACACCTGGTCGGGGCCTGGAATGGATT |
| | L-VL(anti- | GGAGCTATTTATCCCGGAAATGGTGATACTTCCTACAATCAGAAGTTCAAAGGCAAGGCCACATTGA |
| | EGFR)-CL | CTGCTGACAAATCCTCCAGCACAGCCTACATGCAGCTCAGCAGCCTGACATCTGAGGACTCTGCGGT |
| | (L = G4S) | CTATTACTGTGCAAGATCGACTTACTACGGCGGTGACTGGTACTTCAATGTCTGGGGCGCAGGGACC |
| | | ACGGTCACCGTCTCCGCAgcctccaccaagggcccatcggtcttccccctggcaccctcctccaaga |
| | | gcacctctgggggcacagcggccctgggctgcctggtcaaggactacttccccgaaccggtgacggt |
| | | gtcgtggaactcaggcgccctgaccagcggcgtgcacaccttcccggctgtcctacagtcctcagga |
| | | ctctactccctcagcagcgtggtgaccgtgccctccagcagcttgggcacccagacctacatctgca |
| | | acgtgaatcacaagcccagcaacaccaaggtggacaagagagttgagcccaaatcttgtgacaaaac |
| | | tcacacatgcccaccgtgcccagcacctccagtggccggaccgtcagtcttcctcttccccccaaaa |
| | | cccaaggacaccctcatgatctcccggacccctgaggtcacatgcgtggtggtggacgtgagccacg |
| | | aagaccctgaggtcaagttcaactggtacgtggacggcgtggaggtgcataatgccaagacaaagcc |
| | | gcgggaggagcagtacaacagcacgtaccgtgtggtcagcgtcctcaccgtcctgcaccaggactgg |
| | | ctgaatggcaaggagtacaagtgcaaggtctccaacaaagccctcccatccagcatcgagaaaacca |
| | | tctccaaagccaaagggcagccccgagaaccacaggtgtacaccctgcccccatcacgggaggagat |
| | | gaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggct |
| | | ccttcttcctctatagcaagctcaccgtggacaagagcaggtggcagcaggggaacgtcttctcatg |
| | | ctccgtgatgcatgaggctctgcacaaccactacacacagaagagcctctccctgtccccgggtgca |
| | | ggtggaggtgggagcGACATCTTGCTGACTCAGTCTCCAGTCATCCTGTCTGTGAGTCCAGGAGAAA |
| | | GAGTCAGTTTCTCCTGCAGGGCCAGTCAGAGTATTGGCACAAACATACACTGGTATCAGCAAAGAAC |
| | | AAATGGTTCTCCAAGGCTTCTCATAAAGTATGCTTCTGAGTCTATCTCTGGAATCCCTTCCAGGTTT |
| | | AGTGGCAGTGGATCAGGGACAGATTTTACTCTTAGCATCAACAGTGTGGAGTCTGAAGATATTGCAG |
| | | ATTATTACTGTCAACAAAATAATAACTGGCCAACCACGTTCGGTGCTGGGACCAAGCTGGAGCTGAA |
| | | Acgaactgtggctgcaccatctgtcttcatcttcccgccatctgatgagcagttgaaatctggaact |
| | | gcctctgttgtgtgcctgctgaataacttctatcccagagaggccaaagtacagtggaaggtggata |
| | | acgccctccaatcgggtaactcccaggagagtgtcacagagcaggacagcaaggacagcacctacag |
| | | cctcagcagcaccctgacgctgagcaaagcagactacgagaaacacaaagtctacgcctgcgaagtc |
| | | acccatcagggcctgagctcgcccgtcacaaagagcttcaacaggggagagtgttga |
| 46 | VL(anti- | ATGGAGTTGCCTGTTAGGCTGTTGGTGCTGATGTTCTGGATTCCTGCTTCCTTAAGCgacattgtgc |
| | CD16)-CL | tgacccaatctccagcttctttggctgtgtctctagggcagagggccaccatctcctgcaaggccag |
| | | ccaaagtgttgattttgatggtgatagttttatgaactggtaccaacagaaaccaggacagccaccc |
| | | aaactcctcatctatactacatccaatctagaatctggcatcccagccaggtttagtgccagtgggt |
| | | ctgggacagacttcaccctcaacatccatcctgtggaggaggaggatactgcaacctattactgtca |
| | | gcaaagtaatgaggacccgtacacgttcggaggggggaccaagctggagctgaaaCGAACTGTGGCT |
| | | GCACCATCTGTCTTCATCTTCCCGCCATCTGATGAGCAGTTGAAATCTGGAACTGCCTCTGTTGTGT |
| | | GCCTGCTGAATAACTTCTATCCCAGAGAGGCCAAAGTACAGTGGAAGGTGGATAACGCCCTCCAATC |
| | | GGGTAACTCCCAGGAGAGTGTCACAGAGCAGGACAGCAAGGACAGCACCTACAGCCTCAGCAGCACC |
| | | CTGACGCTGAGCAAAGCAGACTACGAGAAACACAAAGTCTACGCCTGCGAAGTCACCCATCAGGGCC |
| | | TGAGCTCGCCCGTCACAAAGAGCTTCAACAGGGGAGAGTGTTAG |
| 47 | VH(anti- | QVTLKESGPGILQPSQTLSLTCSFSGFSLRTSGMGVGWIRQPSGKGLEWLAHIWWDDDKRYNPALKS |
| | CD16)-CH1- | RLTISKDTSSNQVFLKIASVDTADTATYYCAQINPAWFAYWGQGTLVTVSAASTKGPSVFPLAPSSK |
| | H-CH2-CH3- | STSGGTAALGCLVKDYFPEPVTVSWNSGALTSGVHTFPAVLQSSGLYSLSSVVTVPSSSLGTQTYIC |
| | L-VL(anti- | NVNHKPSNTKVDKRVEPKSCDKTHTCPPCPAPPVAGPSVFLFPPKPKDTLMISRTPEVTCVVVDVSH |
| | EGFR)-CL | EDPEVKFNWYVDGVEVHNAKTKPREEQYNSTYRVVSVLTVLHQDWLNGKEYKCKVSNKALPSSIEKT |
| | (L = G4S) | ISKAKGQPREPQVYTLPPSREEMTKNQVSLICLVKGFYPSDIAVEWESNGQPENNYKTTPPVLDSDG |
| | | SFFLYSKLTVDKSRWQQGNVFSCSVMHEALHNHYTQKSLSLSPGAGGGGSDILLTQSPVILSVSPGE |
| | | RVSFSCRASQSIGTNIHWYQQRTNGSPRLLIKYASESISGIPSRFSGSGSGTDFTLSINSVESEDIA |
| | | DYYCQQNNNWPTTFGAGTKLELKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSSPVTKSFNRGEC |
| 48 | VL(anti- | DIVLTQSPASLAVSLGQRATISCKASQSVDFDGDSFMNWYQQKPGQPPKLLIYTTSNLESGIPARFS |
| | CD16)-CL | ASGSGTDFTLNIHPVEEEDTATYYCQQSNEDPYTFGGGTKLELKRTVAAPSVFIFPPSDEQLKSGTA |
| | | SVVCLLNNFYPREAKVQWKVDNALQSGNSQESVTEQDSKDSTYSLSSTLTLSKADYEKHKVYACEVT |
| | | HQGLSSPVTKSFNRGEC |
| 49 | VH(anti- | atggagttgcctgttaggctgttggtgctgatgttctggattcctgctagctccagccaggtacaac |
| | CD20)-CH1- | tgcaacagcctggggctgagctggtgaagcctggggcctcagtgaagatgtcctgcaaggcttctgg |
| | H-CH2-CH3- | ctacacatttaccagttacaatatgcactgggtaaaacagacacctggtcggggcctggaatggatt |
| | L-VL(anti- | ggagctatttatcccggaaatggtgatacttcctacaatcagaagttcaaaggcaaggccacattga |
| | CD16)-CL | ctgctgacaaatcctccagcacagcctacatgcagctcagcagcctgacatctgaggactctgcggt |
| | (L = G4S) | ctattactgtgcaagatcgacttactacggcggtgactggtacttcaatgtctggggcgcagggacc |
| | | acggtcaccgtctccgcagcctccaccaagggcccatcggtcttccccctggcaccctcctccaaga |
| | | gcacctctgggggcacagcggccctgggctgcctggtcaaggactacttccccgaaccggtgacggt |
| | | gtcgtggaactcaggcgccctgaccagcggcgtgcacaccttcccggctgtcctacagtcctcagga |
| | | ctctactccctcagcagcgtggtgaccgtgccctccagcagcttgggcacccagacctacatctgca |
| | | acgtgaatcacaagcccagcaacaccaaggtggacaagaaagttgagcccaaatcttgtgacaaaac |
| | | tcacacatgcccaccgtgcccagcacctgaactcctggggggaccgtcagtcttcctcttcccccca |
| | | aaacccaaggacaccctcatgatctcccggacccctgaggtcacatgcgtggtggtggacgtgagcc |
| | | acgaagaccctgaggtcaagttcaactggtacgtggacggcgtggaggtgcataatgccaagacaaa |
| | | gccgcgggaggagcagtacaacagcacgtaccgtgtggtcagcgtcctcaccgtcctgcaccaggac |
| | | tggctgaatggcaaggagtacaagtgcaaggtctccaacaaagccctcccagcccccatcgagaaaa |
| | | ccatctccaaagccaaagggcagccccgagaaccacaggtgtacaccctgcccccatcacgggatga |
| | | gctgaccaagaaccaggtcagcctgacctgcctggtcaaaggcttctatcccagcgacatcgccgtg |
| | | gagtgggagagcaatgggcagccggagaacaactacaagaccacgcctcccgtgctggactccgacg |
| | | gctccttcttcctctatagcaagctcaccgtggacaagagcaggtggcagcaggggaacgtcttctc |
| | | atgctccgtgatgcatgaggctctgcacaaccactacacgcagaagagcctctccctgtccccgggt |
| | | gcaggtggaggtgggagcattgtgctgacccaatctccagcttctttggctgtgtctctagggcaga |
| | | gggccaccatctcctgcaaggccagccaaagtgttgattttgatggtgatagttttatgaactggta |
| | | ccaacagaaaccaggacagccacccaaactcctcatctatactacatccaatctagaatctgggatc |
| | | ccagccaggtttagtgccagtgggtctgggacagacttcaccctcaacatccatcctgtggaggagg |
| | | aggatactgcaacctattactgtcagcaaagtaatgaggatccgtacacgttcggaggggggaccaa |
| | | gctggagctgaaacgtggaactgtggctgcaccatctgtcttcatcttcccgccatctgatgagcag |
| | | ttgaaatctggaactgcctctgttgtgtgcctgctgaataacttctatcccagagaggccaaagtac |
| | | agtggaaggtggataacgccctccaatcgggtaactcccaggagagtgtcacagagcaggacagcaa |
| | | ggacagcacctacagcctcagcagcaccctgacgctgagcaaagcagactacgagaaacacaaagtc |
| | | tacgcctgcgaagtcacccatcagggcctgagctcgcccgtcacaaagagcttcaacaggggagagt |
| | | gttga |
| 50 | VH(anti- | ATGGAGTTGCCTGTTAGGCTGTTGGTGCTGATGTTCTGGATTCCTGCTTCCAGCAGCCAGGTTACTC |
| | CD16)-CH1 | TGAAAGAGTCTGGCCCTGGGATATTGCAGCCCTCCCAGACCCTCAGTCTGACTTGTTCTTTCTCTGG |
| | | GTTTTCACTGAGGACTTCTGGTATGGGTGTAGGCTGGATTCGTCAGCCTTCAGGGAAGGGTCTAGAG |
| | | TGGCTGGCACACATTTGGTGGGATGATGACAAGCGCTATAATCCAGCCCTGAAGAGCCGACTGACAA |
| | | TCTCCAAGGATACCTCCAGCAACCAGGTATTCCTCAAAATCGCCAGTGTGGACACTGCAGATACTGC |
| | | CACATACTACTGTGCTCAAATAAACCCCGCCTGGTTTGCTTACTGGGGCCAAGGGACTCTGGTCACT |
| | | GTCTCTGCGGCTAGCACCAAGGGCCCATCGGTCTTCCCCCTGGCACCCTCCTCCAAGAGCACCTCTG |
| | | GGGGCACAGCGGCCCTGGGCTGCCTGGTCAAGGACTACTTCCCCGAACCGGTGACGGTGTCGTGGAA |
| | | CTCAGGCGCCCTGACCAGCGGCGTGCACACCTTCCCGGCTGTCCTACAGTCCTCAGGACTCTACTCC |
| | | CTCAGCAGCGTGGTGACCGTGCCCTCCAGCAGCTTGGGCACCCAGACCTACATCTGCAACGTGAATC |
| | | ACAAGCCCAGCAACACCAAGGTGGACAAGAAAGTTTAG |
| 51 | VH(anti- | QVQLQQPGAELVKPGASVKMSCKASGYTFTSYNMHWVKQTPGRGLEWIGAIYPGNGDTSYNQKFKGK |
| | CD20)-CH1- | ATLTADKSSSTAYMQLSSLTSEDSAVYYCARSTYYGGDWYFNVWGAGTTVTVSAASTKGPSVFPLAP |
| | H-CH2-CH3- | SSKSTSGGTAALGCLVKDYFPEPVTVSWNSGALTSGVHTFPAVLQSSGLYSLSSVVTVPSSSLGTQT |
| | L-VL(anti- | YICNVNHKPSNTKVDKKVEPKSCDKTHTCPPCPAPELLGGPSVFLFPPKPKDTLMISRTPEVTCVVV |
| | CD16)-CL | DVSHEDPEVKFNWYVDGVEVHNAKTKPREEQYNSTYRVVSVLTVLHQDWLNGKEYKCKVSNKALPAP |
| | (L = G4S) | IEKTISKAKGQPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGAGGGGSIVLTQSPASLAVS |
| | | LGQRATISCKASQSVDFDGDSFMNWYQQKPGQPPKLLIYTTSNLESGIPARFSASGSGTDFTLNIHP |
| | | VEEEDTATYYCQQSNEDPYTFGGGTKLELKRGTVAAPSVFIFPPSDEQLKSGTASVVCLLNNFYPRE |
| | | AKVQWKVDNALQSGNSQESVTEQDSKDSTYSLSSTLTLSKADYEKHKVYACEVTHQGLSSPVTKSFN |
| | | RGEC |
| 52 | VH(anti- | QVTLKESGPGILQPSQTLSLTCSFSGFSLRTSGMGVGWIRQPSGKGLEWLAHIWWDDDKRYNPALKS |
| | CD16)-CH1 | RLTISKDTSSNQVFLKIASVDTADTATYYCAQINPAWFAYWGQGTLVTVSAASTKGPSVFPLAPSSK |
| | | STSGGTAALGCLVKDYFPEPVTVSWNSGALTSGVHTFPAVLQSSGLYSLSSVVTVPSSSLGTQTYIC |
| | | NVNHKPSNTKVDKKV |
| 53 | anti-CD47 | gatattgtgatgactcagtctccagccaccctgtctgtgactccaggagatagagtctctctttcct |
| | Fab light | gcagggccagccagactattagcgactacttacactggtatcaacaaaaatcacatgagtctccaag |
| | chain | gcttctcatcaaatttgcttcccaatccatttctggaatcccctccaggttcagtggcagtggatca |
| | | ggctcagatttcactctcagtatcaacagtgtggaacctgaagatgttggagtgtattactgtcaaa |
| | | atggtcacggctttcctcggacgttcggtggagggaccaagctggaaataaaacgtggaactgtggc |
| | | tgcaccatctgtcttcatcttcccgccatctgatgagcagttgaaatctggaactgcctctgttgtg |
| | | tgcctgctgaataacttctatcccagagaggccaaagtacagtggaaggtggataacgccctccaat |
| | | cgggtaactcccaggagagtgtcacagagcaggacagcaaggacagcacctacagcctcagcagcac |
| | | cctgacgctgagcaaagcagactacgagaaacacaaagtctacgcctgcgaagtcacccatcagggc |
| | | ctgagctcgcccgtcacaaagagcttcaacaggggagag |
| 54 | anti-CD47 | DIVMTQSPATLSVTPGDRVSLSCRASQTISDYLHWYQQKSHESPRLLIKFASQSISGIPSRFSGSGS |
| | Fab light | GSDFTLSINSVEPEDVGVYYCQNGHGFPRTFGGGTKLEIKRGTVAAPSVFIFPPSDEQLKSGTASVV |
| | chain | CLLNNFYPREAKVQWKVDNALQSGNSQESVTEQDSKDSTYSLSSTLTLSKADYEKHKVYACEVTHQG |
| | | LSSPVTKSFNRGEC |
| 55 | anti-CD47 | GAGGTGCAGCTGGTGGAGTCTGGGGGAGACTTAGTGAAGCCTGGAGGGTCCCTGAAACTCTCCTGTG |
| | Fab heavy | CAGCCTCTGGATTCACTTTCAGTGGCTATGGCATGTCTTGGGTTCGCCAGACTCCAGACAAGAGGCT |
| | chain | GGAGTGGGTCGCAACCATTACTAGTGGTGGTACTTACACCTACTATCCAGACAGTGTGAAGGGGCGA |
| | | TTCACCATCTCCAGAGACAATGCCAAGAACACCCTGTACCTGCAAATAGACAGTCTGAAGTCTGAGG |
| | | ATACAGCCATATATTTCTGTGCAAGATCCCTCGCGGGAAATGCTATGGACTACTGGGGTCAAGGGAC |
| | | CAGCGTCACCGTCTCCTCAGCTAGCACCAAGGGCCCATCGGTCTTCCCCCTGGCACCCTCCTCCAAG |
| | | AGCACCTCTGGGGGCACAGCGGCCCTGGGCTGCCTGGTCAAGGACTACTTCCCCGAACCGGTGACGG |
| | | TGTCGTGGAACTCAGGCGCCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGCACCCAGACCTACATCTGC |
| | | AACGTGAATCACAAGCCCAGCAACACCAAGGTGGACAAGAAAGTTGAGCCCAAATCT |
| 56 | anti-CD47 | EVQLVESGGDLVKPGGSLKLSCAASGFTFSGYGMSWVRQTPDKRLEWVATITSGGTYTYYPDSVKGR |
| | Fab heavy | FTISRDNAKNTLYLQIDSLKSEDTAIYFCARSLAGNAMDYWGQGTSVTVSSASTKGPSVFPLAPSSK |
| | chain | STSGGTAALGCLVKDYFPEPVTVSWNSGALTSGVHTFPAVLQSSGLYSLSSVVTVPSSSLGTQTYIC |
| | | NVNHKPSNTKVDKRVEPKSC |
| 57 | VH(anti- | atggagttgcctgttaggctgttggtgctgatgttctggattcctgctagctccagccaggtacaac |
| | CD20)-CH1- | tgcaacagcctggggctgagctggtgaagcctggggcctcagtgaagatgtcctgcaaggcttctgg |
| | H-CH2-CH3- | ctacacatttaccagttacaatatgcactgggtaaaacagacacctggtcggggcctggaatggatt |
| | L-VL(anti- | ggagctatttatcccggaaatggtgatacttcctacaatcagaagttcaaaggcaaggccacattga |
| | CD47)-CL | ctgctgacaaatcctccagcacagcctacatgcagctcagcagcctgacatctgaggactctgcggt |
| | (L = | ctattactgtgcaagatcgacttactacggcggtgactggtacttcaatgtctggggcgcagggacc |
| | (G4S)4) | acggtcaccgtctccgcagcctccaccaagggcccatcggtcttccccctggcaccctcctccaaga |
| | | gcacctctgggggcacagcggccctgggctgcctggtcaaggactacttccccgaaccggtgacggt |
| | | gtcgtggaactcaggcgccctgaccagcggcgtgcacaccttcccggctgtcctacagtcctcagga |
| | | ctctactccctcagcagcgtggtgaccgtgccctccagcagcttgggcacccagacctacatctgca |
| | | acgtgaatcacaagcccagcaacaccaaggtggacaagaaagttgagcccaaatcttgtgacaaaac |
| | | tcacacatgcccaccgtgcccagcacctgaactcctggggggaccgtcagtcttcctcttcccccca |
| | | aaacccaaggacaccctcatgatctcccggacccctgaggtcacatgcgtggtggtggacgtgagcc |
| | | acgaagaccctgaggtcaagttcaactggtacgtggacggcgtggaggtgcataatgccaagacaaa |
| | | gccgcgggaggagcagtacaacagcacgtaccgtgtggtcagcgtcctcaccgtcctgcaccaggac |
| | | tggctgaatggcaaggagtacaagtgcaaggtctccaacaaagccctcccagcccccatcgagaaaa |
| | | ccatctccaaagccaaagggcagccccgagaaccacaggtgtacaccctgcccccatcacgggatga |
| | | gctgaccaagaaccaggtcagcctgacctgcctggtcaaaggcttctatcccagcgacatcgccgtg |
| | | gagtgggagagcaatgggcagccggagaacaactacaagaccacgcctcccgtgctggactccgacg |
| | | gctccttcttcctctatagcaagctcaccgtggacaagagcaggtggcagcaggggaacgtcttctc |
| | | atgctccgtgatgcatgaggctctgcacaaccactacacgcagaagagcctctccctgtccccgggt |
| | | gctggcggcggaggaagcggaggaggaggcagcggaggcggaggctccggcggaggaggctccggag |
| | | atattgtgatgactcagtctccagccaccctgtctgtgactccaggagatagagtctctctttcctg |
| | | cagggccagccagactattagcgactacttacactggtatcaacaaaaatcacatgagtctccaagg |
| | | cttctcatcaaatttgcttcccaatccatttctggaatcccctccaggttcagtggcagtggatcag |
| | | gctcagatttcactctcagtatcaacagtgtggaacctgaagatgttggagtgtattactgtcaaaa |
| | | tggtcacggctttcctcggacgttcggtggagggaccaagctggaaataaaacgtggaactgtggct |
| | | gcaccatctgtcttcatcttcccgccatctgatgagcagttgaaatctggaactgcctctgttgtgt |
| | | gcctgctgaataacttctatcccagagaggccaaagtacagtggaaggtggataacgccctccaatc |
| | | gggtaactcccaggagagtgtcacagagcaggacagcaaggacagcacctacagcctcagcagcacc |
| | | ctgacgctgagcaaagcagactacgagaaacacaaagtctacgcctgcgaagtcacccatcagggcc |
| | | tgagctcgcccgtcacaaagagcttcaacaggggagagtgttga |
| 58 | VH(anti- | ATGGAGTTGCCTGTTAGGCTGTTGGTGCTGATGTTCTGGATTCCTGCTTCCAGCAGCGAGGTGCAGC |
| | CD47)-CH1- | TGGTGGAGTCTGGGGGAGACTTAGTGAAGCCTGGAGGGTCCCTGAAACTCTCCTGTGCAGCCTCTGG |
| | H* | ATTCACTTTCAGTGGCTATGGCATGTCTTGGGTTCGCCAGACTCCAGACAAGAGGCTGGAGTGGGTC |
| | | GCAACCATTACTAGTGGTGGTACTTACACCTACTATCCAGACAGTGTGAAGGGGCGATTCACCATCT |
| | | CCAGAGACAATGCCAAGAACACCCTGTACCTGCAAATAGACAGTCTGAAGTCTGAGGATACAGCCAT |
| | | ATATTTCTGTGCAAGATCCCTCGCGGGAAATGCTATGGACTACTGGGGTCAAGGGACCAGCGTCACC |
| | | GTCTCCTCAGCTAGCACCAAGGGCCCATCGGTCTTCCCCCTGGCACCCTCCTCCAAGAGCACCTCTG |
| | | GGGGCACACGGCCCTGGGCTGCCTGGTCAAGGACTACTTCCCCGAACCGGTGACGGTGTCGTGGAAC |
| | | TCAGGCGCCCTGACCAGCGGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCTACATCTGCAACGTGAATCA |
| | | CAAGCCCAGCAACACCAAGGTGGACAAGAAAGTTGAGCCCAAATCTTGTTAG |
| 59 | VH(anti- | QVQLQQPGAELVKPGASVKMSCKASGYTFTSYNMHWVKQTPGRGLEWIGAIYPGNGDTSYNQKFKGK |
| | CD20)-CH1- | ATLTADKSSSTAYMQLSSLTSEDSAVYYCARSTYYGGDWYFNVWGAGTTVTVSAASTKGPSVFPLAP |
| | H-CH2-CH3- | SSKSTSGGTAALGCLVKDYFPEPVTVSWNSGALTSGVHTFPAVLQSSGLYSLSSVVTVPSSSLGTQT |
| | L-VL(anti- | YICNVNHKPSNTKVDKKVEPKSCDKTHTCPPCPAPELLGGPSVFLFPPKPKDTLMISRTPEVTCVVV |
| | CD47)-CL | DVSHEDPEVKFNWYVDGVEVHNAKTKPREEQYNSTYRVVSVLTVLHQDWLNGKEYKCKVSNKALPAP |
| | (L = | IEKTISKAKGQPREPQVYTLPPSRDELTKNQVSLTCLVKGFYPSDIAVEWESNGQPENNYKTTPPVL |
| | (G4S)4) | DSDGSFFLYSKLTVDKSRWQQGNVFSCSVMHEALHNHYTQKSLSLSPGAGGGGSGGGGSGGGGSGGG |
| | | GSGDIVMTQSPATLSVTPGDRVSLSCRASQTISDYLHWYQQKSHESPRLLIKFASQSISGIPSRFSG |
| | | SGSGSDFTLSINSVEPEDVGVYYCQNGHGFPRTFGGGTKLEIKRGTVAAPSVFIFPPSDEQLKSGTA |
| | | SVVCLLNNFYPREAKVQWKVDNALQSGNSQESVTEQDSKDSTYSLSSTLTLSKADYEKHKVYACEVT |
| | | HQGLSSPVTKSFNRGEC |
| 60 | VH(anti- | EVQLVESGGDLVKPGGSLKLSCAASGFTFSGYGMSWVRQTPDKRLEWVATITSGGTYTYYPDSVKGR |
| | CD47)-CH1- | FTISRDNAKNTLYLQIDSLKSEDTAIYFCARSLAGNAMDYWGQGTSVTVSSASTKGPSVFPLAPSSK |
| | H* | STSGGTAALGCLVKDYFPEPVTVSWNSGALTSGVHTFPAVLQSSGLYSLSSVVTVPSSSLGTQTYIC |
| | | NVNHKPSNTKVDKRVEPKSC |
| 61 | anti-CD52 | gacatccagatgacccagagcccaagcagcctgagcgccagcgtgggtgacagagtgaccatcacct |
| | Fab light | gtaaagcaagtcagaatattgacaaatacttaaactggtaccagcagaagccaggtaaggctccaaa |
| | chain | gctgctgatctacaatacaaacaatttgcaaacgggtgtgccaagcagattcagcggtagcggtagc |
| | | ggtaccgacttcaccttcaccatcagcagcctccagccagaggacatcgccacctactactgcttgc |
| | | agcatataagtaggccgcgcacgttcggccaagggaccaaggtggaaatcaaacgtggaactgtggc |
| | | tgcaccatctgtcttcatcttcccgccatctgatgagcagttgaaatctggaactgcctctgttgtg |
| | | tgcctgctgaataacttctatcccagagaggccaaagtacagtggaaggtggataacgccctccaat |
| | | cgggtaactcccaggagagtgtcacagagcaggacagcaaggacagcacctacagcctcagcagcac |
| | | cctgacgctgagcaaagcagactacgagaaacacaaagtctacgcctgcgaagtcacccatcagggc |
| | | ctgagctcgcccgtcacaaagagcttcaacaggggagag |
| 62 | anti-CD52 | QIVLSQSPAILSASPGEKVTMTCRASSSVSYIHWFQQKPGSSPKPWIYATSNLASGVPVRFSGSGSG |
| | Fab light | TSYSLTISRVEAEDAATYYCQQWTSNPPTFGGGTKLEIKGTVAAPSVFIFPPSDEQLKSGTASVVCL |
| | chain | LNNFYPREAKVQWKVDNALQSGNSQESVTEQDSKDSTYSLSSTLTLSKADYEKHKVYACEVTHQGLS |
| | | SPVTKSFNRGEC |
| 63 | anti-CD52 | CAGGTACAACTGCAACAGCCTGGGGCTGAGCTGGTGAAGCCTGGGGCCTCAGTGAAGATGTCCTGCA |
| | Fab heavy | AGGCTTCTGGCTACACATTTACCAGTTACAATATGCACTGGGTAAAACAGACACCTGGTCGGGGCCT |
| | chain | GGAATGGATTGGAGCTATTTATCCCGGAAATGGTGATACTTCCTACAATCAGAAGTTCAAAGGCAAG |
| | | GCCACATTGACTGCTGACAAATCCTCCAGCACAGCCTACATGCAGCTCAGCAGCCTGACATCTGAGG |
| | | ACTCTGCGGTCTATTACTGTGCAAGATCGACTTACTACGGCGGTGACTGGTACTTCAATGTCTGGGG |
| | | CGCAGGGACCACGGTCACCGTCTCCGCAGCTAGCACCAAGGGCCCATCGGTCTTCCCCCTGGCACCC |
| | | TCCTCCAAGAGCACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTGCACACCTTCCCGGCTGTCCTACA |
| | | GTCCTCAGGACTCTACTCCCTCAGCAGCGTGGTGACCGTGCCCTCCAGCAGCTTGGGCACCCAGACC |
| | | TACATCTGCAACGTGAATCACAAGCCCAGCAACACCAAGGTGGACAAGAAAGTT |
| 64 | anti-CD52 | QVQLQESGPGLVRPSQTLSLTCTVSGFTFTDFYMNWVRQPPGRGLEWIGFIRDKAKGYTTEYNPSVK |
| | Fab heavy | GRVTMLVDTSKNQFSLRLSSVTAADTAVYYCAREGHTAAPFDYWGQGSLVTVSAASTKGPSVFPLAP |
| | chain | SSKSTSGGTAALGCLVKDYFPEPVTVSWNSGALTSGVHTFPAVLQSSGLYSLSSVVTVPSSSLGTQT |
| | | YICNVNHKPSNTKVDKKV |
| 65 | VH(anti- | atggagttgcctgttaggctgttggtgctgatgttctggattcctgctagctccagccaggtacaac |
| | CD20)-CH1- | tgcaacagcctggggctgagctggtgaagcctggggcctcagtgaagatgtcctgcaaggcttctgg |
| | H-CH2-CH3- | ctacacatttaccagttacaatatgcactgggtaaaacagacacctggtcggggcctggaatggatt |
| | L-VL(anti- | ggagctatttatcccggaaatggtgatacttcctacaatcagaagttcaaaggcaaggccacattga |
| | CD52)-CL | ctgctgacaaatcctccagcacagcctacatgcagctcagcagcctgacatctgaggactctgcggt |
| | (L = G4S) | ctattactgtgcaagatcgacttactacggcggtgactggtacttcaatgtctggggcgcagggacc |
| | | acggtcaccgtctccgcagcctccaccaagggcccatcggtcttccccctggcaccctcctccaaga |
| | | gcacctctgggggcacagcggccctgggctgcctggtcaaggactacttccccgaaccggtgacggt |
| | | gtcgtggaactcaggcgccctgaccagcggcgtgcacaccttcccggctgtcctacagtcctcagga |
| | | ctctactccctcagcagcgtggtgaccgtgccctccagcagcttgggcacccagacctacatctgca |
| | | acgtgaatcacaagcccagcaacaccaaggtggacaagaaagttgagcccaaatcttgtgacaaaac |
| | | tcacacatgcccaccgtgcccagcacctgaactcctggggggaccgtcagtcttcctcttcccccca |
| | | aaacccaaggacaccctcatgatctcccggacccctgaggtcacatgcgtggtggtggacgtgagcc |
| | | acgaagaccctgaggtcaagttcaactggtacgtggacggcgtggaggtgcataatgccaagacaaa |
| | | gccgcgggaggagcagtacaacagcacgtaccgtgtggtcagcgtcctcaccgtcctgcaccaggac |
| | | tggctgaatggcaaggagtacaagtgcaaggtctccaacaaagccctcccagcccccatcgagaaaa |
| | | ccatctccaaagccaaagggcagccccgagaaccacaggtgtacaccctgcccccatcacgggatga |
| | | gctgaccaagaaccaggtcagcctgacctgcctggtcaaaggcttctatcccagcgacatcgccgtg |
| | | gagtgggagagcaatgggcagccggagaacaactacaagaccacgcctcccgtgctggactccgacg |
| | | gctccttcttcctctatagcaagctcaccgtggacaagagcaggtggcagcaggggaacgtcttctc |
| | | atgctccgtgatgcatgaggctctgcacaaccactacacgcagaagagcctctccctgtccccgggt |
| | | gcaggtggaggtgggagcgacatccagatgacccagagcccaagcagcctgagcgccagcgtgggtg |
| | | acagagtgaccatcacctgtaaagcaagtcagaatattgacaaatacttaaactggtaccagcagaa |
| | | gccaggtgaggctccaaagctgctgatctacaatacaaacaatttgcaaacgggtgtgccaagcaga |
| | | ttcagcggtagcggtagcggtaccgacttcaccttcaccatcagcagcctccagccagaggacatcg |
| | | ccacctactactgcttgcagcatataagtaggccgcgcacgttcggccaagggaccaaggtggaaat |
| | | caaacgtggaactgtggctgcaccatctgtcttcatcttcccgccatctgatgagcagttgaaatct |
| | | ggaactgcctctgttgtgtgcctgctgaataacttctatcccagagaggccaaagtacagtggaagg |
| | | tggataacgccctccaatcgggtaactcccaggagagtgtcacagagcaggacagcaaggacagcac |
| | | ctacagcctcagcagcaccctgacgctgagcaaagcagactacgagaaacacaaagtctacgcctgc |
| | | gaagtcacccatcagggcctgagctcgcccgtcacaaagagcttcaacaggggagagtgttga |
| 66 | VH(anti- | ATGGAGTTGCCTGTTAGGCTGTTGGTGCTGATGTTCTGGATTCCTGCTTCCAGCAGCCAGGTACAAC |
| | CD52)-CH1- | TGCAACAGCCTGGGGCTGAGCTGGTGAAGCCTGGGGCCTCAGTGAAGATGTCCTGCAAGGCTTCTGG |
| | H* | CTACACATTTACCAGTTACAATATGCACTGGGTAAAACAGACACCTGGTCGGGGCCTGGAATGGATT |
| | | GGAGCTATTTATCCCGGAAATGGTGATACTTCCTACAATCAGAAGTTCAAAGGCAAGGCCACATTGA |
| | | CTGCTGACAAATCCTCCAGCACAGCCTACATGCAGCTCAGCAGCCTGACATCTGAGGACTCTGCGGT |
| | | CTATTACTGTGCAAGATCGACTTACTACGGCGGTGACTGGTACTTCAATGTCTGGGGCGCAGGGACC |
| | | ACGGTCACCGTCTCCGCAGCTAGCACCAAGGGCCCATCGGTCTTCCCCCTGGCACCCTCCTCCAAGA |
| | | GCACCTCTGGGGGCACAGCGGCCCTGGGCTGCCTGGTCAAGGACTACTTCCCCGAACCGGTGACGGT |
| | | GTCGTGGAACTCAGGCGCCCTGACCAGCGGCGTGCACACCTTCCCGGCTGTCCTACAGTCCTCAGGA |
| | | CTCTACTCCCTCAGCAGCGTGGTGACCGTGCCCTCCAGCAGCTTGGGCACCCAGACCTACATCTGCG |
| | | CCGTGATCAGCAGCGGCGGCAGCTCCATCAACTACAAGAAAGTTGAGCCCAAATCTTGTTAA |
| 67 | VH(anti- | QVQLQQPGAELVKPGASVKMSCKASGYTFTSYNMHWVKQTPGRGLEWIGAIYPGNGDTSYNQKFKGK |
| | CD20)-CH1- | ATLTADKSSSTAYMQLSSLTSEDSAVYYCARSTYYGGDWYFNVWGAGTTVTVSAASTKGPSVFPLAP |
| | H-CH2-CH3- | SSKSTSGGTAALGCLVKDYFPEPVTVSWNSGALTSGVHTFPAVLQSSGLYSLSSVVTVPSSSLGTQT |
| | L-VL(anti- | YICNVNHKPSNTKVDKKVEPKSCDKTHTCPPCPAPELLGGPSVFLFPPKPKDTLMISRTPEVTCVVV |
| | CD52)-CL | DVSHEDPEVKFNWYVDGVEVHNAKTKPREEQYNSTYRVVSVLTVLHQDWLNGKEYKCKVSNKALPAP |
| | (L = G4S) | IEKTISKAKGQPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGAGGGGSDIQMTQSPSSLSA |
| | | SVGDRVTITCKASQNIDKYLNWYQQKPGKAPKLLIYNTNNLQTGVPSRFSGSGSGTDFTFTISSLQP |
| | | EDIATYYCLQHISRPRTFGQGTKVEIKGTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGLSSPVTKSFNRGEC |
| 68 | VH1(anti- | QVQLQESGPGLVRPSQTLSLTCTVSGFTFTDFYMNWVRQPPGRGLEWIGFIRDKAKGYTTEYNPSVK |
| | CD52)-CH1- | GRVTMLVDTSKNQFSLRLSSVTAADTAVYYCAREGHTAAPFDYWGQGSLVTVSAASTKGPSVFPLAP |
| | H* | SSKSTSGGTAALGCLVKDYFPEPVTVSWNSGALTSGVHTFPAVLQSSGLYSLSSVVTVPSSSLGTQT |
| | | YICNVNHKPSNTKVDKKVEPKSC |
| 69 | VH(anti- | atggagttgcctgttaggctgttggtgctgatgttctggattcctgctagctccagccaggtccaac |
| | CD52)-CH1- | tgcaggagagcggtccaggtcttgtgagacctagccagaccctgagcctgacctgcaccgtgtctgg |
| | H-CH2-CH3- | cttcaccttcaccgatttctacatgaactgggtgagacagccacctggacgaggtcttgagtggatt |
| | L-VL(anti- | ggatttattagagacaaagctaaaggttacacaacagagtacaatccatctgtgaaggggagagtga |
| | CD20)-CL | caatgctggtagacaccagcaagaaccagttcagcctgagactcagcagcgtgacagccgccgacac |
| | (L = G4S) | cgcggtctattattgtgcaagagagggccacactgctgctccttttgattactggggtcaaggcagc |
| | | ctcgtcacagtctcctcagcctccaccaagggcccatcggtcttccccctggcaccctcctccaaga |
| | | gcacctctgggggcacagcggccctgggctgcctggtcaaggactacttccccgaaccggtgacggt |
| | | gtcgtggaactcaggcgccctgaccagcggcgtgcacaccttcccggctgtcctacagtcctcagga |
| | | ctctactccctcagcagcgtggtgaccgtgccctccagcagcttgggcacccagacctacatctgca |
| | | acgtgaatcacaagcccagcaacaccaaggtggacaagaaagttgagcccaaatcttgtgacaaaac |
| | | tcacacatgcccaccgtgcccagcacctgaactcctggggggaccgtcagtcttcctcttcccccca |
| | | aaacccaaggacaccctcatgatctcccggacccctgaggtcacatgcgtggtggtggacgtgagcc |
| | | acgaagaccctgaggtcaagttcaactggtacgtggacggcgtggaggtgcataatgccaagacaaa |
| | | gccgcgggaggagcagtacaacagcacgtaccgtgtggtcagcgtcctcaccgtcctgcaccaggac |
| | | tggctgaatggcaaggagtacaagtgcaaggtctccaacaaagccctcccagcccccatcgagaaaa |
| | | ccatctccaaagccaaagggcagccccgagaaccacaggtgtacaccctgcccccatcacgggatga |
| | | gctgaccaagaaccaggtcagcctgacctgcctggtcaaaggcttctatcccagcgacatcgccgtg |
| | | gagtgggagagcaatgggcagccggagaacaactacaagaccacgcctcccgtgctggactccgacg |
| | | gctccttcttcctctatagcaagctcaccgtggacaagagcaggtggcagcaggggaacgtcttctc |
| | | atgctccgtgatgcatgaggctctgcacaaccactacacgcagaagagcctctccctgtccccgggt |
| | | gcaggtggaggtgggagccaaattgttctctcccagtctccagcaatcctgtctgcatctccagggg |
| | | agaaggtcacaatgacttgcagggccagctcaagtgtaagttacatccactggttccagcagaagcc |
| | | aggttcctcccccaaaccctggatttatgccacatccaacctggcttctggagtccctgttcgcttc |
| | | agtggcagtgggtctgggacttcttactctctcaccatcagcagagtggaggctgaagatgctgcca |
| | | cttattactgccagcagtggactagtaacccacccacgttcggaggggggaccaagctggaaatcaa |
| | | acgtggaactgtggctgcaccatctgtcttcatcttcccgccatctgatgagcagttgaaatctgga |
| | | actgcctctgttgtgtgcctgctgaataacttctatcccagagaggccaaagtacagtggaaggtgg |
| | | ataacgccctccaatcgggtaactcccaggagagtgtcacagagcaggacagcaaggacagcaccta |
| | | cagcctcagcagcaccctgacgctgagcaaagcagactacgagaaacacaaagtctacgcctgcgaa |
| | | gtcacccatcagggcctgagctcgcccgtcacaaagagcttcaacaggggagagtgttga |
| 70 | VL(anti- | ATGGAGTTGCCTGTTAGGCTGTTGGTGCTGATGTTCTGGATTCCTGCTTCCTTAAGCGACATCCAGA |
| | CD52)-CL | TGACCCAGAGCCCAAGCAGCCTGAGCGCCAGCGTGGGTGACAGAGTGACCATCACCTGTAAAGCAAG |
| | | TCAGAATATTGACAAATACTTAAACTGGTACCAGCAGAAGCCAGGTAAGGCTCCAAAGCTGCTGATC |
| | | TACAATACAAACAATTTGCAAACGGGTGTGCCAAGCAGATTCAGCGGTAGCGGTAGCGGTACCGACT |
| | | TCACCTTCACCATCAGCAGCCTCCAGCCAGAGGACATCGCCACCTACTACTGCTTGCAGCATATAAG |
| | | TAGGCCGCGCACGTTCGGCCAAGGGACCAAGGTGGAAATCAAAACTGTGGCTGCACCATCTGTCTTC |
| | | ATCTTCCCGCCATCTGATGAGCAGTTGAAATCTGGAACTGCCTCTGTTGTGTGCCTGCTGAATAACT |
| | | TCTATCCCAGAGAGGCCAAAGTACAGTGGAAGGTGGATAACGCCCTCCAATCGGGTAACTCCCAGGA |
| | | GAGTGTCACAGAGCAGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCTGAGCTCGCCCGTCA |
| | | CAAAGAGCTTCAACAGGGGAGAGTGT |
| 71 | VH(anti- | QVQLQESGPGLVRPSQTLSLTCTVSGFTFTDFYMNWVRQPPGRGLEWIGFIRDKAKGYTTEYNPSVK |
| | CD52)-CH1- | GRVTMLVDTSKNQFSLRLSSVTAADTAVYYCAREGHTAAPFDYWGQGSLVTVSSASTKGPSVFPLAP |
| | H-CH2-CH3- | SSKSTSGGTAALGCLVKDYFPEPVTVSWNSGALTSGVHTFPAVLQSSGLYSLSSVVTVPSSSLGTQT |
| | L-VL(anti- | YICNVNHKPSNTKVDKKVEPKSCDKTHTCPPCPAPELLGGPSVFLFPPKPKDTLMISRTPEVTCVVV |
| | CD20)-CL | DVSHEDPEVKFNWYVDGVEVHNAKTKPREEQYNSTYRVVSVLTVLHQDWLNGKEYKCKVSNKALPAP |
| | (L = G4S) | IEKTISKAKGQPREPQVYTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGAGGGGSQIVLSQSPAILSA |
| | | SPGEKVTMTCRASSSVSYIHWFQQKPGSSPKPWIYATSNLASGVPVRFSGSGSGTSYSLTISRVEAE |
| | | DAATYYCQQWTSNPPTFGGGTKLEIKGTVAAPSVFIFPPSDEQLKSGTASVVCLLNNFYPREAKVQW |
| | | KVDNALQSGNSQESVTEQDSKDSTYSLSSTLTLSKADYEKHKVYACEVTHQGLSSPVTKSFNRGEC |
| 72 | VL(anti- | DIQMTQSPSSLSASVGDRVTITCKASQNIDKYLNWYQQKPGKAPKLLIYNTNNLQTGVPSRFSGSGS |
| | CD52)-CL | GTDFTFTISSLQPEDIATYYCLQHISRPRTFGQGTKVEIKGTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 73 | H-CH2-CH3- | atgaagcttcctgttaggctgttggtgctgatgttctggatccctgctagcttaagcgagcccaaat |
| | L-VH(anti- | cttctgacaaaactcacacatgcccaccgtgcccagcacctgaactcctgggggggccctcagtctt |
| | CD47)-CH1- | cctcttccccccaaaacccaaggacaccctcatgatctctagaacccctgaggtcacatgcgtggtg |
| | H* (L = | gtggacgtgagccacgaagaccctgaggtcaagttcaactggtacgtggacggcgtggaggtgcata |
| | (G4S)4) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggcggcggaggaagcggaggaggtggcagcggtggcggtggctccggcgga |
| | | ggtggctccggaGAGGTGCAGCTGGTGGAGTCTGGGGGAGACTTAGTGAAGCCTGGAGGGTCCCTGA |
| | | AACTCTCCTGTGCAGCCTCTGGATTCACTTTCAGTGGCTATGGCATGTCTTGGGTTCGCCAGACTCC |
| | | AGACAAGAGGCTGGAGTGGGTCGCAACCATTACTAGTGGTGGTACTTACACCTACTATCCAGACAGT |
| | | GTGAAGGGGCGATTCACCATCTCCAGAGACAATGCCAAGAACACCCTGTACCTGCAAATAGACAGTC |
| | | TGAAGTCTGAGGATACAGCCATATATTTCTGTGCAAGATCCCTCGCGGGAAATGCTATGGACTACTG |
| | | GGGTCAAGGGACCAGCGTCACCGTCTCCTCAGCCTCCACCAAGGGCCCATCGGTCTTCCCCCTGGCA |
| | | CCCTCCTCCAAGAGCACCTCTGGGGGCACAGCGGCCCTGGGCTGCCTGGTCAAGGACTACTTCCCCG |
| | | AACCGGTGACGGTGTCGTGGAACTCAGGCGCCCTGACCAGCGGCGTGCACACCTTCCCGGCTGTCCT |
| | | ACAGTCCTCAGGACTCTACTCCCTCAGCAGCGTGGTGACCGTGCCCTCCAGCAGCTTGGGCACCCAG |
| | | ACCTACATCTGCAACGTGAATCACAAGCCCAGCAACACCAAGGTGGACAAGAAAGTTGAGCCCAAAT |
| | | CTTGTTGA |
| 74 | VL(anti- | ATGGAGTTGCCTGTTAGGCTGTTGGTGCTGATGTTCTGGATTCCTGCTTCCTTAAGCgatattgtga |
| | CD47)-CL | tgactcagtctccagccaccctgtctgtgactccaggagatagagtctctctttcctgcagggccag |
| | | ccagactattagcgactacttacactggtatcaacaaaaatcacatgagtctccaaggcttctcatc |
| | | aaatttgcttcccaatccatttctggaatcccctccaggttcagtggcagtggatcaggctcagatt |
| | | tcactctcagtatcaacagtgtggaacctgaagatgttggagtgtattactgtcaaaatggtcacgg |
| | | ctttcctcggacgttcggtggagggaccaagctggaaataaaaCGAACTGTGGCTGCACCATCTGTC |
| | | TTCATCTTCCCGCCATCTGATGAGCAGTTGAAATCTGGAACTGCCTCTGTTGTGTGCCTGCTGAATA |
| | | ACTTCTATCCCAGAGAGGCCAAAGTACAGTGGAAGGTGGATAACGCCCTCCAATCGGGTAACTCCCA |
| | | GGAGAGTGTCACAGAGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGCCTGAGCTCGCCCG |
| | | TCACAAAGAGCTTCAACAGGGGAGAGTGTTAG |
| 75 | H-CH2-CH3- | EPKSSDKTHTCPPCPAPELLGGPSVFLFPPKPKDTLMISRTPEVTCVVVDVSHEDPEVKFNWYVDGV |
| | L-VH(anti- | EVHNAKTKPREEQYNSTYRVVSVLTVLHQDWLNGKEYKCKVSNKALPAPIEKTISKAKGQPREPQVY |
| | CD47)-CH1- | TLPPSRDELTKNQVSLTCLVKGFYPSDIAVEWESNGQPENNYKTTPPVLDSDGSFFLYSKLTVDKSR |
| | H* (L = | WQQGNVFSCSVMHEALHNHYTQKSLSLSPGAGGGGSGGGGSGGGGSGGGGSGEVQLVESGGDLVKPG |
| | (G4S)4) | GSLKLSCAASGFTFSGYGMSWVRQTPDKRLEWVATITSGGTYTYYPDSVKGRFTISRDNAKNTLYLQ |
| | | IDSLKSEDTAIYFCARSLAGNAMDYWGQGTSVTVSSASTKGPSVFPLAPSSKSTSGGTAALGCLVKD |
| | | YFPEPVTVSWNSGALTSGVHTFPAVLQSSGLYSLSSVVTVPSSSLGTQTYICNVNHKPSNTKVDKKV |
| | | EPKSC |
| 76 | VL(anti- | DIVMTQSPATLSVTPGDRVSLSCRASQTISDYLHWYQQKSHESPRLLIKFASQSISGIPSRFSGSGS |
| | CD47)-CL | GSDFTLSINSVEPEDVGVYYCQNGHGFPRTFGGGTKLEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 77 | H-CH2-CH3- | atgaagcttcctgttaggctgttggtgctgatgttctggatccctgctagcttaagcgagcccaaat |
| | L-VL(anti- | cttctgacaaaactcacacatgcccaccgtgcccagcacctgaactcctgggggggccctcagtctt |
| | CD47)-CL | cctcttccccccaaaacccaaggacaccctcatgatctctagaacccctgaggtcacatgcgtggtg |
| | (L = | gtggacgtgagccacgaagaccctgaggtcaagttcaactggtacgtggacggcgtggaggtgcata |
| | (G4S)4) | atgccaagacaaagccgcgggaggagcagtacaacagcacgtaccgtgtggtcagcgtcctcaccgt |
| | | cctgcaccaggactggctgaatggcaaggagtacaagtgcaaggtctccaacaaagccctcccagcc |
| | | cccatcgagaaaacgatatccaaagccaaagggcagccccgagaaccacaggtgtacaccctgcccc |
| | | catcacgggaggagatgaccaagaaccaggtcagcctgacctgcctggtcaaaggcttctatcccag |
| | | cgacatcgccgtggagtgggagagcaatgggcagccggagaacaactacaagaccacgcctcccgtg |
| | | ctggactccgacggctccttcttcctctatagcaagctcaccgtggacaagagcaggtggcagcagg |
| | | ggaacgtcttctcatgctccgtgatgcatgaggctctgcacaaccactacacgcagaagagcgccac |
| | | cgcgaccccgggtgcaggcggcggaggaagcggaggaggtggcagcggtggcggtggctccggcgga |
| | | ggtggctccggagatattgtgatgactcagtctccagccaccctgtctgtgactccaggagatagag |
| | | tctctctttcctgcagggccagccagactattagcgactacttacactggtatcaacaaaaatcaca |
| | | tgagtctccaaggcttctcatcaaatttgcttcccaatccatttctggaatcccctccaggttcagt |
| | | ggcagtggatcaggctcagatttcactctcagtatcaacagtgtggaacctgaagatgttggagtgt |
| | | attactgtcaaaatggtcacggctttcctcggacgttcggtggagggaccaagctggaaataaaaCG |
| | | AACTGTGGCTGCACCATCTGTCTTCATCTTCCCGCCATCTGATGAGCAGTTGAAATCTGGAACTGCC |
| | | TCTGTTGTGTGCCTGCTGAATAACTTCTATCCCAGAGAGGCCAAAGTACAGTGGAAGGTGGATAACG |
| | | CCCTCCAATCGGGTAACTCCCAGGAGAGTGTCACAGAGCAGGACAGCAAGGACAGCACCTACAGCCT |
| | | CAGCAGCACCCTGACGCTGAGCAAAGCAGACTACGAGAAACACAAAGTCTACGCCTGCGAAGTCACC |
| | | CATCAGGGCCTGAGCTCGCCCGTCACAAAGAGCTTCAACAGGGGAGAGTGTTAG |
| 78 | H-CH2-CH3- | EPKSSDKTHTCPPCPAPPVAGPSVFLFPPKPKDTLMISRTPEVTCVVVDVSHEDPEVKFNWYVDGVE |
| | L-VL(anti- | VHNAKTKPREEQYNSTYRVVSVLTVLHQDWLNGKEYKCKVSNKALPSSIEKTISKAKGQPREPQVYT |
| | CD47)-CL | LPPSREEMTKNQVSLTCLVKGFYPSDIAVEWESNGQPENNYKTTPPVLDSDGSFFLYSKLTVDKSRW |
| | (L = | QQGNVFSCSVMHEALHNHYTQKSLSLSPGAGGGGSGGGGSGGGGSGGGGSGDIVMTQSPATLSVTPG |
| | (G4S)4) | DRVSLSCRASQTISDYLHWYQQKSHESPRLLIKFASQSISGIPSRFSGSGSGSDFTLSINSVEPEDV |
| | | GVYYCQNGHGFPRTFGGGTKLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQGLSSPVTKSFNRGEC |
| 79 | VH(anti- | atggagttgcctgttaggctgttggtgctgatgttctggattcctgctagctccagccaggtgcagc |
| | EGFR)-CH1- | tgaagcagtcaggacctggcctagtgcagccctcacagagcctgtccatcacctgcacagtctctgg |
| | H-CH2-CH3- | tttctcattaactaactatggtgtacactgggttcgccagtctccaggaaagggtctggagtggctg |
| | L-VL(anti- | ggagtgatatggagtggtggaaacacagactataatacacctttcacatccagactgagcatcaaca |
| | CD47)-CL | aggacaattccaagagccaagttttctttaaaatgaacagtctgcaatctaatgacacagccatata |
| | (L = | ttactgtgccagagccctcacctactatgattacgagtttgcttactggggccaagggactctggtc |
| | (G4S)4) | actgtctctgcagcctccaccaagggcccatcggtcttccccctggcaccctcctccaagagcacct |
| | | ctgggggcacagcggccctgggctgcctggtcaaggactacttccccgaaccggtgacggtgtcgtg |
| | | gaactcaggcgccctgaccagcggcgtgcacaccttcccggctgtcctacagtcctcaggactctac |
| | | tccctcagcagcgtggtgaccgtgccctccagcagcttgggcacccagacctacatctgcaacgtga |
| | | atcacaagcccagcaacaccaaggtggacaagaaagttgagcccaaatcttgtgacaaaactcacac |
| | | atgcccaccgtgcccagcacctgaactcctggggggaccgtcagtcttcctcttccccccaaaaccc |
| | | aaggacaccctcatgatctcccggacccctgaggtcacatgcgtggtggtggacgtgagccacgaag |
| | | accctgaggtcaagttcaactggtacgtggacggcgtggaggtgcataatgccaagacaaagccgcg |
| | | ggaggagcagtacaacagcacgtaccgtgtggtcagcgtcctcaccgtcctgcaccaggactggctg |
| | | aatggcaaggagtacaagtgcaaggtctccaacaaagccctcccagcccccatcgagaaaaccatct |
| | | ccaaagccaaagggcagccccgagaaccacaggtgtacaccctgcccccatcacgggatgagctgac |
| | | caagaaccaggtcagcctgacctgcctggtcaaaggcttctatcccagcgacatcgccgtggagtgg |
| | | gagagcaatgggcagccggagaacaactacaagaccacgcctcccgtgctggactccgacggctcct |
| | | tcttcctctatagcaagctcaccgtggacaagagcaggtggcagcaggggaacgtcttctcatgctc |
| | | cgtgatgcatgaggctctgcacaaccactacacgcagaagagcctctccctgtccccgggtgctggc |
| | | ggcggaggaagcggaggaggaggcagcggaggcggaggctccggcggaggaggctccggagatattg |
| | | tgatgactcagtctccagccaccctgtctgtgactccaggagatagagtctctctttcctgcagggc |
| | | cagccagactattagcgactacttacactggtatcaacaaaaatcacatgagtctccaaggcttctc |
| | | atcaaatttgcttcccaatccatttctggaatcccctccaggttcagtggcagtggatcaggctcag |
| | | atttcactctcagtatcaacagtgtggaacctgaagatgttggagtgtattactgtcaaaatggtca |
| | | cggctttcctcggacgttcggtggagggaccaagctggaaataaaacgtggaactgtggctgcacca |
| | | tctgtcttcatcttcccgccatctgatgagcagttgaaatctggaactgcctctgttgtgtgcctgc |
| | | tgaataacttctatcccagagaggccaaagtacagtggaaggtggataacgccctccaatcgggtaa |
| | | ctcccaggagagtgtcacagagcaggacagcaaggacagcacctacagcctcagcagcaccctgacg |
| | | ctgagcaaagcagactacgagaaacacaaagtctacgcctgcgaagtcacccatcagggcctgagct |
| | | cgcccgtcacaaagagcttcaacaggggagagtgttga |
| 80 | VH(anti- | QVQLKQSGPGLVQPSQSLSITCTVSGFSLTNYGVHWVRQSPGKGLEWLGVIWSGGNTDYNTPFTSRL |
| | EGFR)-CH1- | SINKDNSKSQVFFKMNSLQSNDTAIYYCARALTYYDYEFAYWGQGTLVTVSAASTKGPSVFPLAPSS |
| | H-CH2-CH3- | KSTSGGTAALGCLVKDYFPEPVTVSWNSGALTSGVHTFPAVLQSSGLYSLSSVVTVPSSSLGTQTYI |
| | L-VL(anti- | CNVNHKPSNTKVDKKVEPKSCDKTHTCPPCPAPELLGGPSVFLFPPKPKDTLMISRTPEVTCVVVDV |
| | CD47)-CL | SHEDPEVKFNWYVDGVEVHNAKTKPREEQYNSTYRVVSVLTVLHQDWLNGKEYKCKVSNKALPAPIE |
| | (L = | KTISKAKGQPREPQVYTLPPSRDELTKNQVSLTCLVKGFYPSDIAVEWESNGQPENNYKTTPPVLDS |
| | (G4S)4) | DGSFFLYSKLTVDKSRWQQGNVFSCSVMHEALHNHYTQKSLSLSPGAGGGGSGGGGSGGGGSGGGGS |
| | | GDIVMTQSPATLSVTPGDRVSLSCRASQTISDYLHWYQQKSHESPRLLIKFASQSISGIPSRFSGSG |
| | | SGSDFTLSINSVEPEDVGVYYCQNGHGFPRTFGGGTKLEIKRGTVAAPSVFIFPPSDEQLKSGTASV |
| | | VCLLNNFYPREAKVQWKVDNALQSGNSQESVTEQDSKDSTYSLSSTLTLSKADYEKHKVYACEVTHQ |
| | | GLSSPVTKSFNRGEC |
| 81 | VH(anti- | atggagttgcctgttaggctgttggtgctgatgttctggattcctgctagctccagcGAGGTGCAGC |
| | CD47)-CH1- | TGGTGGAGTCTGGGGGAGACTTAGTGAAGCCTGGAGGGTCCCTGAAACTCTCCTGTGCAGCCTCTGG |
| | H-CH2-CH3- | ATTCACTTTCAGTGGCTATGGCATGTCTTGGGTTCGCCAGACTCCAGACAAGAGGCTGGAGTGGGTC |
| | L-VL(anti- | GCAACCATTACTAGTGGTGGTACTTACACCTACTATCCAGACAGTGTGAAGGGGCGATTCACCATCT |
| | EGFR)-CL | CCAGAGACAATGCCAAGAACACCCTGTACCTGCAAATAGACAGTCTGAAGTCTGAGGATACAGCCAT |
| | (L = | ATATTTCTGTGCAAGATCCCTCGCGGGAAATGCTATGGACTACTGGGGTCAAGGGACCAGCGTCACC |
| | (G4S)4) | GTCTCCTCAgcctccaccaagggcccatcggtcttccccctggcaccctcctccaagagcacctctg |
| | | ggggcacagcggccctgggctgcctggtcaaggactacttccccgaaccggtgacggtgtcgtggaa |
| | | ctcaggcgccctgaccagcggcgtgcacaccttcccggctgtcctacagtcctcaggactctactcc |
| | | ctcagcagcgtggtgaccgtgccctccagcagcttgggcacccagacctacatctgcaacgtgaatc |
| | | acaagcccagcaacaccaaggtggacaagagagttgagcccaaatcttgtgacaaaactcacacatg |
| | | cccaccgtgcccagcacctccagtggccggaccgtcagtcttcctcttccccccaaaacccaaggac |
| | | accctcatgatctcccggacccctgaggtcacatgcgtggtggtggacgtgagccacgaagaccctg |
| | | aggtcaagttcaactggtacgtggacggcgtggaggtgcataatgccaagacaaagccgcgggagga |
| | | gcagtacaacagcacgtaccgtgtggtcagcgtcctcaccgtcctgcaccaggactggctgaatggc |
| | | aaggagtacaagtgcaaggtctccaacaaagccctcccatccagcatcgagaaaaccatctccaaag |
| | | ccaaagggcagccccgagaaccacaggtgtacaccctgcccccatcacgggaggagatgaccaagaa |
| | | ccaggtcagcctgacctgcctggtcaaaggcttctatcccagcgacatcgccgtggagtgggagagc |
| | | aatgggcagccggagaacaactacaagaccacgcctcccgtgctggactccgacggctccttcttcc |
| | | tctatagcaagctcaccgtggacaagagcaggtggcagcaggggaacgtcttctcatgctccgtgat |
| | | gcatgaggctctgcacaaccactacacacagaagagcctctccctgtccccgggtgcaggtggaggt |
| | | gggagcGACATCTTGCTGACTCAGTCTCCAGTCATCCTGTCTGTGAGTCCAGGAGAAAGAGTCAGTT |
| | | TCTCCTGCAGGGCCAGTCAGAGTATTGGCACAAACATACACTGGTATCAGCAAAGAACAAATGGTTC |
| | | TCCAAGGCTTCTCATAAAGTATGCTTCTGAGTCTATCTCTGGAATCCCTTCCAGGTTTAGTGGCAGT |
| | | GGATCAGGGACAGATTTTACTCTTAGCATCAACAGTGTGGAGTCTGAAGATATTGCAGATTATTACT |
| | | GTCAACAAAATAATAACTGGCCAACCACGTTCGGTGCTGGGACCAAGCTGGAGCTGAAAcgaactgt |
| | | ggctgcaccatctgtcttcatcttcccgccatctgatgagcagttgaaatctggaactgcctctgtt |
| | | gtgtgcctgctgaataacttctatcccagagaggccaaagtacagtggaaggtggataacgccctcc |
| | | aatcgggtaactcccaggagagtgtcacagagcaggacagcaaggacagcacctacagcctcagcag |
| | | caccctgacgctgagcaaagcagactacgagaaacacaaagtctacgcctgcgaagtcacccatcag |
| | | ggcctgagctcgcccgtcacaaagagcttcaacaggggagagtgttga |
| 82 | VH(anti- | EVQLVESGGDLVKPGGSLKLSCAASGFTFSGYGMSWVRQTPDKRLEWVATITSGGTYTYYPDSVKGR |
| | CD47)-CH1- | FTISRDNAKNTLYLQIDSLKSEDTAIYFCARSLAGNAMDYWGQGTSVTVSSASTKGPSVFPLAPSSK |
| | H-CH2-CH3- | STSGGTAALGCLVKDYFPEPVTVSWNSGALTSGVHTFPAVLQSSGLYSLSSVVTVPSSSLGTQTYIC |
| | L-VL(anti- | NVNHKPSNTKVDKRVEPKSCDKTHTCPPCPAPPVAGPSVFLFPPKPKDTLMISRTPEVTCVVVDVSH |
| | EGFR)-CL | EDPEVKFNWYVDGVEVHNAKTKPREEQYNSTYRVVSVLTVLHQDWLNGKEYKCKVSNKALPSSIEKT |
| | (L = | ISKAKGQPREPQVYTLPPSREEMTKNQVSLICLVKGFYPSDIAVEWESNGQPENNYKTTPPVLDSDG |
| | (G4S)4) | SFFLYSKLTVDKSRWQQGNVFSCSVMHEALHNHYTQKSLSLSPGAGGGGSDILLTQSPVILSVSPGE |
| | | RVSFSCRASQSIGTNIHWYQQRTNGSPRLLIKYASESISGIPSRFSGSGSGTDFTLSINSVESEDIA |
| | | DYYCQQNNNWPTTFGAGTKLELKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSSPVTKSFNRGEC |
| 83 | anti-HER2 | gatattgtgatgactcagtctccagccaccctgtctgtgactccaggagatagagtctctctttcct |
| | Fab light | gcagggccagccagactattagcgactacttacactggtatcaacaaaaatcacatgagtctccaag |
| | chain | gcttctcatcaaatttgcttcccaatccatttctggaatcccctccaggttcagtggcagtggatca |
| | | ggctcagatttcactctcagtatcaacagtgtggaacctgaagatgttggagtgtattactgtcaaa |
| | | atggtcacggctttcctcggacgttcggtggagggaccaagctggaaataaaacgtggaactgtggc |
| | | tgcaccatctgtcttcatcttcccgccatctgatgagcagttgaaatctggaactgcctctgttgtg |
| | | tgcctgctgaataacttctatcccagagaggccaaagtacagtggaaggtggataacgccctccaat |
| | | cgggtaactcccaggagagtgtcacagagcaggacagcaaggacagcacctacagcctcagcagcac |
| | | cctgacgctgagcaaagcagactacgagaaacacaaagtctacgcctgcgaagtcacccatcagggc |
| | | ctgagctcgcccgtcacaaagagcttcaacaggggagag |
| 84 | anti-HER2 | DIVMTQSPATLSVTPGDRVSLSCRASQTISDYLHWYQQKSHESPRLLIKFASQSISGIPSRFSGSGS |
| | Fab light | GSDFTLSINSVEPEDVGVYYCQNGHGFPRTFGGGTKLEIKRGTVAAPSVFIFPPSDEQLKSGTASVV |
| | chain | CLLNNFYPREAKVQWKVDNALQSGNSQESVTEQDSKDSTYSLSSTLTLSKADYEKHKVYACEVTHQG |
| | | LSSPVTKSFNRGEC |
| 85 | anti-HER2 | GAGGTGCAGCTGGTGGAGTCTGGGGGAGACTTAGTGAAGCCTGGAGGGTCCCTGAAACTCTCCTGTG |
| | Fab heavy | CAGCCTCTGGATTCACTTTCAGTGGCTATGGCATGTCTTGGGTTCGCCAGACTCCAGACAAGAGGCT |
| | chain | GGAGTGGGTCGCAACCATTACTAGTGGTGGTACTTACACCTACTATCCAGACAGTGTGAAGGGGCGA |
| | | TTCACCATCTCCAGAGACAATGCCAAGAACACCCTGTACCTGCAAATAGACAGTCTGAAGTCTGAGG |
| | | ATACAGCCATATATTTCTGTGCAAGATCCCTCGCGGGAAATGCTATGGACTACTGGGGTCAAGGGAC |
| | | CAGCGTCACCGTCTCCTCAGCTAGCACCAAGGGCCCATCGGTCTTCCCCCTGGCACCCTCCTCCAAG |
| | | AGCACCTCTGGGGGCACAGCGGCCCTGGGCTGCCTGGTCAAGGACTACTTCCCCGAACCGGTGACGG |
| | | TGTCGTGGAACTCAGGCGCCCTGACCAGCGGCGTGCACACCTTCCCGGCTGTCCTACAGTCCTCAGG |
| | | ACTCTACTCCCTCAGCAGCGTGGTGACCGTGCCCTCCAGCAGCTTGGGCACCCAGACCTACATCTGC |
| | | AACGTGAATCACAAGCCCAGCAACACCAAGGTGGACAAGAAAGTTGAGCCCAAATCT |
| 86 | anti-HER2 | EVQLVESGGDLVKPGGSLKLSCAASGFTFSGYGMSWVRQTPDKRLEWVATITSGGTYTYYPDSVKGR |
| | Fab heavy | FTISRDNAKNTLYLQIDSLKSEDTAIYFCARSLAGNAMDYWGQGTSVTVSSASTKGPSVFPLAPSSK |
| | chain | STSGGTAALGCLVKDYFPEPVTVSWNSGALTSGVHTFPAVLQSSGLYSLSSVVTVPSSSLGTQTYIC |
| | | NVNHKPSNTKVDKRVEPKSC |
| 87 | VH(anti- | atggagttgcctgttaggctgttggtgctgatgttctggattcctgctagctccagcgaggtgcaac |
| | HER2)-CH1- | tggtggagagcggaggaggcctcgtgcaacccggaggatccctcagactgagctgtgccgccagcgg |
| | H-CH2-CH3- | cttcaatatcaaggatacctatatccactgggtgaggcaggcccccggaaaaggactggagtgggtg |
| | L-VL(anti- | gccaggatctatcccacaaacggctacaccaggtacgccgattccgtgaagggcagattcaccatca |
| | CD47)-CL | gcgccgatacctccaaaaacaccgcctatctccagatgaacagcctcagagccgaggacaccgccgt |
| | (L = | ctattactgttccagatggggcggcgacggcttttacgctatggattactggggccagggaaccctg |
| | (G4S)4) | gtgaccgtgagcagcgcctccaccaagggcccatcggtcttccccctggcaccctcctccaagagca |
| | | cctctgggggcacagcggccctgggctgcctggtcaaggactacttccccgaaccggtgacggtgtc |
| | | gtggaactcaggcgccctgaccagcggcgtgcacaccttcccggctgtcctacagtcctcaggactc |
| | | tactccctcagcagcgtggtgaccgtgccctccagcagcttgggcacccagacctacatctgcaacg |
| | | tgaatcacaagcccagcaacaccaaggtggacaagaaagttgagcccaaatcttgtgacaaaactca |
| | | cacatgcccaccgtgcccagcacctgaactcctggggggaccgtcagtcttcctcttccccccaaaa |
| | | cccaaggacaccctcatgatctcccggacccctgaggtcacatgcgtggtggtggacgtgagccacg |
| | | aagaccctgaggtcaagttcaactggtacgtggacggcgtggaggtgcataatgccaagacaaagcc |
| | | gcgggaggagcagtacaacagcacgtaccgtgtggtcagcgtcctcaccgtcctgcaccaggactgg |
| | | ctgaatggcaaggagtacaagtgcaaggtctccaacaaagccctcccagcccccatcgagaaaacca |
| | | tctccaaagccaaagggcagccccgagaaccacaggtgtacaccctgcccccatcacgggatgagct |
| | | gaccaagaaccaggtcagcctgacctgcctggtcaaaggcttctatcccagcgacatcgccgtggag |
| | | tgggagagcaatgggcagccggagaacaactacaagaccacgcctcccgtgctggactccgacggct |
| | | ccttcttcctctatagcaagctcaccgtggacaagagcaggtggcagcaggggaacgtcttctcatg |
| | | ctccgtgatgcatgaggctctgcacaaccactacacgcagaagagcctctccctgtccccgggtgct |
| | | ggcggcggaggaagcggaggaggaggcagcggaggcggaggctccggcggaggaggctccggagata |
| | | ttgtgatgactcagtctccagccaccctgtctgtgactccaggagataaagtctctctttcctgcag |
| | | ggccagccagactattagcgactacttacactggtatcaacaaaaatcacatgagtctccaaggctt |
| | | ctcatcaaatttgcttcccaatccatttctggaatcccctccaggttcagtggcagtggatcaggct |
| | | cagatttcactctcagtatcaacagtgtggaacctgaagatgttggagtgtattactgtcaaaatgg |
| | | tcacggctttcctcggacgttcggtggagggaccaagctggaaataaaacgtggaactgtggctgca |
| | | ccatctgtcttcatcttcccgccatctgatgagcagttgaaatctggaactgcctctgttgtgtgcc |
| | | tgctgaataacttctatcccagagaggccaaagtacagtggaaggtggataacgccctccaatcggg |
| | | taactcccaggagagtgtcacagagcaggacagcaaggacagcacctacagcctcagcagcaccctg |
| | | acgctgagcaaagcagactacgagaaacacaaagtctacgcctgcgaagtcacccatcagggcctga |
| | | gctcgcccgtcacaaagagcttcaacaggggagagtgttga |
| 88 | VL(anti- | ATGGAGTTGCCTGTTAGGCTGTTGGTGCTGATGTTCTGGATTCCTGCTTCCTTAAGCgacatccaga |
| | HER2)-CL | tgacccagagccctagcagcctgagcgcgagcgtgggcgacagagtgacaatcacctgcagggccag |
| | | ccaggacgtgaataccgccgtggcctggtaccagcagaaacccggcaaggcccctaagctgctgatc |
| | | tactccgcctccttcctctacagcggcgtgcccagcaggtttagcggcagcaggagcggcacagatt |
| | | tcaccctgaccatcagcagcctgcagcccgaggacttcgccacctactactgccagcagcattacac |
| | | caccccccccaccttcggccagggaacaaaggtaaaaatcaagCGAACTGTGGCTGCACCATCTGTC |
| | | TTCATCTTCCCGCCATCTGATGAGCAGTTGAAATCTGGAACTGCCTCTGTTGTGTGCCTGCTGAATA |
| | | ACTTCTATCCCAGAGAGGCCAAAGTACAGTGGAAGGTGGATAACGCCCTCCAATCGGGTAACTCCCA |
| | | GGAGAGTGTCACAGAGCAGGACAGCAAGGACAGCACCTACAGCCTCAGCAGCACCCTGACGCTGAGC |
| | | AAAGCAGACTACGAGAAACACAAAGTCTACGCCTGCGAAGTCACCCATCAGGGCCTGAGCTCGCCCG |
| | | TCACAAAGAGCTTCAACAGGGGAGAGTGTTAG |
| 89 | VH(anti- | EVQLVESGGDLVKPGGSLKLSCAASGFTFSGYGMSWVRQTPDKRLEWVATITSGGTYTYYPDSVKGR |
| | HER2)-CH1- | FTISRDNAKNTLYLQIDSLKSEDTAIYFCARSLAGNAMDYWGQGTSVTVSSASTKGPSVFPLAPSSK |
| | H-CH2-CH3- | STSGGTAALGCLVKDYFPEPVTVSWNSGALTSGVHTFPAVLQSSGLYSLSSVVTVPSSSLGTQTYIC |
| | L-VL(anti- | NVNHKPSNTKVDKKVEPKSCDKTHTCPPCPAPELLGGPSVFLFPPKPKDTLMISRTPEVTCVVVDVS |
| | CD47)-CL | HEDPEVKFNWYVDGVEVHNAKTKPREEQYNSTYRVVSVLTVLHQDWLNGKEYKCKVSNKALPAPIEK |
| | (L = | TISKAKGQPREPQVYTLPPSRDELTKNQVSLTCLVKGFYPSDIAVEWESNGQPENNYKTTPPVLDSD |
| | (G4S)4) | GSFFLYSKLTVDKSRWQQGNVFSCSVMHEALHNHYTQKSLSLSPGAGGGGSGGGGSGGGGSGGGGSG |
| | | DIVMTQSPATLSVTPGDRVSLSCRASQTISDYLHWYQQKSHESPRLLIKFASQSISGIPSRFSGSGS |
| | | GSDFTLSINSVEPEDVGVYYCQNGHGFPRTFGGGTKLEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 90 | VL(anti- | DIVMTQSPATLSVTPGDRVSLSCRASQTISDYLHWYQQKSHESPRLLIKFASQSISGIPSRFSGSGS |
| | HER2)-CL | GSDFTLSINSVEPEDVGVYYCQNGHGFPRTFGGGTKLEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGL |
| | | SSPVTKSFNRGEC |
| 91 | VH(anti- | atggagttgcctgttaggctgttggtgctgatgttctggattcctgctagctccagcGAGGTGCAGC |
| | CD47)-CH1- | TGGTGGAGTCTGGGGGAGACTTAGTGAAGCCTGGAGGGTCCCTGAAACTCTCCTGTGCAGCCTCTGG |
| | H-CH2-CH3- | ATTCACTTTCAGTGGCTATGGCATGTCTTGGGTTCGCCAGACTCCAGACAAGAGGCTGGAGTGGGTC |
| | L-VL(anti- | GCAACCATTACTAGTGGTGGTACTTACACCTACTATCCAGACAGTGTGAAGGGGCGATTCACCATCT |
| | HER2)-CL | CCAGAGACAATGCCAAGAACACCCTGTACCTGCAAATAGACAGTCTGAAGTCTGAGGATACAGCCAT |
| | (L = | ATATTTCTGTGCAAGATCCCTCGCGGGAAATGCTATGGACTACTGGGGTCAAGGGACCAGCGTCACC |
| | (G4S)4) | GTCTCCTCAgcctccaccaagggcccatcggtcttccccctggcaccctcctccaagagcacctctg |
| | | ggggcacagcggccctgggctgcctggtcaaggactacttccccgaaccggtgacggtgtcgtggaa |
| | | ctcaggcgccctgaccagcggcgtgcacaccttcccggctgtcctacagtcctcaggactctactcc |
| | | ctcagcagcgtggtgaccgtgccctccagcagcttgggcacccagacctacatctgcaacgtgaatc |
| | | acaagcccagcaacaccaaggtggacaagagagttgagcccaaatcttgtgacaaaactcacacatg |
| | | cccaccgtgcccagcacctccagtggccggaccgtcagtcttcctcttccccccaaaacccaaggac |
| | | accctcatgatctcccggacccctgaggtcacatgcgtggtggtggacgtgagccacgaagaccctg |
| | | aggtcaagttcaactggtacgtggacggcgtggaggtgcataatgccaagacaaagccgcgggagga |
| | | gcagtacaacagcacgtaccgtgtggtcagcgtcctcaccgtcctgcaccaggactggctgaatggc |
| | | aaggagtacaagtgcaaggtctccaacaaagccctcccatccagcatcgagaaaaccatctccaaag |
| | | ccaaagggcagccccgagaaccacaggtgtacaccctgcccccatcacgggaggagatgaccaagaa |
| | | ccaggtcagcctgacctgcctggtcaaaggcttctatcccagcgacatcgccgtggagtgggagagc |
| | | aatgggcagccggagaacaactacaagaccacgcctcccgtgctggactccgacggctccttcttcc |
| | | tctatagcaagctcaccgtggacaagagcaggtggcagcaggggaacgtcttctcatgctccgtgat |
| | | gcatgaggctctgcacaaccactacacacagaagagcctctccctgtccccgggtgcaggtggaggt |
| | | gggagcgacatccagatgacccagagccctagcagcctgagcgcgagcgtgggcgacagagtgacaa |
| | | tcacctgcagggccagccaggacgtgaataccgccgtggcctggtaccagcagaaacccggcaaggc |
| | | ccctaagctgctgatctactccgcctccttcctctacagcggcgtgcccagcaggtttagcggcagc |
| | | aggagcggcacagatttcaccctgaccatcagcagcctgcagcccgaggacttcgccacctactact |
| | | gccagcagcattacaccaccccccccaccttcggccagggaacaaaggtggagatcaagcgaactgt |
| | | ggctgcaccatctgtcttcatcttcccgccatctgatgagcagttgaaatctggaactgcctctgtt |
| | | gtgtgcctgctgaataacttctatcccagagaggccaaagtacagtggaaggtggataacgccctcc |
| | | aatcgggtaactcccaggagagtgtcacagagcaggacagcaaggacagcacctacagcctcagcag |
| | | caccctgacgctgagcaaagcagactacgagaaacacaaagtctacgcctgcgaagtcacccatcag |
| | | ggcctgagctcgcccgtcacaaagagcttcaacaggggagagtgttga |
| 92 | VH(anti- | ATGGAGTTGCCTGTTAGGCTGTTGGTGCTGATGTTCTGGATTCCTGCTTCCAGCAGCgaggtgcaac |
| | HER2)-CH1- | tggtggagagcggaggaggcctcgtgcaacccggaggatccctcagactgagctgtgccgccagcgg |
| | H* | cttcaatatcaaggatacctatatccactgggtgaggcaggcccccggaaaaggactggagtgggtg |
| | | gccaggatctatcccacaaacggctacaccaggtacgccgattccgtgaagggcagattcaccatca |
| | | gcgccgatacctccaaaaacaccgcctatctccagatgaacagcctcagagccgaggacaccgccgt |
| | | ctattactgttccagatggggcggcgacggcttttacgctatggattactggggccagggaaccctg |
| | | gtgaccgtgagcagcGCTAGCACCAAGGGCCCATCGGTCTTCCCCCTGGCACCCTCCTCCAAGAGCA |
| | | CCTCTGGGGGCACAGCGGCCCTGGGCTGCCTGGTCAAGGACTACTTCCCCGAACCGGTGACGGTGTC |
| | | GTGGAACTCAGGCGCCCTGACCAGCGGCGTGCACACCTTCCCGGCTGTCCTACAGTCCTCAGGACTC |
| | | TACTCCCTCAGCAGCGTGGTGACCGTGCCCTCCAGCAGCTTGGGCACCCAGACCTACATCTGCGCCG |
| | | TGATCAGCAGCGGCGGCAGCTCCATCAACTACAAGAAAGTTGAGCCCAAATCTTGTTAA |
| 93 | VH(anti- | EVQLVESGGDLVKPGGSLKLSCAASGFTFSGYGMSWVRQTPDKRLEWVATITSGGTYTYYPDSVKGR |
| | CD47)CH1- | FTISRDNAKNTLYLQIDSLKSEDTAIYFCARSLAGNAMDYWGQGTSVTVSSASTKGPSVFPLAPSSK |
| | H-CH2-CH3- | STSGGTAALGCLVKDYFPEPVTVSWNSGALTSGVHTFPAVLQSSGLYSLSSVVTVPSSSLGTQTYIC |
| | L-VL(anti- | NVNHKPSNTKVDKRVEPKSCDKTHTCPPCPAPPVAGPSVFLFPPKPKDTLMISRTPEVTCVVVDVSH |
| | HER2)-CL | EDPEVKFNWYVDGVEVHNAKTKPREEQYNSTYRVVSVLTVLHQDWLNGKEYKCKVSNKALPSSIEKT |
| | (L = | ISKAKGQPREPQVYTLPPSREEMTKNQVSLTCLVKGFYPSDIAVEWESNGQPENNYKTTPPVLDSDG |
| | (G4S)4) | SFFLYSKLTVDKSRWQQGNVFSCSVMHEALHNHYTQKSLSLSPGAGGGGSDIVMTQSPATLSVTPGD |
| | | RVSLSCRASQTISDYLHWYQQKSHESPRLLIKFASQSISGIPSRFSGSGSGSDFTLSINSVEPEDVG |
| | | VYYCQNGHGFPRTFGGGTKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLSSPVTKSFNRGEC |
| 94 | VH(anti- | EVQLVESGGDLVKPGGSLKLSCAASGFTFSGYGMSWVRQTPDKRLEWVATITSGGTYTYYPDSVKGR |
| | HER2)-CH1- | FTISRDNAKNTLYLQIDSLKSEDTAIYFCARSLAGNAMDYWGQGTSVTVSSASTKGPSVFPLAPSSK |
| | H* | STSGGTAALGCLVKDYFPEPVTVSWNSGALTSGVHTFPAVLQSSGLYSLSSVVTVPSSSLGTQTYIC |
| | | NVNHKPSNTKVDKKVEPKSC |

### Example 1

### Fc-Anti-EGFR Precursor Molecules

1A) Construction and Expression of Fc-Fab Precursors

In order to create full TetBiAb molecules, a number of Fc-Fab precursors were generated and tested to see if antigen binding of the Fab can still occur when the Fab is moved to the C-terminus of Fc. The generation of the Fc-anti-EGFR is based on the anti-EGFR C225 (cetuximab) monoclonal antibody (Kawamoto, PNAS 80:1337, 1983). The DNA and protein sequence of the Fab light chain for C225 are provided in SEQ ID NO:1 and SEQ ID NO:2, respectively. The DNA and protein sequence of the Fab heavy chain for C225 are provided in SEQ ID NO:3 and SEQ ID NO:4, respectively. Three different Fc-EGFR molecules were generated: (i) Fc-G4S-anti-EGFR(VHCH1), in which the C-terminus of the Fc region heavy chain is linked to the N-terminus of the anti-EGFR Fab heavy chain via a G4S linker (GGGGS, heavy chain is linked to the N-terminus of the anti-EGFR Fab light chain via a G4S linker; and (iii) Fc-(G4S)₄-anti-EGFR(LC), which is the same molecule as (ii) but with a quadruple repeat of the linker.

For expression of Fc-G4S-anti-EGFR(VHCH1), the following two gene constructs were assembled by standard recombinant DNA techniques and cloned into the mammalian expression vector pTT5 (containing the mouse light chain signal peptide sequence for secretion): 1) Construct H-CH2-CH3-G4S-VH(anti-EGFR)-CH1-H (SEQ ID NO:11), encoding the following elements: a human heavy chain hinge region with cysteine (which natively forms a disulfide bond with the light chain) mutated to a serine, (EPKSS, SEQ ID NO:8), followed by constant domains 2 and 3, followed by a G4S linker, and anti-EGFR heavy chain variable domain followed by human heavy chain constant domain 1 followed by the hinge region (EPKSC, SEQ ID NO:10, to allow for a disulfide bridge with the anti-EGFR light chain); and 2) Construct VL(anti-EGFR)-CL (SEQ ID NO:12), encoding the following elements: an anti-EGFR light chain variable domain followed by human kappa light chain constant domain. The corresponding amino acid sequences for these two constructs are shown in SEQ ID NO:13 and SEQ ID NO:14 respectively.

For expression of Fc-G4S-anti-EGFR(LC), the following two gene constructs were assembled by standard recombinant DNA techniques and cloned into the mammalian expression vector pTT5 (containing the mouse light chain signal peptide sequence for secretion): 1) Construct H-CH2-CH3-G4S-VL(anti-EGFR)-CL (SEQ ID NO:15), encoding the following elements: a human heavy chain hinge region EPKSC (SEQ ID NO:8) followed by constant domains 2 and 3, followed by a G4S linker, and anti-EGFR light chain variable domain followed by human kappa light chain constant domain; and 2) Construct VH(anti-EGFR)-CH1-H (SEQ ID NO:16), encoding the following elements: anti-EGFR heavy chain variable domain followed by human heavy chain constant domain 1 followed by the hinge region EPKSC (SEQ ID NO:10). The corresponding amino acid sequences for these two constructs are shown in SEQ ID NO:17 and SEQ ID NO:18 respectively.

For expression of Fc-(G4S)₄-anti-EGFR(LC), the following two gene constructs were assembled by standard recombinant DNA techniques and cloned into the mammalian expression vector pTT5 (containing the mouse light chain signal peptide sequence for secretion): 1) Construct H-CH2-CH3-(G4S)₄-VL(anti-EGFR)-CL (SEQ ID NO:19), encoding the following elements: a human heavy chain hinge region EPKSC (SEQ ID NO:8) followed by constant domains 2 and 3, followed by a (G4S)₄ linker, and anti-EGFR light chain variable domain followed by human kappa light chain constant domain; and 2) Construct VH(anti-EGFR)-CH1-H (SEQ ID NO:16), encoding the following elements: anti-EGFR heavy chain variable domain followed by human heavy chain constant 1 followed by the hinge region EPKSC (SEQ ID NO:10). The corresponding amino acid sequences for these two constructs are shown in SEQ ID NO:20 and SEQ ID NO:18 respectively.

Each set of the two vectors was co-transfected transiently into HEK 293-6E cells using Genejuice (Life Technologies, Grand Island, N.Y.) or polyethylenimine (PEI, Polysciences, Warrington, Pa.) for expression of Fc-G4S-anti-EGFR(VHCH1), Fc-G4S-anti-EGFR(LC), and Fc-G4S₄-anti-EGFR(LC). The proteins were purified in a single step by protein A affinity chromatography. Expression of the two polypeptides and assembly of the full tetrameric molecule were confirmed on sodium dodecyl sulfate-polyacrylamide gel electrophoresis (SDS-PAGE) and size exclusion chromatography (SEC).

In addition, a control anti-EGFR in a standard monoclonal antibody format (anti-EGFR IgG1) was generated to compare to the different Fc-Fab formats.

1B) Binding of Fc-Fab Precursors to Antigens

1Bi) Competition Binding of Fc-EGFR for EGF on A431 Membranes

The ability of Fc-G4S-anti-EGFR(VHCH1) and Fc-G4S-anti-EGFR(LC) to retain binding for EGFR was shown by competitive radioligand binding assays. Competing antibodies were mixed with ¹²⁵I-EGF (Perkin Elmer, Waltham, Mass.) prior to the addition of 2 mg of membrane prepared from human A431 epidermoid carcinoma cells that overexpress EGFR. A431 cell membranes were prepared by nitrogen cavitation. The cells were disrupted with 900 psi of with N₂ gas for 30 min, after which the lysate was centrifuged at 1000 g for 10 min at 4° C. The supernatant was collected and centrifuged at 100,000 g for 1 h at 4° C. The resulting pellet was re-suspended with a dounce homogenizer. The protein concentration of the samples was determined using the BioRad protein assay reagent, and the samples were stored frozen at −80° C. for future use. Non-specific binding was determined in the presence of a large excess of unlabeled EGF (100 nM) to saturate all the EGFR binding sites. The reactions were incubated for 90 min at 37° C., with shaking, and terminated by filtering through glass fiber filters (EMD Millipore, Billerica, Mass.). The filters were washed and counted on a gamma counter to determine the amount of ¹²⁵I-EGF bound on the A431 cell memebrane.

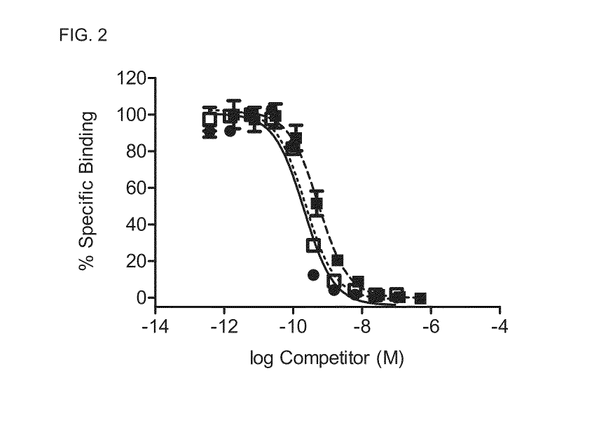

The results show that Fc-G4S-anti-EGFR(VHCH1) has a similar ability to inhibit binding of ¹²⁵I-EGF to EGFR on A431 cell membranes as anti-EGFR (FIG. 2). Fc-G4S-anti-EGFR(LC) also bound to EGFR, although with a slightly higher inhibition constant (Ki) (FIG. 2). These results demonstrate that anti-EGFR Fab fused to the C-terminus of Fc via the N-terminus of either VH or VL retained binding to EGFR.

1Bii) SPR Analysis

The ability for Fc-G4S-anti-EGFR(VHCH1), Fc-G4S-anti-EGFR(LC), and Fc-(G4S)₄-anti-EGFR(LC) to retain binding for EGFR was determined by surface plasmon resonance (SPR). Purified goat anti-human IgG Fc (Jackson Immuno Research Laboratories) was immobilized onto the CM5 chip using amine coupling chemistry using a Biacore 4000 instrument (GE Healthcare). Biacore CM-5 chips, ethanolamine, NHS/EDC coupling reagents and buffers were obtained from Biacore (GE Healthcare). The immobilization steps were carried out at a flow rate of 30 μl/min in HEPES buffer (20 mM HEPES, 150 mM NaCl, 3.4 mM EDTA and 0.005% P20 surfactant). The sensor surfaces were activated for 7 min with a mixture of NHS (0.05 M) and EDC (0.2 M). The goat anti-human IgG Fc was injected at a concentration of ˜30 μg/ml in 10 mM sodium acetate, pH 5.0, for 7 min. Ethanolamine (1 M, pH 8.5) was injected for 7 min to block any remaining activated groups. An average of 12,000 response units (RU) of capture antibody was immobilized on each flow cell. Kinetic binding experiments were performed using the same HEPES buffer (20 mM HEPES, 150 mM NaCl, 3.4 mM EDTA and 0.005% P20 surfactant) and was equilibrated at 25° C. Kinetic data was collected by injecting test and control antibodies at 0.5 and 1 μg/ml for two minutes at a flow rate of 30 μl/min, followed by a buffer wash for 30 s at the same flow rate. Human EGFR-1 (R&D Systems recombinant Human EGF Receptor (1095-ER)) was bound at 40, 20, 10, 5, 2.5 and 0 nM for 3 min followed by a dissociation step for 10 min at the 30 μl/min flow rate. The data were fit using a 1:1 Langmuir binding model with the BIA evaluation software. Kinetic rate constants were determined from the fits of the association and dissociation phases, and the K_{D} was derived from the ratio of these constants.

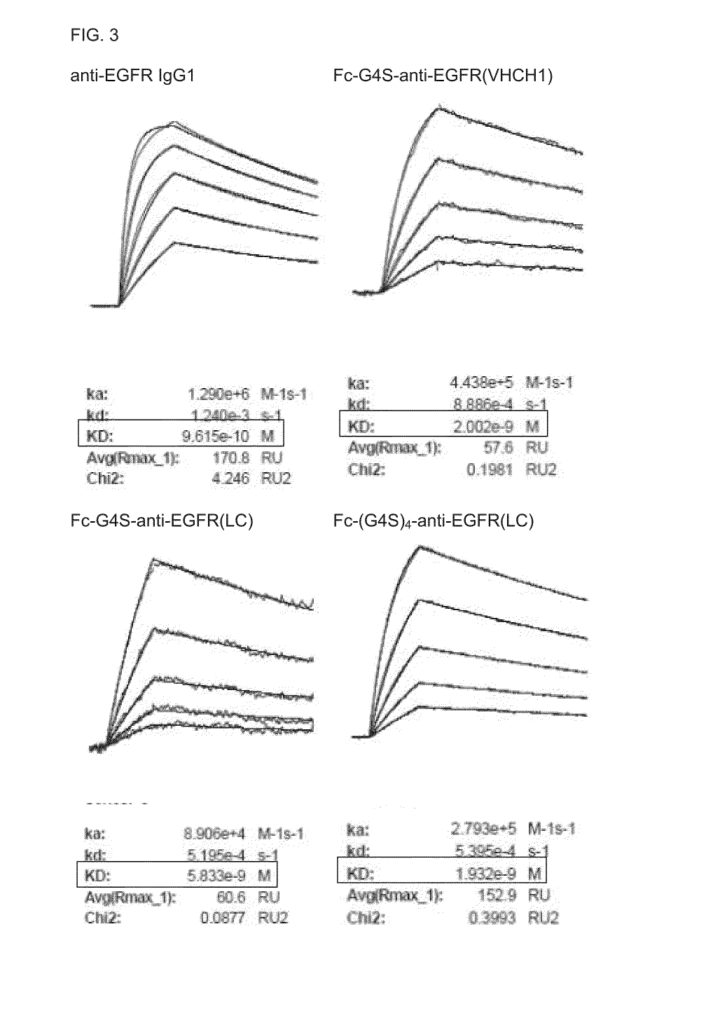

The results show that Fc-G4S-anti-EGFR(VHCH1) bound EGFR with a slightly higher K_{D} than anti-EGFR, ˜2 nM vs ˜1 nM respectively (FIG. 3). Fc-G4S-anti-EGFR(LC) also bound to EGFR, but with a KD of ˜6 nM (FIG. 3). When the linker was lengthened to (G4S)₄, the K_{D} of Fc-(G4S)₄-anti-EGFR(LC) dropped to ˜2 nM (FIG. 3). These results differ from the competition binding assay in part 1Bi and one possible explanation may be the lack of accessibility to the anti-EGFR Fab when the Fc-anti-EGFR is captured on the Biacore chip via anti-Fc. This hypothesis is supported by the increase in affinity when the linker was lengthened, potentially by increasing accessibility of anti-EGFR Fab for binding to EGFR.

### Example 2

### Fo-Anti-CD20 Precursor Molecules

2A) Construction and Expression of Fc-Fab Precursors

The generation of Fc-anti-CD20 is based on the anti-CD20 2B8 (rituximab) monoclonal antibody (Reff et al, Blood 83:435, 1994). The DNA and protein sequence of the Fab light chain for 2B8 are provided in SEQ ID NO:21 and SEQ ID NO:22, respectively. The DNA and protein sequence of the Fab heavy chain for 2B8 are provided in SEQ ID NO:23 and SEQ ID NO:24, respectively. Four different Fc-CD20 molecules were generated: (i) Fc-G4S-anti-CD20(VHCH1), in which the C-terminus of the Fc region eavy chain is linked to the N-terminus of the anti-CD20 Fab heavy chain via a G4S linker (GGGGS, SEQ ID NO:6); (ii) Fc-(G4S)₄-anti-CD20(VHCH1), which is the same molecule as (i) but with a quadruple repeat of the linker; (iii) Fc-G4S-anti-CD20(LC), in which the C-terminus of the Fc region heavy chain is linked to the N-terminus of the anti-CD20 Fab light chain via a G4S linker; and (iv) Fc-(G4S)₄-anti-CD20(LC), which is the same molecule as (iil) but with a quadruple repeat of the linker.

For expression of Fc-G4S-anti-CD20(VHCH1), the following two gene constructs were assembled by standard recombinant DNA techniques and cloned into the mammalian expression vector pTT5 (containing the mouse light chain signal peptide sequence for secretion): 1) Construct H-CH2-CH3-G4S-VH(anti-CD20)-CH1-H (SEQ ID NO:25), encoding the following elements: a human heavy chain hinge region EPKSC (SEQ ID NO:8) followed by constant domains 2 and 3, followed by a G4S linker, and anti-CD20 heavy chain variable domain followed by human heavy chain constant domain 1 followed by the hinge region EPKSC (SEQ ID NO:10); and 2) Construct VL(anti-CD20)-CL (SEQ ID NO:26), encoding the following elements: an anti-CD20 light chain variable domain followed by human kappa light chain constant domain:). The corresponding amino acid SEQ ID NO:for these two constructs are shown in SEQ ID NO:27 and SEQ ID NO:28 respectively.

For expression of Fc-(G4S)₄-anti-CD20(VHCH1), the following two gene constructs were assembled by standard recombinant DNA techniques and cloned into the mammalian expression vector pTT5 (containing the mouse light chain signal peptide sequence for secretion): 1) Construct H-CH2-CH3-(G4S)₄-VH(anti-CD20)-CH1-H (SEQ ID NO:29), encoding the following elements: a human heavy chain hinge region EPKSC (SEQ ID NO:8) followed by constant domains 2 and 3, followed by a (G4S)₄ linker, and anti-CD20 heavy chain variable domain followed by human heavy chain constant domain 1 followed by the hinge region EPKSC (SEQ ID NO:10); and 2) Construct VL(anti-CD20)-CL (SEQ ID NO:26), encoding the following elements: an anti-CD20 light chain variable domain followed by human kappa light chain constant domain:). The corresponding amino acid SEQ ID NO:for these two constructs are shown in SEQ ID NO:30 and SEQ ID NO:28 respectively.

For expression of Fc-G4S-anti-CD20(LC), the following two gene constructs were assembled by standard recombinant DNA techniques and cloned into the mammalian expression vector pTT5 (containing the mouse light chain signal peptide sequence for secretion): 1) Construct H-CH2-CH3-G4S-VL(anti-CD20)-CL (SEQ ID NO:31), encoding the following elements: a human heavy chain hinge region EPKSC (SEQ ID NO:8) followed by constant domains 2 and 3, followed by a G4S linker, and anti-CD20 light chain variable domain followed by human kappa light chain constant domain; and 2) Construct VH(anti-CD20)-CH1-H (SEQ ID NO:32), encoding the following elements: anti-CD20 heavy chain variable domain followed by human heavy chain constant domain 1 followed by the hinge region EPKSC (SEQ ID NO:10). The corresponding amino acid SEQ ID NO:for these two constructs are shown in SEQ ID NO:33 and SEQ ID NO:34 respectively.

For expression of Fc-(G4S)₄-anti-CD20(LC), the following two gene constructs were assembled by standard recombinant DNA techniques and cloned into the mammalian expression vector pTT5 (containing the mouse light chain signal peptide sequence for secretion): 1) Construct H-CH2-CH3-(G4S)₄-VL(anti-CD20)-CL (SEQ ID NO:35), encoding the following elements: a human heavy chain hinge region EPKSC (SEQ ID NO:8) followed by constant domains 2 and 3, followed by a (G4S)₄ linker, and anti-CD20 light chain variable domain followed by human kappa light chain constant domain; and 2) Construct VH(anti-CD20)-CH1-H (SEQ ID NO:32), encoding the following elements: anti-CD20 heavy chain variable domain followed by human heavy chain constant domain 1 followed by the hinge region EPKSC (SEQ ID NO:10). The corresponding amino acid SEQ ID NO:for these two constructs are shown in SEQ ID NO:36 and SEQ ID NO:34 respectively.

Each set of the two vectors was co-transfected transiently into HEK 293-6E cells using Genejuice (Life Technologies, Grand Island, N.Y.) or polyethylenimine (PEI, Polysciences, Warrington, Pa.) for expression of Fc-G4S-anti-CD20(VHCH1), Fc-(G4S)₄-anti-CD20(VHCH1), Fc-G4S-anti-CD20(LC), and Fc-(G4S)₄-anti-CD20(LC). The proteins were purified in a single step by protein A affinity chromatography. Expression of the two polypeptides and assembly of the full tetrameric molecule were confirmed on sodium dodecyl sulfate-polyacrylamide gel electrophoresis (SDS-PAGE) and size exclusion chromatography (SEC).

In addition, a control anti-CD20 in a standard monoclonal antibody format (anti-CD20 IgG1) was generated to compare to the differen Fc-Fab formats.

2B) Binding of Fc-Fab Precursors to Antigens

The ability of Fc-G4S-anti-CD20(VHCH1), Fc-G4S₄-anti-CD20(VHCH1), Fc-G4S-anti-CD20(LC), and Fc-G4S₄-anti-CD20(LC) to retain binding to CD20 on the cell surface was measured on human Daudi Burkitt's lymphoma cells, which express CD20. 1×10⁵ Daudi cells per well were incubated with varying concentrations of anti-CD20/anti-CD16 and anti-CD20 diluted in PBS+1% FBS in a 96 well plate for 30 min on ice. After washing with PBS+1% FBS, cells were incubated with TRITC F(ab′)2 goat Anti-Human IgG, Fcγ (Jackson ImmunoResearch, West Grove, Pa.), diluted 1:200 in PBS+1% FBS for 30 min on ice. After washing again, cells were fixed with 1% formaldehyde in PBS. Cells were analyzed by flow cytometry (Guava, EMD Millipore, Billerica, Mass.).

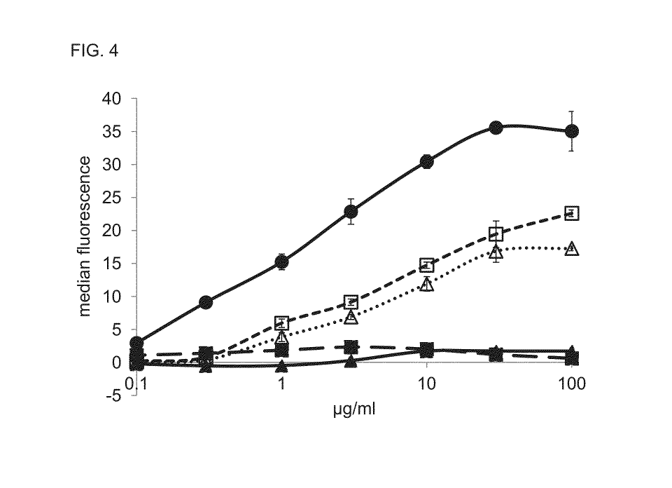

The results show that Fc-G4S-anti-CD20(VHCH1) and Fc-G4S₄-anti-CD20(VHCH1) retain binding to CD20, although not as well as anti-CD20 IgG1 (FIG. 4). Increasing the linker length appears to enhance the binding, but not to the same extent as anti-EGFR (FIG. 3). Neither Fc-G4S-anti-CD20(LC) nor Fc-G4S₄-anti-CD20(LC) retain binding to CD20 (FIG. 4). The anti-CD20 Fab binds poorly to CD20 expressed on cell membrane when it is attached at the C-terminus of Fc, especially when attached by VL. CD20 is a transmembrane protein and anti-CD20 only binds to an extracellular loop. The Fc likely hinders accessibility to C-terminal Fab to bind the small loop. Antibodies to larger extracellular domain, such as anti-EGFR, are better candidates for tetravalent bispecific antibodies.

### Example 3

### Anti-EGFR/Anti-CD16 and Anti-CD16/Anti-EGFR

3A) Construction and Expression of TetBiAbs

The generation of the TetBiAbs against EGFR and CD16 is based on the anti-EGFR C225 (cetuximab) monoclonal antibody (Kawamoto, PNAS 80:1337, 1983) and the anti-CD16 3G8 monoclonal antibody (Fleit et al, PNAS 79:3275, 1982). The DNA and protein sequence of the Fab light chain for C225 are provided in SEQ ID NO:1 and SEQ ID NO:2, respectively. The DNA and protein sequence of the Fab heavy chain for C225 are provided in SEQ ID NO:3 and SEQ ID NO:4, respectively. The DNA and protein sequence of the Fab light chain for 3G8 are provided in SEQ ID NO:37 and SEQ ID NO:38, respectively. The DNA and protein sequence of the Fab heavy chain for 3G8 are provided in SEQ ID NO:39 and SEQ ID NO:40, respectively. Two different TetBiAbs against EGFR and CD16 molecules were generated: (i) anti-EGFR/anti-CD16, in which the C-terminus of the anti-EGFR heavy chain polypeptide is linked to the N-terminus of the anti-CD16 Fab light chain via a G4S linker and (ii) anti-CD16/anti-EGFR, in which the C-terminus of the anti-CD16 heavy chain polypeptide is operably linked to the N-terminus of the anti-EGFR Fab light chain via a G4S linker.

For expression of the anti-EGFR/anti-CD16 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-EGFR)-CH1-H-CH2-CH3-linker-VL(anti-CD16)-CL (SEQ ID NO:41), encoding the following elements: anti-EGFR heavy chain variable domain followed by human heavy chain constant domains 1-3 from an effector silent IgG1.4 (with mutations as described in Armour et al, Eur J. Immunol. 29:2613, 1999) followed by a G4S linker and anti-CD16 light chain variable domain followed by human kappa light chain constant domain. 2) Construct VL(anti-EGFR)-CL (SEQ ID NO:12), encoding the following elements: anti-EGFR light chain variable domain followed by human kappa light chain constant domain. 3) Construct VH(anti-CD16)-CH1-H (SEQ ID NO:42), encoding the following elements: anti-CD16 heavy chain variable domain followed by human heavy chain constant domain 1 followed by the hinge region EPKSC (SEQ ID NO:10). The corresponding amino acid sequences for these three constructs are shown in SEQ ID NO:43, SEQ ID NO:14, and SEQ ID NO:44, respectively.

For expression of the anti-CD16/anti-EGFR TetBiAb, the following three gene constructs were assembled by standard recombinant DNA techniques and cloned into the mammalian expression vector pTT5, as in FIG. 1: 1) Construct VH(anti-CD16)-CH1-H-CH2-CH3-linker-VL(anti-EGFR)-CL (SEQ ID NO:45), encoding the following elements: anti-CD16 heavy chain variable domain followed by human heavy chain constant domains 1-3 from an effector silent IgG1.4 followed by a G4S linker and anti-EGFR light chain variable domain followed by human kappa light chain constant domain. 2) Construct VL(anti-CD16)-CL (SEQ ID NO:46), encoding the following elements: anti-CD16 light chain variable domain followed by human kappa light chain constant domain. 3) Construct VH(anti-EGFR)-CH1-H (SEQ ID NO:16), encoding the following elements: anti-EGFR heavy chain variable domain followed by human heavy chain constant domain 1 followed by the hinge region EPKSC (SEQ ID NO:10). The corresponding amino acid sequences for these three constructs are shown in SEQ ID NO:47, SEQ ID NO:48, and SEQ ID NO:18, respectively.

Each set of the three vectors was co-transfected transiently into HEK 293-6E cells using Genejuice (Life Technologies, Grand Island, N.Y.) or polyethylenimine (PEI, Polysciences, Warrington, Pa.) for expression of anti-EGFR/anti-CD16 and anti-CD16/anti-EGFR. The two TetBiAbs were purified in a single step by protein A affinity chromatography. Expression of the three polypeptides and assembly of the full hexameric molecule were confirmed on sodium dodecyl sulfate-polyacrylamide gel electrophoresis (SDS-PAGE) and size exclusion chromatography (SEC). For SDS-PAGE, the purified TetBiAbs samples were reduced with DTT and run on NuPAGE MES 4-12% Gel, 200V for 35 min, followed by Coomassie staining.

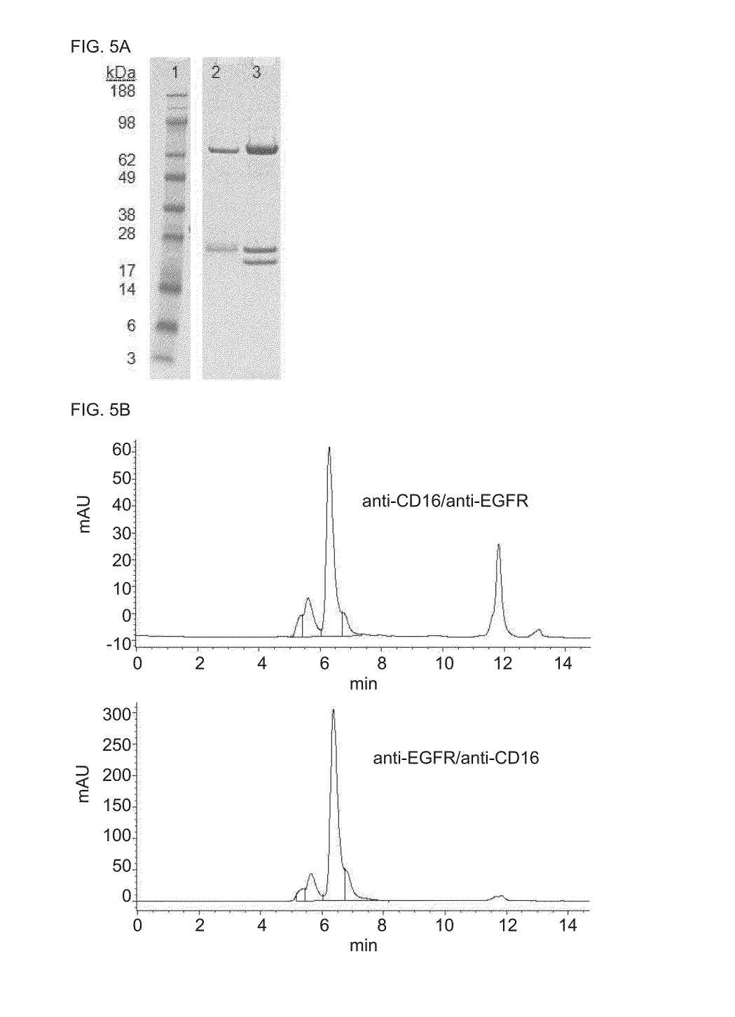

The three major bands on the gel had the expected molecular weights (MW) and the correct stoichiometirc ratio with >95% purity (FIG. 5A). In FIG. 5A, lane 1 shows the molecular weight (MW) marker, lane 2 shows the expected MW (73.6, 23.8, 23.8 kDa) and the correct stoichiometric ratio (1:1:1) of the three polypeptides of anti-CD16/anti-EGFR, and lane 3 shows the expected MW (73.3, 23.6, 23.3 kDa) and the correct stoichiometric ratio (1:1:1) of the three polypeptides of anti-EGFR/anti-CD16. For SEC, the purified TetBiAbs samples were analyzed on a TSK-GEL Super SW3000 SEC column 4.6×300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for both the monomeric anti-EGFR/anti-CD16 and anti-CD16/anti-EGFR (FIG. 5B).

In addition, a number of controls were generated to compare or optimize the TetBiAb format. These include anti-EGFR in a standard monoclonal antibody format (anti-EGFR IgG1) and anti-EGFR in an effector silent format (anti-EGFR IgG1.4).

3B) Binding of TetBiAbs to Antigens

The ability of anti-EGFR/anti-CD16 and anti-CD16/anti-EGFR to retain binding for EGFR was shown by competitive radioligand binding assays. Competing antibodies were mixed with ¹²⁵I-EGF (Perkin Elmer, Waltham, Mass.) prior to the addition of 2 mg of membrane prepared from human A431 epidermoid carcinoma cells that overexpress EGFR. A431 cell membranes were prepared by nitrogen cavitation. The cells were disrupted with 900 psi of with N₂ gas for 30 min, after which the lysate was centrifuged at 1000 g for 10 min at 4° C. The supernatant was collected and centrifuged at 100,000 g for 1 h at 4° C. The resulting pellet was re-suspended with a dounce homogenizer. The protein concentration of the samples was determined using the BioRad protein assay reagent, and the samples were stored frozen at −80° C. for future use. Non-specific binding was determined in the presence of a large excess of unlabeled EGF (100 nM) to saturate all the EGFR binding sites. The reactions were incubated for 90 min at 37° C., with shaking, and terminated by filtering through glass fiber filters (EMD Millipore, Billerica, Mass.). The filters were washed and counted on a gamma counter to determine the amount of ¹²⁵I-EGF bound on the A431 cell membrane.

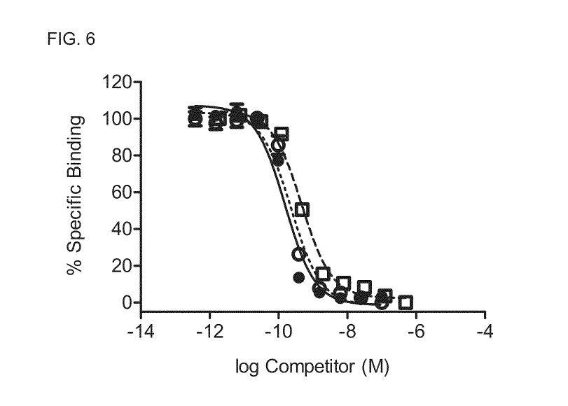

The results show that anti-EGFR/anti-CD16 has a similar ability to inhibit binding of ¹²⁵I-EGF to EGFR on A431 cell membranes as anti-EGFR. Anti-CD16/anti-EGFR also bound to EGFR, although with a slightly higher inhibition constant (Ki) (FIG. 6), showing that the anti-EGFR Fab fused to the C-terminus of another antibody retained binding to EGFR.

3C) Biological Activities of TetBiAbs

The function and utility of anti-EGFR/anti-CD16 and anti-CD16/anti-EGFR were further shown in an antibody-dependent cell-mediated cytotoxicity (ADCC) assay as described in Mueller et al (J. Immunol. 144:1382, 1990). 3×10⁶ human A431 epidermoid carcinoma cells were labeled with 300 μCi of Na ⁵¹Cr (Perkin Elmer, Waltham, Mass.) for 45 min at 37° C. After the cells were washed, 500 cells were transferred to each well of a 96-well plate together with serial dilutions of the recombinant antibodies for concentrations between 0.25-1000 ng/ml. Specific lysis was measured after a 4-hour incubation with effector cells. The effector cells were either resting human peripheral blood mononuclear cells (PBMCs) (effector-to-target cells ratio 100:1) or natural killer (NK) cells (effector-to-target cells ratio 10:1). The NK cells were isolated from the PBMCs with a MACS NK Cell Isolation Kit (Miltenyi Biotec, Bergisch-Gladbach, Germany). Total releasable radioactivity (maximal lysis) was measured by lysing target cells with Triton 100 detergent. Background spontaneous release of radioactivity was measured in wells that contained only target cells. Percentage of specific lysis was calculated by subtracting the background lysis from the experimental values, dividing by the maximal lysis, and multiplying by 100.

Since the Fc of the effector silent IgG1.4 cannot engage the FcγRIII (CD16) on NK cells, this assay requires simultaneous binding of the TetBiAbs for antigens on two different cell types for ADCC to occur. In particular, anti-EGFR/anti-CD16 and anti-CD16/anti-EGFR must engage both EGFR on target A431 cells and CD16 on effector NK cells for killing of and Cr release from the A431 cells to occur.

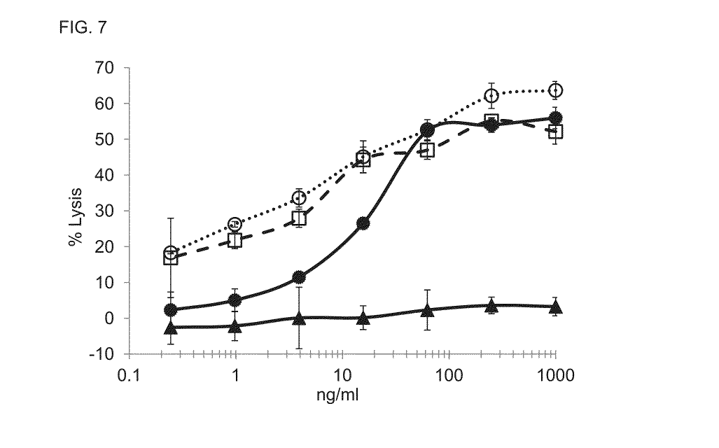

The results show that both anti-EGFR/anti-CD16 and anti-CD16/anti-EGFR with effector silent Fc induced more potent ADCC at low antibody concentrations than the positive control anti-EGFR IgG1 (FIG. 7). This result suggests the effective engagement of FcγRIII by anti-CD16, since the effector silent Fc in both of the TetBiAbs could not contribute to the observed ADCC. Indeed, a negative control anti-EGFR IgG1.4, also comprising the same effector-silent Fc, was unable to induce ADCC (FIG. 7). A therapeutic TetBiAb with the ability to specifically and selectively engage only the FcγRIII is beneficial because to date administration of many therapeutic IgG1 antibodies in the clinic can cause the “first dose effect” of infusion related reactions. These reactions are believed to be due to simultaneous engagement of the Fc to FcγRIII and other activating receptors such as FcγRIIA, leading to cross-linking and systemic activation (McCall et al, J Immunol. 166:6112, 2001).

### Example 4

### Anti-CD20/Anti-CD16

4A) Construction and Expression of TetBiAbs

The generation of the TetBiAbs against CD20 and CD16 is based on the anti-CD20 2B8 (rituximab) monoclonal antibody (Reff et al, Blood 83:435, 1994) and the anti-CD16 3G8 monoclonal antibody (Fleit et al, PNAS 79:3275, 1982 The DNA and protein sequence of the Fab light chain for 2B8 are provided in SEQ ID NO:21 and SEQ ID NO:22, respectively. The DNA and protein sequence of the Fab heavy chain for 2B8 are provided in SEQ ID NO:23 and SEQ ID NO:24, respectively. The DNA and protein sequence of the Fab light chain for 3G8 are provided in SEQ ID NO:37 and SEQ ID NO:38, respectively. The DNA and protein sequence of the Fab heavy chain for 3G8 are provided in SEQ ID NO: 39 and SEQ ID NO:40, respectively. One TetBiAb against CD20 and CD16 molecules was generated: anti-CD20/anti-CD16, in which the C-terminus of the anti-CD20 heavy chain polypeptide is linked to the N-terminus of the anti-CD16 Fab light chain via a G4S linker.

For expression of the anti-CD20/anti-CD16 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-CD20)-CH1-H-CH2-CH3-linker-VL(anti-CD16)-CL (SEQ ID NO:49), encoding the following elements: anti-CD20 heavy chain variable domain followed by human heavy chain constant domains 1-3 isotype IgG1 followed by a G4S linker and anti-CD16 light chain variable domain followed by human kappa light chain constant domain. 2) Construct VL(anti-CD20)-CL (SEQ ID NO:26), encoding the following elements: anti-CD20 light chain variable domain followed by human kappa light chain constant domain. 3) Construct VH(anti-CD16)-CH1-H (SEQ ID NO:50), encoding the following elements: anti-CD16 heavy chain variable domain followed by human heavy chain constant domain 1 followed by the hinge region EPKSC (SEQ ID NO:10). The corresponding amino acid sequences for these three constructs are shown in SEQ ID NO:51, SEQ ID NO:28, and SEQ ID NO:52, respectively.

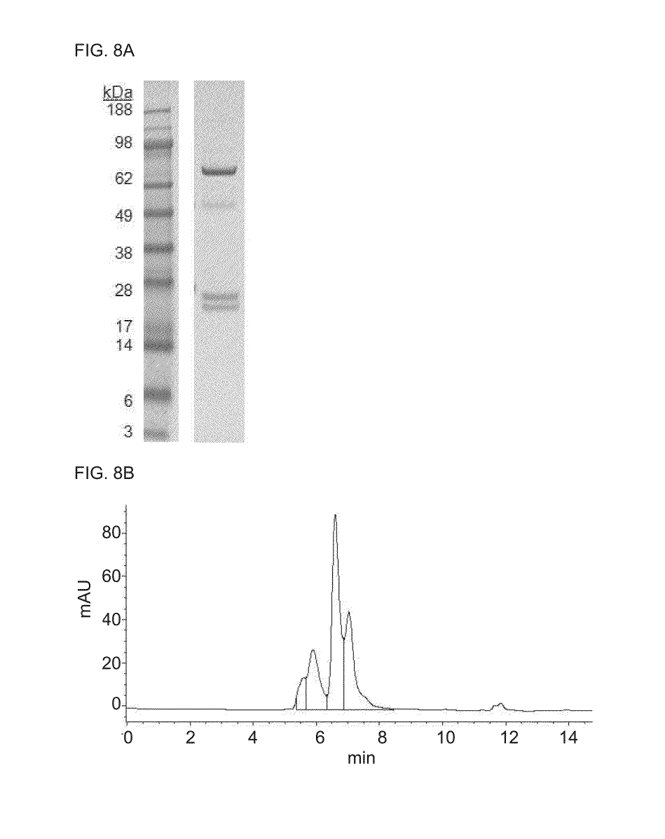

The three vectors were co-transfected transiently into HEK 293-6E cells using Genejuice (Life Technologies, Grand Island, N.Y.) or PEI (Polysciences, Warrington, Pa.) for expression of anti-CD20/anti-CD16. The two TetBiAbs were purified in a single step by protein A affinity chromatography. Expression of the three polypeptides and assembly of the full hexameric molecule were confirmed on SDS-PAGE and SEC. For SDS-PAGE, the purified TetBiAbs samples were reduced with DTT and run on NuPAGE MES 4-12% Gel, 200V for 35 min, followed by Coomassie staining. The three major bands on the gel had the expected MW and the correct stoichiometric ratio with >95% purity (FIG. 8A). In FIG. 8A, lane 1 shows the molecular weight (MW) marker and lane 2 shows the expected MW (73.2, 23.1, 22.9 kDa) and the correct stoichiometric ratio (1:1:1) of the three polypeptides of anti-CD20/anti-CD16. For SEC, the purified TetBiAbs samples were analyzed on a TSK-GEL Super SW3000 SEC column 4.6_{—}300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for the monomeric anti-CD20/anti-CD16 (FIG. 8B).

In addition, anti-CD20 in a standard monoclonal antibody format (anti-CD20 IgG1) was generated as a control to compare with the TetBiAb format.

4B) Binding of TetBiAbs to Antigens

The ability of anti-CD20/anti-CD16 to retain binding to CD20 on the cell surface was measured on human Ramos Burkitt's lymphoma cells, which express CD20 but not CD16. 1×10⁵ Ramos cells per well were incubated with varying concentrations of anti-CD20/anti-CD16 and anti-CD20 diluted in PBS+1% FBS in a 96 well plate for 30 min on ice. After washing with PBS+1% FBS, cells were incubated with TRITC F(ab′)2 goat Anti-Human IgG, Fcγ (Jackson ImmunoResearch, West Grove, Pa.), diluted 1:200 in PBS+1% FBS for 30 min on ice. After washing again, cells were fixed with 1% formaldehyde in PBS. Cells were analyzed by flow cytometry (Guava, EMD Millipore, Billerica, Mass.).

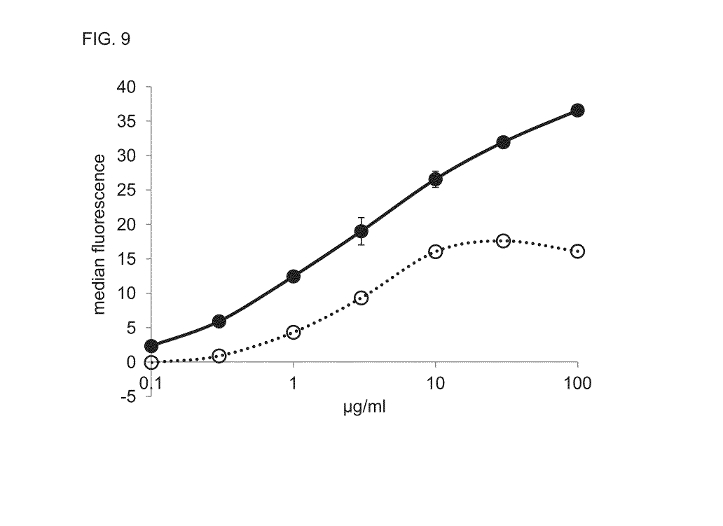

The results show that anti-CD20/anti-CD16 binds to CD20 expressed on Daudi cells (FIG. 9), although not as well as the positive control anti-CD20. While it may be theoretically possible that the anti-CD16 at the C-terminus affected the binding of the anti-CD20 to Daudi cells at the N-terminus, a more likely explanation is a technical one, that the anti-CD16 Fab at the C-terminus affected the accessibility of the Fc to the detecting TRITC F(ab′)2 goat Anti-Human IgG, Fcγ.

4C) Biological Activities of TetBiAbs

The function and utility of anti-CD20/anti-CD16 were further shown by an antibody-dependent cell-mediated cytotoxicity (ADCC) assay using human Ramos Burkitt's lymphoma cells. 2000 cells were transferred to each well of a 96-well plate together with serial dilutions of the recombinant antibodies for concentrations between 0.05-200 ng/ml. Specific lysis was measured via lactate dehydrogenase (LDH) release after a 4-hour incubation with natural killer (NK) effector cells (effector-to-target cells ratio 10:1). The NK cells were isolated from resting human peripheral blood mononuclear cells (PBMCs) with a MACS NK Cell Isolation Kit (Miltenyi Biotec, Bergisch-Gladbach, Germany). Total releasable LDH (maximal lysis) was measured by lysing target cells with Triton 100 detergent. Background spontaneous release of LDH was measured in wells that contained only target cells. Percentage of specific lysis was calculated by subtracting the background lysis from the experimental values, dividing by the maximal lysis, and multiplying by 100.

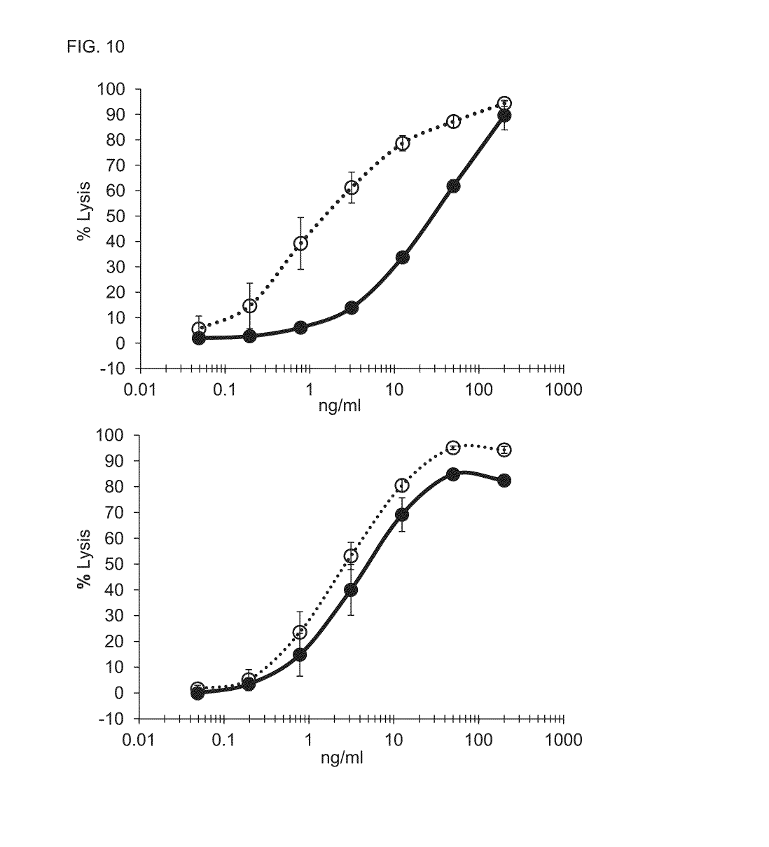

The two graphs of ADCC results show data from different experiments that were executed similarly except using different donors of effector cells. Anti-CD20/anti-CD16, unlike anti-EGFR/anti-CD16, could induce ADCC without engagement of anti-CD16 with CD16 on effectors cells, due to its IgG1 format. However, a ten-fold enhanced induction of ADCC of Ramos cells incubated with anti-CD20/anti-CD16 was observed compared to anti-CD20 with effector cells from four out of seven donors (FIG. 10, upper panel). With the other three donors, the ADCC enhancement of anti-CD20/anti-CD16 over anti-CD20 was marginal (FIG. 10, lower panel). Without being bound by theory, this could possibly be due to the polymorphism in FcRIIIA (Cartron et al, 2002 Blood). It is possible that for donors who are homozygous for the 158-Valine of the FcRIIIA, which has increased binding affinity for the Fc, their FcRIIIA-bearing NK cells would benefit less from the anti-CD16 binding in the ADCC assay, as it appears in FIG. 10, lower panel.

### Example 5

### Anti-CD20/Anti-CD47

5A) Construction and Expression of TetBiAbs

The generation of the TetBiAbs against CD20 and CD47 is based on the anti-CD20 2B8 (rituximab) monoclonal antibody (Reff et al, Blood 83:435, 1994) and the anti-CD47 B6H12 monoclonal antibody (Lindberg et al, JBC 269:1567, 1994). The DNA and protein sequence of the Fab light chain for 2B8 are provided in SEQ ID NO: 21 and SEQ ID NO:22, respectively. The DNA and protein sequence of the Fab heavy chain for 2B8 are provided in SEQ ID NO:23 and SEQ ID NO:24, respectively. The DNA and protein sequence of the Fab light chain for B6H12 are provided in SEQ ID NO: 53 and SEQ ID NO:54, respectively. The DNA and protein sequence of the Fab heavy chain for B6H12 are provided in SEQ ID NO: 55 and SEQ ID NO:56, respectively. One TetBiAb against CD20 and CD47 molecules was generated: anti-CD20/anti-CD47, in which the C-terminus of the anti-CD20 heavy chain polypeptide is linked to the N-terminus of the anti-CD47 Fab light chain via a G4S linker.

For expression of the anti-CD20/anti-CD47 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-CD20)-CH1-H-CH2-CH3-linker-VL(anti-CD47)-CL (SEQ ID NO:57), encoding the following elements: anti-CD20 heavy chain variable domain followed by human heavy chain constant domains 1-3 isotype IgG1 followed by a G4S linker and anti-CD47 light chain variable domain followed by human kappa light chain constant domain. 2) Construct VL(anti-CD20)-CL (SEQ ID NO:26), encoding the following elements: anti-CD20 light chain variable domain followed by human kappa light chain constant domain. 3) Construct VH(anti-CD47)-CH1-H (SEQ ID NO:58), encoding the following elements: anti-CD47 heavy chain variable domain followed by human heavy chain constant domain 1 followed by the hinge region EPKSC (SEQ ID NO:10). The corresponding amino acid sequences for these three constructs are shown in SEQ ID NO:59, SEQ ID NO:28, and SEQ ID NO:60, respectively.

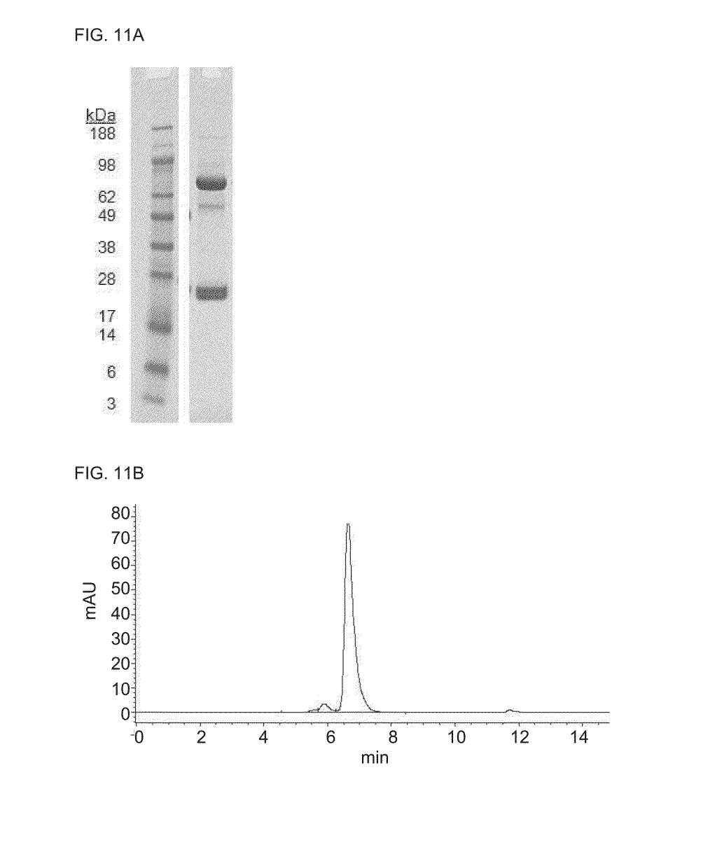

The three vectors were co-transfected transiently into HEK 293-6E cells using Genejuice (Life Technologies, Grand Island, N.Y.) or polyethylenimine (PEI, Polysciences, Warrington, Pa.) for expression of anti-CD20/anti-CD47. The TetBiAb was purified in a single step by protein A affinity chromatography. Expression of the three polypeptides and assembly of the full hexameric molecule were confirmed on SDS-PAGE and SEC. For SDS-PAGE, the purified TetBiAbs samples were reduced with DTT and run on NuPAGE MES 4-12% Gel, 200V for 35 min, followed by Coomassie staining. The three major bands on the gel had the expected MW and the correct stoichiometirc ratio with >95% purity (FIG. 11A). In FIG. 11A, lane 1 shows the molecular weight (MW) marker and lane 2 shows the expected MW (73.8, 23.4, 23.0 kDa) and the correct stoichiometric ratio (1:1:1) of the three polypeptides of anti-CD20/anti-CD47. For SEC, the purified TetBiAbs samples were analyzed on a TSK-GEL Super SW3000 SEC column 4.6×300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for the monomeric anti-CD20/anti-CD47 (FIG. 11B).

In addition, anti-CD20 and anti-CD47 in a standard monoclonal antibody format (anti-CD20 IgG1 and anti-CD47 IgG1) were generated as controls to compare with the TetBiAb format.

5B Binding of TetBiAbs

(i) Binding of TetBiAbs to antigens The ability of anti-CD20/anti-CD47 to bind to both antigens expressed on the cell surface was measured, and compared to the two control molecules anti-CD20 and anti-CD47. 1×10⁵ mouse NS0 myeloma cells transfected with CD20 or human U937 histiocytic lymphoma cells per well were incubated with varying concentrations of antibodies diluted in PBS+1% FBS in a 96 well plate for 30 min on ice. After washing with PBS+1% FBS, cells were incubated with TRITC F(ab′)2 goat Anti-Human IgG, Fcγ (Jackson ImmunoResearch, West Grove, Pa.) diluted 1:200 in PBS+1% FBS for 30 min on ice. After washing again, cells were fixed with 1% formaldehyde in PBS. Cells were analyzed by flow cytometry (Guava, EMD Millipore, Billerica, Mass.).

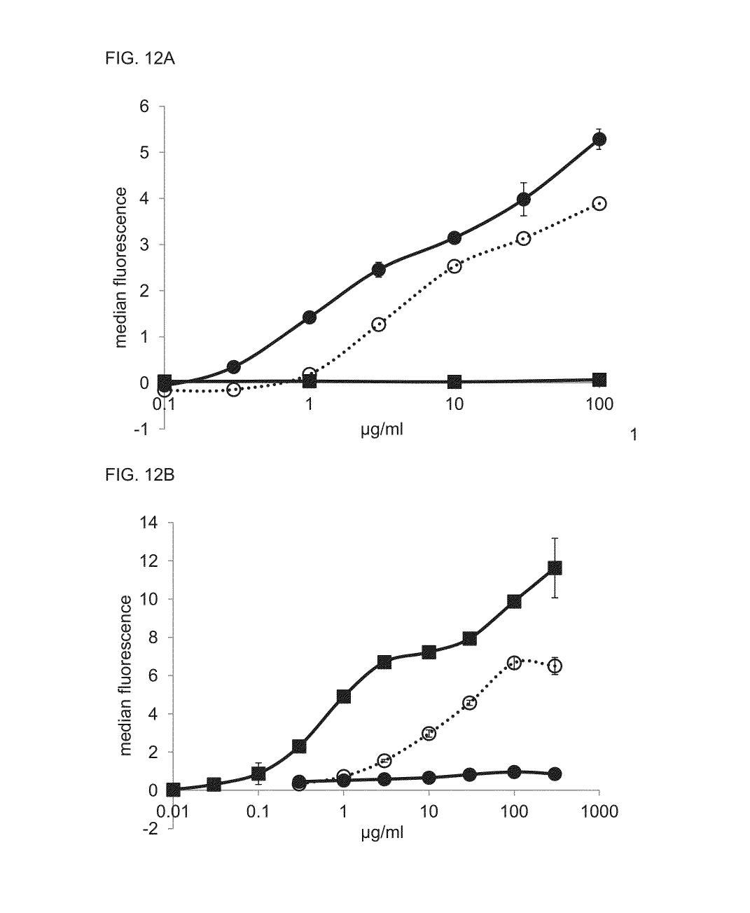

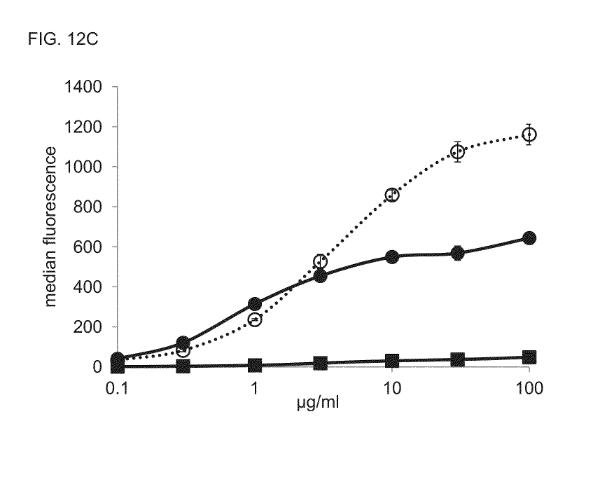

The results show that anti-CD20/anti-CD47 and anti-CD20 bind to CD20 expressed on CD20-tranfected NS0 cells, but anti-CD47 does not bind to NS0/CD20 cells because CD47 is not expressed (FIG. 12A). Similar to the case of the previously observed case of apparently decreased binding of anti-CD20/anti-CD16 to CD20 in Example 4B, it is unlikely that the anti-CD47 at the C-terminus affected the binding of the anti-CD20 at the N-terminus to CD20 (FIG. 12A). A more likely explanation is that the anti-CD47 Fab at the C-terminus affected the accessibility of the Fc to the detecting TRITC F(ab′)2 goat Anti-Human IgG, Fcγ, thereby resulting in the observed apparent decreased binding in FIG. 12A. Anti-CD20/anti-CD47 and anti-CD47 bind to U937 cells and anti-CD20 does not bind to U937 cells, which express CD47 but not CD20 (FIG. 12B). The binding of anti-CD20/anti-CD47 to U937 cells shows that anti-CD47 as C-terminal Fab, attached to the C-terminus of Fc by means of the light chain, can still recognize its antigen (FIG. 12B). The slight decrease in binding observed is similar to the decrease observed for anti-EGFR Fab when attached to the C-terminus of Fc (FIGS. 2 and 3).

(ii) Binding avidity of anti-CD20/anti-CD47 TetBiAb on cells expressing both antigens.

Binding of anti-CD20/anti-CD47 to CD20 and CD47 on the cell surface was measured on human SU-DHL4 B cell lymphoma cells that overexpress CD20 and express CD47 at low levels. Anti-CD20/anti-CD47, anti-CD20, and anti-CD47 were conjugated with Alexa Fluor® 488 carboxylic acid, TFP ester, bis (triethylammonium salt) (Life Technologies, Grand Island, N.Y.). 1×10⁵ SU-DHL4 cells per well were incubated with varying concentrations of Alexa 488-labeled anti-CD20/anti-CD47, anti-CD20, and anti-CD47 diluted in PBS+1% FBS in a 96 well plate for 60 min on ice. After washing with PBS+1% FBS, cells were fixed with 1% formaldehyde in PBS. Cells were analyzed by flow cytometry (MACSQuant, Miltenyi Biotec, Cologne, Germany).

The results show that anti-CD20/anti-CD47 binding to SU-DHL4 cells is enhanced compared to the binding of anti-CD20 or of anti-CD47, either individually or in combination, to SU-DHL4 cells (FIG. 12C), providing strong evidence for avidity. This is especially striking because of the low level of CD47 expression on SU-DHL4 cells, as evidenced by the low median florescence obtained binding of anti-CD47. The ability of a TetBiAb to harness the avidity of binding to the tumor cells by binding to two tumor targets on the same cell may result in more specific targeting and less side effects in vivo.

5C) Biological Activities of TetBiAbs

The utility of anti-CD20/anti-CD47 is shown by an in vivo experiment. In a disseminated lymphoma model, SCID mice are injected i.v. with 5×10⁶ CD20+ human Raji lymphoma cells, followed by i.v. injection of 200 mg/mouse of an antibody isotype control (Group 1), 200 mg/mouse of anti-CD20 (Group 2), 200 mg/mouse of anti-CD47 (Group 3), combination of 200 mg/mouse of anti-CD20 and 200 mg/mouse of anti-CD47 (Group 4), or 333 mg/mouse of anti-CD20/anti-CD47, which is the equimolar amount of tetravalent bispecific antibody (Group 5). All the groups (n=10) receive weekly injections and results are reported as general health, e.g. paralysis, which precedes death by 10-14 days, and survival of mice. Treatment with anti-CD20/anti-CD47 tetravalent bispecific antibody (Group 5) is found to be at least as efficacious as the combination therapy (Group 4), but superior to the two monotherapies (groups 2 and 3).

### Example 6

### Anti-CD20/Anti-CD52 and Anti-CD52/Anti-CD20

6A) Construction and Expression of TetBiAbs

The generation of the TetBiAbs against CD20 and CD52 is based on the anti-CD20 2B8 (rituximab) monoclonal antibody (Reff et al, Blood 83:435, 1994) and the anti-CD52 Campath monoclonal antibody (James et al, JMB 289:293, 1999). The DNA and protein sequence of the Fab light chain for 2B8 are provided in SEQ ID NO:21 and SEQ ID NO:22, respectively. The DNA and protein sequence of the Fab heavy chain for 2B8 are provided in SEQ ID NO:23 and SEQ ID NO:24, respectively. The DNA and protein sequence of the Fab light chain for Campath are provided in SEQ ID NO:61 and SEQ ID NO:62, respectively. The DNA and protein sequence of the Fab heavy chain for Campath are provided in SEQ ID NO: 63 and SEQ ID NO:64, respectively. Two different TetBiAbs against CD20 and CD52 molecules were generated: (i) anti-CD20/anti-CD52, in which the C-terminus of the anti-CD20 heavy chain polypeptide is linked to the N-terminus of the anti-CD52 Fab light chain via a G4S linker and (ii) anti-CD52/anti-CD20, in which the C-terminus of the anti-CD52 heavy chain polypeptide is linked to the N-terminus of the anti-CD20 Fab light chain via a G4S linker.

For expression of the anti-CD20/anti-CD52 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-CD20)-CH1-H-CH2-CH3-linker-VL(anti-CD52)-CL (SEQ ID NO:65), encoding the following elements: anti-CD20 heavy chain variable domain followed by human heavy chain constant domains 1-3 isotype IgG1 followed by a G4S linker and anti-CD52 light chain variable domain followed by human kappa light chain constant domain. 2) Construct VL(anti-CD20)-CL (SEQ ID NO:26), encoding the following elements: anti-CD20 light chain variable domain followed by human kappa light chain constant domain. 3) Construct VH(anti-CD52)-CH1-H (SEQ ID NO:66), encoding the following elements: anti-CD52 heavy chain variable domain followed by human heavy chain constant domain 1 followed by the hinge region EPKSC (SEQ ID NO:10). The corresponding amino acid sequences for these three constructs are shown in SEQ ID NO:67, SEQ ID NO:28, and SEQ ID NO:68, respectively.

For expression of the anti-CD52/anti-CD20 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-CD52)-CH1-H-CH2-CH3-linker-VL(anti-CD20)-CL (SEQ ID NO:69), encoding the following elements: anti-CD52 heavy chain variable domain followed by human heavy chain constant domains 1-3 isotype IgG1 followed by a G4S linker and anti-CD20 light chain variable domain followed by human kappa light chain constant domain. 2) Construct VL(anti-CD52)-CL (SEQ ID NO:70), encoding the following elements: anti-CD52 light chain variable domain followed by human kappa light chain constant domain. 3) Construct VH(anti-CD20)-CH1-H (SEQ ID NO:32), encoding the following elements: anti-CD20 heavy chain variable domain followed by human heavy chain constant domain 1 followed by the hinge region EPKSC (SEQ ID NO:10). The corresponding amino acid sequences for these three constructs are shown in SEQ ID NO:71, SEQ ID NO:72, and SEQ ID NO:34, respectively.

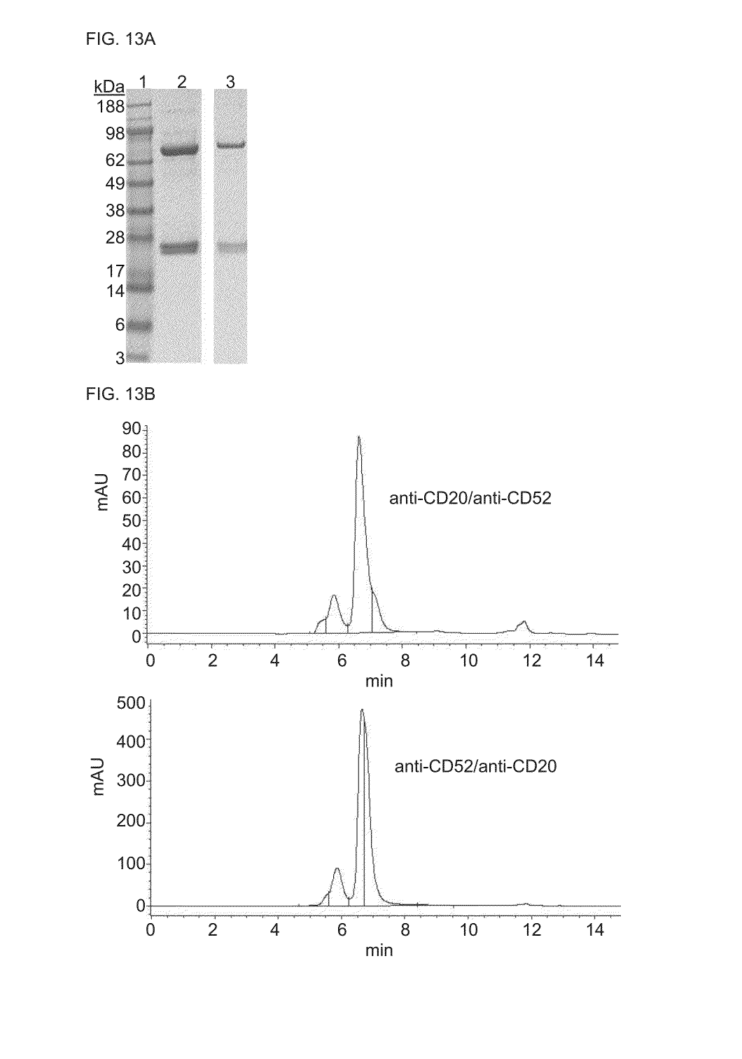

Each set of the three vectors was co-transfected transiently into HEK 293-6E cells using Genejuice (Life Technologies, Grand Island, N.Y.) or polyethylenimine (PEI, Polysciences, Warrington, Pa.) for expression of anti-CD20/anti-CD52 and anti-CD52/anti-CD20. The two TetBiAbs were purified in a single step by protein A affinity chromatography. Expression of the three polypeptides and assembly of the full hexameric molecule were confirmed on sodium dodecyl sulfate-polyacrylamide gel electrophoresis (SDS-PAGE) and size exclusion chromatography (SEC). For SDS-PAGE, the purified TetBiAbs samples were reduced with DTT and run on NuPAGE MES 4-12% Gel, 200V for 35 min, followed by Coomassie staining. The three major bands on the gel had the expected molecular weights (MW) and the correct stoichiometirc ratio with >95% purity (FIG. 13A). In FIG. 13A, lane 1 shows the molecular weight (MW) marker, lane 2 shows the expected MW (73.0, 23.4, 23.1 kDa) and the correct stoichiometric ratio (1:1:1) of the three polypeptides of anti-CD20/anti-CD52, and lane 3 shows the expected MW (72.7, 23.5, 23.2 kDa) and the correct stoichiometric ratio (1:1:1) of the three polypeptides of anti-CD52/anti-CD20. For SEC, the purified TetBiAbs samples were analyzed on a TSK-GEL Super SW3000 SEC column 4.6_{—}300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for both the monomeric anti-CD20/anti-CD52 and anti-CD52/anti-CD20 (FIG. 13B).

In addition, a number of controls were generated to compare or optimize the TetBiAb format. These include anti-CD20 and anti-CD52 in standard monoclonal antibody format (anti-CD20 IgG1 and anti-CD52 IgG1).

6B) Binding of TetBiAbs to Antigens

The ability of anti-CD20/anti-CD52 and anti-CD52/anti-CD20 to bind both CD20 and CD52 expressed on the cell surface was measured, and compared to the two control molecules anti-CD20 and anti-CD52. 1×10⁵ human Daudi Burkitt's lymphoma cells or human Kasumi-3 acute myeloblastic leukemia cells per well were incubated with varying concentrations of antibodies diluted in PBS+1% FBS in a 96 well plate for 30 min on ice. After washing with PBS+1% FBS, cells were incubated with TRITC F(ab′)2 goat Anti-Human IgG, Fcγ (Jackson ImmunoResearch, West Grove, Pa.) diluted 1:200 in PBS+1% FBS for 30 min on ice. After washing again, cells were fixed with 1% formaldehyde in PBS. Cells were analyzed by flow cytometry (Guava, EMD Millipore, Billerica, Mass.).

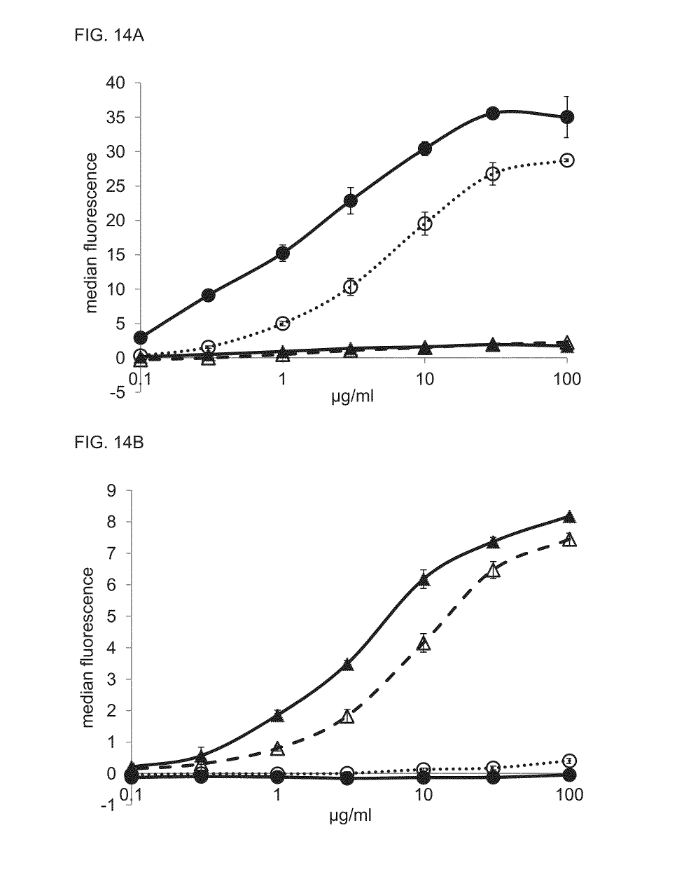

The results show that anti-CD20/anti-CD52 and anti-CD20 bind to Daudi cells, and anti-CD52/anti-CD20 and anti-CD52 do not bind to Daudi cells, which express CD20 but not CD52 (FIG. 14A). Moreover, anti-CD52/anti-CD20 and anti-CD52 bind to Kasumi-3 cells, and anti-CD20/anti-CD52 and anti-CD20 do not bind to Kasumi-3 cells, which express CD52 but not CD20 (FIG. 14B).

These results are in agreement with the results from FIG. 4 that show that anti-CD20 Fab does not bind CD20 expressed on cell membrane when it is attached at the C-terminus of the Fc region via a Fab light chain. Both CD52 and CD20 are transmembrane proteins containing small extra-cellular domains, and anti-CD20 and anti-CD52 only bind to an extracellular loop. It is likely that the Fc region hinders accessibility of the C-terminal Fab to bind the small loops. By way of example, this combination of targets may be a good candidate for a TetBiAb in the alternate conformation illustrated in FIG. 1C and FIG. 1D. In example 2, it was shown that anti-CD20 Fab retained binding when attached to the C-terminus of Fc region via a Fab heavy chain, but did not when attached via a light chain. Thus, an anti-CD20 Fab is attached to the C-terminus of the Fc region via a Fab heavy chain, and an anti-CD52 Fab is attached to the N-terminus of the Fc region via a light chain (rather than via CH1 as in the standard monoclonal antibody format). The binding of the resulting anti-CD52/anti-CD20 to both antigens is then tested. A further variation is engineered and tested as well, with anti-CD20 Fab attached to the N-terminus of the Fc region via the light chain (Schaefer et al. Proc Natl Acad Sci USA. 108:11187, 2011) and anti-CD52 Fab attached to the C-terminus of Fc region via the Fab heavy chain.

### Example 7

### Fc-Anti-CD47 Precursor Molecules

7A) Construction and Expression of Fc-Fab Precursors

The generation of the Fc-anti-CD47 is based on the anti-CD47 B6H12 monoclonal antibody (Lindberg et al, JBC 269: 1567, 1994). The DNA and protein sequence of the Fab light chain for B6H12 are provided in SEQ ID NO:53 and SEQ ID NO:54, respectively. The DNA and protein sequence of the Fab heavy chain for B6H12 are provided in SEQ ID NO:55 and SEQ ID NO:56, respectively. Two different Fc-CD47 molecules were generated: (i) Fc-(G4S)₄-anti-CD47(VHCH1), in which the C-terminus of the Fc heavy heavy chain is linked to the N-terminus of the anti-CD47 Fab heavy chain via a (G4S)₄ linker and (ii) Fc-(G4S)₄-anti-CD47(LC), in which the C-terminus of the Fc region heavy chain is linked to the N-terminus of the anti-CD47 Fab light chain via a (G4S)₄ linker.

For expression of Fc-(G4S)₄-anti-CD47(VHCH1), the following two gene constructs were assembled by standard recombinant DNA techniques and cloned into the mammalian expression vector pTT5 (containing the mouse light chain signal peptide sequence for secretion): 1) Construct H-CH2-CH3-(G4S)₄-VH(anti-CD47)-CH1-H (SEQ ID NO:73), encoding the following elements: a human heavy chain hinge region with cysteine (which natively forms a disulfide bond with the light chain) mutated to a serine, (EPKSS, SEQ ID NO:8), followed by constant domains 2 and 3, followed by a (G4S)₄ linker, and anti-CD47 heavy chain variable domain followed by human heavy chain constant domain 1 followed by the hinge region (EPKSC, SEQ ID NO:10, to allow for a disulfide bridge with the anti-CD47 light chain); and 2) Construct VL(anti-CD47)-CL (SEQ ID NO:74), encoding the following elements: an anti-CD47 light chain variable domain followed by human kappa light chain constant domain. The corresponding amino acid sequences for these two constructs are shown in SEQ ID NO:75 and SEQ ID NO:76 respectively.

For expression of Fc-(G4S)₄-anti-CD47(LC), the following two gene constructs were assembled by standard recombinant DNA techniques and cloned into the mammalian expression vector pTT5 (containing the mouse light chain signal peptide sequence for secretion): 1) Construct H-CH2-CH3-(G4S)₄-VL(anti-CD47)-CL (SEQ ID NO:77), encoding the following elements: a human heavy chain hinge region EPKSC (SEQ ID NO:8) followed by constant domains 2 and 3, followed by a (G4S)₄ linker, and anti-CD47 light chain variable domain followed by human kappa light chain constant domain; and 2) Construct VH(anti-CD47)-CH1-H (SEQ ID NO:58), encoding the following elements: anti-CD47 heavy chain variable domain followed by human heavy chain constant domain 1 followed by the hinge region EPKSC (SEQ ID NO:10). The corresponding amino acid sequences for these two constructs are shown in SEQ ID NO:78 and SEQ ID NO:60, respectively.

Each set of the two vectors was co-transfected transiently into HEK 293-6E cells using Genejuice (Life Technologies, Grand Island, N.Y.) or polyethylenimine (PEI, Polysciences, Warrington, Pa.) for expression of Fc-(G4S)₄-anti-CD47(VHCH1) and Fc-(G4S)₄-anti-CD47(LC). The proteins were purified in a single step by protein A affinity chromatography. Expression of the two polypeptides and assembly of the full tetrameric molecule were confirmed on sodium dodecyl sulfate-polyacrylamide gel electrophoresis (SDS-PAGE) and size exclusion chromatography (SEC).

In addition, a control anti-CD47 in a standard monoclonal antibody format (anti-CD47 IgG1) was generated to compare to the different Fc-Fab formats.

7B) Binding of Fc-Fab Precursors to Antigens

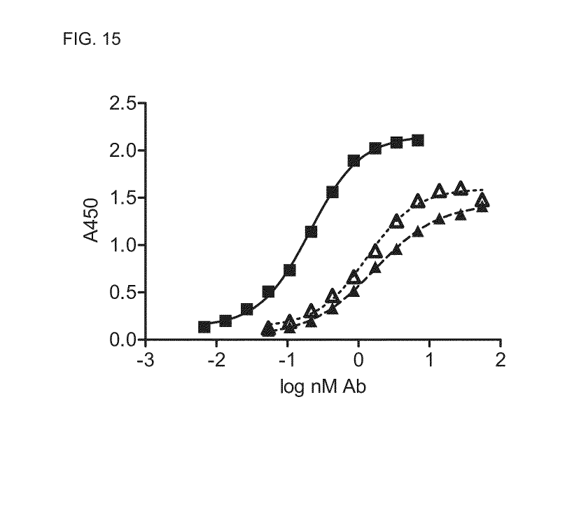

The ability of Fc-(G4S)₄-anti-CD47(VHCH1) and Fc-(G4S)₄-anti-CD47(LC) to bind to CD47 was measured via ELISA, and compared to the control molecules anti-CD47. Human CD47 was coated on 96 well plates overnight at 4° C. After washing with PBST, the wells were blocked with PBST+2% BSA for 1 hr at room temperature. After washing with PBST, varying concentrations of antibodies diluted in PBST+2% BSA were added to the wells and incubated for 1 hr at room temperature. After washing with PBST, HRP-conjugated Goat anti-Human IgG Fcγ (Jackson ImmunoResearch, West Grove, Pa.) diluted 1:10000 in PBST+2% BSA was added to the wells and incubated for 1 hr at room temperature. The bound antibodies were visualized with HRP substrate, 3,3′,5,5′-tetramethylbenzidine (TMB). The plates were measured for absorbance at 450 nm. The results show that Fc-(G4S)₄-anti-CD47(VHCH1) and Fc-(G4S)₄-anti-CD47(LC) retain binding to CD47, although not as well as anti-CD47 IgG1 (FIG. 15).

### Example 8

### Anti-EGFR/Anti-CD47 and Anti-CD47/Anti-EGFR

8A) Construction and Expression of TetBiAbs

The generation of the TetBiAbs against EGFR and CD47 is based on the anti-EGFR C225 (cetuximab) monoclonal antibody (Kawamoto, PNAS 80:1337, 1983) and the anti-CD47 B6H12 monoclonal antibody (Lindberg et al, JBC 269: 1567, 1994). The DNA and protein sequence of the Fab light chain for C225 are provided in SEQ ID NO:1 and SEQ ID NO:2, respectively. The DNA and protein sequence of the Fab heavy chain for C225 are provided in SEQ ID NO:3 and SEQ ID NO:4, respectively. The DNA and protein sequence of the Fab light chain for B6H12 are provided in SEQ ID NO:53 and SEQ ID NO:54, respectively. The DNA and protein sequence of the Fab heavy chain for B6H12 are provided in SEQ ID NO:55 and SEQ ID NO:56, respectively. Two different TetBiAbs against EGFR and CD47 molecules were generated: (i) anti-EGFR/anti-CD47, in which the C-terminus of the anti-EGFR heavy chain polypeptide is linked to the N-terminus of the anti-CD47 Fab light chain via a (G4S)₄ linker and (ii) anti-CD47/anti-EGFR, in which the C-terminus of the anti-CD47 heavy chain polypeptide is linked to the N-terminus of the anti-EGFR Fab light chain via a (G4S)₄ linker.

For expression of the anti-EGFR/anti-CD47 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-EGFR)-CH1-H-CH2-CH3-(G4S)₄-VL(anti-CD47)-CL (SEQ ID NO:79), encoding the following elements: anti-EGFR heavy chain variable domain followed by human heavy chain constant domains 1-3 followed by a (G4S)₄ linker and anti-CD47 light chain variable domain followed by human kappa light chain constant domain. 2) Construct VL(anti-EGFR)-CL (SEQ ID NO:12), encoding the following elements: anti-EGFR light chain variable domain followed by human kappa light chain constant domain. 3) Construct VH(anti-CD47)-CH1-H (SEQ ID NO:58), encoding the following elements: anti-CD47 heavy chain variable domain followed by human heavy chain constant domain 1 followed by the hinge region EPKSC (SEQ ID NO:10). The corresponding amino acid SEQ ID NO:for these three constructs are shown in SEQ ID NO:80, SEQ ID NO:14, and SEQ ID NO:60 respectively.

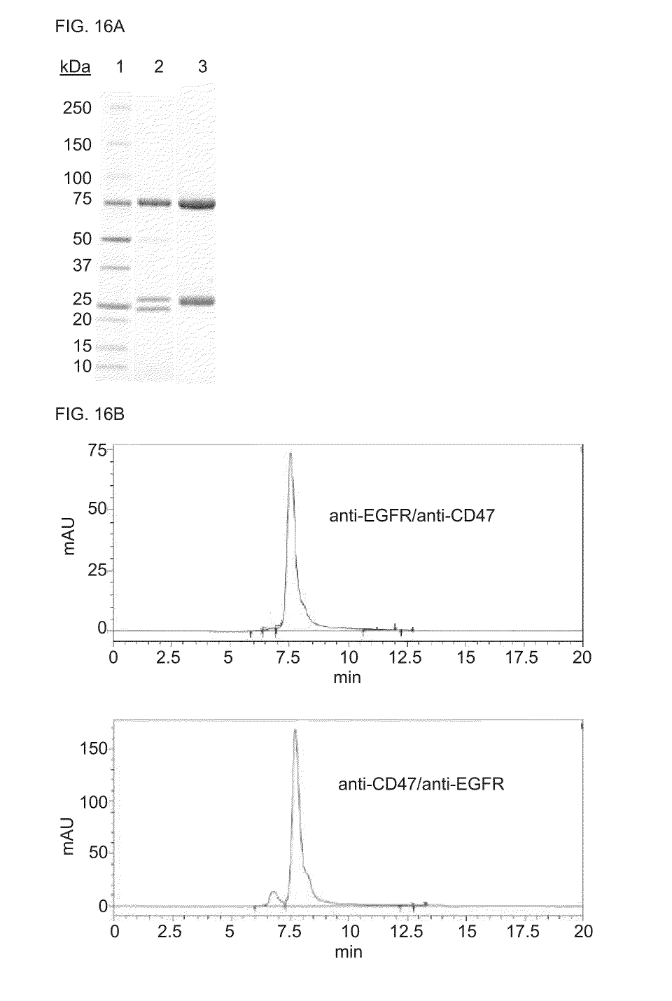

For expression of the anti-CD47/anti-EGFR TetBiAb, the following three gene constructs were assembled by standard recombinant DNA techniques and cloned into the mammalian expression vector pTT5, as in FIG. 1: 1) Construct VH(anti-CD47)-CH1-H-CH2-CH3-(G4S)₄-VL(anti-EGFR)-CL (SEQ ID NO:81), encoding the following elements: anti-CD47 heavy chain variable domain followed by human heavy chain constant domains 1-3 followed by a (G4S)₄ linker and anti-EGFR light chain variable domain followed by human kappa light chain constant domain. 2) Construct VL(anti-CD47)-CL (SEQ ID NO:74), encoding the following elements: anti-CD47 light chain variable domain followed by human kappa light chain constant domain. 3) Construct VH(anti-EGFR)-CH1-H (SEQ ID NO:16), encoding the following elements: anti-EGFR heavy chain variable domain followed by human heavy chain constant domain 1 followed by the hinge region EPKSC (SEQ ID NO:10). The corresponding amino acid SEQ ID NO:for these three constructs are shown in SEQ ID NO:82, SEQ ID NO:76, and SEQ ID NO:18 respectively. Each set of the three vectors was co-transfected transiently into Expi293 cells using Expi293fectin (Life Technologies, Grand Island, N.Y.) for expression of anti-EGFR/anti-CD47 and anti-CD47/anti-EGFR. The two TetBiAbs were purified in a single step by protein A affinity chromatography. Expression of the three polypeptides and assembly of the full hexameric molecule were confirmed on sodium dodecyl sulfate-polyacrylamide gel electrophoresis (SDS-PAGE) and size exclusion chromatography (SEC). For SDS-PAGE, the purified TetBiAbs samples were reduced with DTT and run on NuPAGE MES 4-12% Gel, 200V for 35 min, followed by Coomassie staining. The three major bands on the gel had the expected molecular weights (MW) and the correct stoichiometric ratio with >95% purity (FIG. 16A). In FIG. 16A, lane 1 shows the molecular weight (MW) marker, lane 2 shows the expected MW (74, 24, 23 kDa) and the correct stoichiometric ratio (1:1:1) of the three polypeptides of anti-CD47/anti-EGFR, and lane 3 shows the expected MW (74, 23, 23 kDa) and the correct stoichiometric ratio (1:1:1) of the three polypeptides of anti-EGFR/anti-CD47. For SEC, the purified TetBiAbs samples were analyzed on a TSK-GEL Super SW3000 SEC column 4.6_{—}300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for both the monomeric anti-EGFR/anti-CD47 and anti-CD47/anti-EGFR (FIG. 16B).

In addition, a number of controls were generated to compare or optimize the TetBiAb format. These include anti-EGFR in a standard monoclonal antibody format (anti-EGFR IgG1) and anti-CD47 in a standard monoclonal antibody format (anti-CD47 IgG1).

8B) Binding of TetBiAbs

(i) Binding of TetBiAbs to Antigens

The ability of anti-EGFR/anti-CD47 and anti-CD47/anti-EGFR to retain binding to CD47 was measured by ELISA. Human CD47 was coated on 96 well plates overnight at 4° C. After washing with PBST, the wells were blocked with PBST+2% BSA for 1 hr at room temperature. After washing with PBST, varying concentrations of antibodies diluted in PBST+2% BSA were added to the wells and incubated for 1 hr at room temperature. After washing with PBST, HRP-conjugated Goat anti-Human IgG Fcγ (Jackson ImmunoResearch, West Grove, Pa.) diluted 1:10000 in PBST+2% BSA was added to the wells and incubated for 1 hr at room temperature. The bound antibodies were visualized with HRP substrate, 3,3′,5,5′-tetramethylbenzidine (TMB). The plates were measured for absorbance at 450 nm.

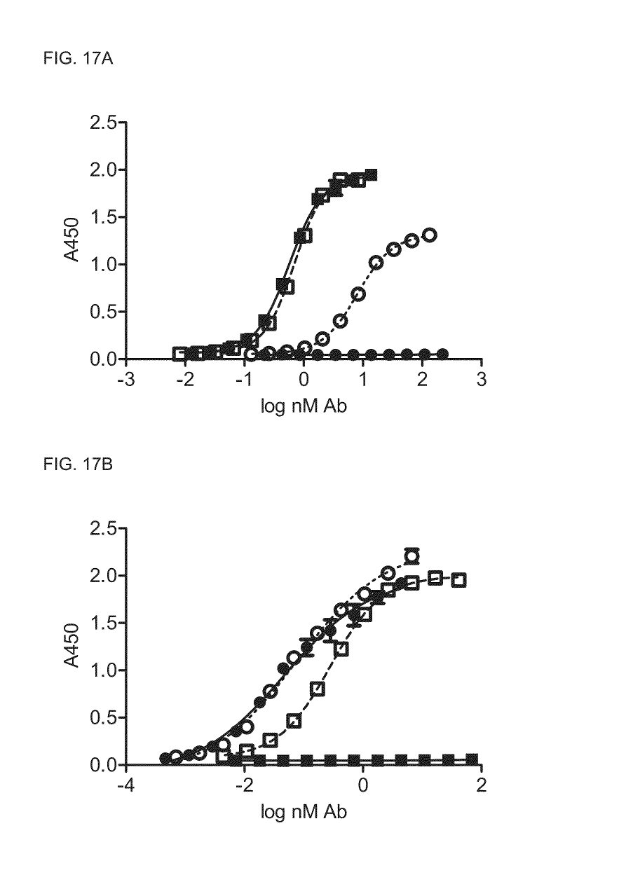

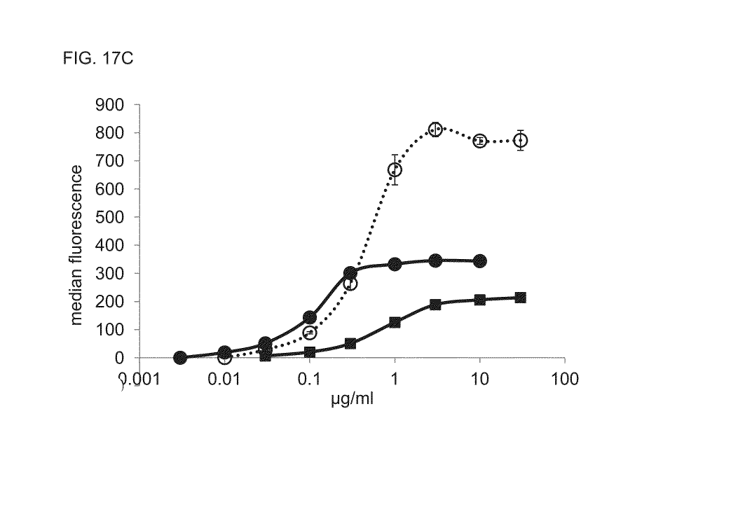

The results show that anti-CD47/anti-EGFR retains binding to CD47, similar to anti-CD47. Anti-EGFR/anti-CD47 also retains binding to CD47, although it does not bind as well as anti-CD47 (FIG. 17A).

The ability of anti-EGFR/anti-CD47 and anti-CD47/anti-EGFR to retain binding to EGFR was measured by ELISA. Human EGFR was coated on 96 well plates overnight at 4° C. After washing with PBST, the wells were blocked with PBST+2% BSA for 1 hr at room temperature. After washing with PBST, varying concentrations of antibodies diluted in PBST+2% BSA were added to the wells and incubated for 1 hr at room temperature. After washing with PBST, HRP-conjugated Goat anti-Human IgG Fcγ (Jackson ImmunoResearch, West Grove, Pa.) diluted 1:10000 in PBST+2% BSA was added to the wells and incubated for 1 hr at room temperature. The bound antibodies were visualized with HRP substrate, 3,3′,5,5′-tetramethylbenzidine (TMB). The plates were measured for absorbance at 450 nm.

The results show that anti-EGFR/anti-CD47 retains binding to EGFR, similar to anti-EGFR. Anti-CD47/anti-EGFR also retains binding to EGFR, although it does not bind as well as anti-EGFR (FIG. 17B).

(ii) Binding Avidity of Anti-EGFR/Anti-CD47 TetBiAb on Cells Expressing Both Antigens.

The ability of anti-EGFR/anti-CD47 to bind with avidity to EGFR and CD47 on the cell surface was measured on human A431 epidermoid carcinoma cells that overexpress EGFR and express CD47. anti-EGFR/anti-CD47, anti-EGFR, and anti-CD47 were conjugated with Alexa Fluor® 488 carboxylic acid, TFP ester, bis (triethylammonium salt) (Life Technologies, Grand Island, N.Y.). 1×10⁵ A431 cells per well were incubated with varying concentrations of Alexa 488-labeled anti-EGFR/anti-CD47, anti-EGFR, and anti-CD47 diluted in PBS+1% FBS in a 96 well plate for 60 min on ice. After washing with PBS+1% FBS, cells were fixed with 1% formaldehyde in PBS. Cells were analyzed by flow cytometry (MACSQuant, Miltenyi Biotec, Cologne, Germany).

The results show that anti-EGFR/anti-CD47 binding to A431 cells is enhanced compared to the binding of anti-EGFR or anti-CD47, individually or in combination, to A431 cells (FIG. 17C), providing strong evidence for avidity. The ability of a TetBiAb to harness the avidity of binding to the tumor cells by binding to two tumor targets on the same cell may result in more specific targeting and less side effects in vivo.

### Example 9

### Anti-HER2/Anti-CD47 and Anti-CD47/Anti-HER2

9A) Construction and Expression of TetBiAbs

The generation of the TetBiAbs against HER2 and CD47 is based on the anti-HER2 4D5 (trastuzumab) monoclonal antibody (Carter et al, PNAS 89: 4285, 1992) and the anti-CD47 B6H12 monoclonal antibody (Lindberg et al, JBC 269: 1567, 1994). The DNA and protein sequence of the Fab light chain for 4D5 are provided in SEQ ID NO:83 and SEQ ID NO:84, respectively. The DNA and protein sequence of the Fab heavy chain for 4D5 are provided in SEQ ID NO:85 and SEQ ID NO:86, respectively. The DNA and protein sequence of the Fab light chain for B6H12 are provided in SEQ ID NO:53 and SEQ ID NO:54, respectively. The DNA and protein sequence of the Fab heavy chain for B6H12 are provided in SEQ ID NO:55 and SEQ ID NO:56, respectively. Two different TetBiAbs against HER2 and CD47 molecules were generated: (i) anti-HER2/anti-CD47, in which the C-terminus of the anti-HER2 heavy chain polypeptide is linked to the N-terminus of the anti-CD47 Fab light chain via a (G4S)₄ linker and (ii) anti-CD47/anti-HER2, in which the C-terminus of the anti-CD47 heavy chain polypeptide is linked to the N-terminus of the anti-HER2 Fab light chain via a (G4S)₄ linker.

For expression of the anti-HER2/anti-CD47 TetBiAb, the following three gene constructs were assembled by standard recombinant DNA techniques and cloned into the mammalian expression vector pTT5 (containing the mouse light chain signal peptide sequence for secretion), as in FIG. 1: 1) Construct VH(anti-HER2)-CH1-H-CH2-CH3-(G4S)₄-VL(anti-CD47)-CL (SEQ ID NO:87), encoding the following elements: anti-HER2 heavy chain variable domain followed by human heavy chain constant domains 1-3 followed by a (G4S)₄ linker and anti-CD47 light chain variable domain followed by human kappa light chain constant domain. 2) Construct VL(anti-HER2)-CL (SEQ ID NO:88), encoding the following elements: anti-HER2 light chain variable domain followed by human kappa light chain constant domain. 3) Construct VH(anti-CD47)-CH1-H (SEQ ID NO:58), encoding the following elements: anti-CD47 heavy chain variable domain followed by human heavy chain constant domain 1 followed by the hinge region EPKSC (SEQ ID NO:10). The corresponding amino acid sequences for these three constructs are shown in SEQ ID NO:89, SEQ ID NO:90, and SEQ ID NO:60 respectively.

For expression of the anti-CD47/anti-HER2 TetBiAb, the following three gene constructs were assembled by standard recombinant DNA techniques and cloned into the mammalian expression vector pTT5, as in FIG. 1: 1) Construct VH(anti-CD47)-CH1-H-CH2-CH3-(G4S)₄-VL(anti-HER2)-CL (SEQ ID NO:91), encoding the following elements: anti-CD47 heavy chain variable domain followed by human heavy chain constant domains 1-3 followed by a (G4S)₄ linker and anti-HER2 light chain variable domain followed by human kappa light chain constant domain. 2) Construct VL(anti-CD47)-CL (SEQ ID NO:74), encoding the following elements: anti-CD47 light chain variable domain followed by human kappa light chain constant domain. 3) Construct VH(anti-HER2)-CH1-H (SEQ ID NO:92), encoding the following elements: anti-HER2 heavy chain variable domain followed by human heavy chain constant domain 1 followed by the hinge region EPKSC (SEQ ID NO:10). The corresponding amino acid sequences for these three constructs are shown in SEQ ID NO:93, SEQ ID NO:76, and SEQ ID NO:94 respectively.

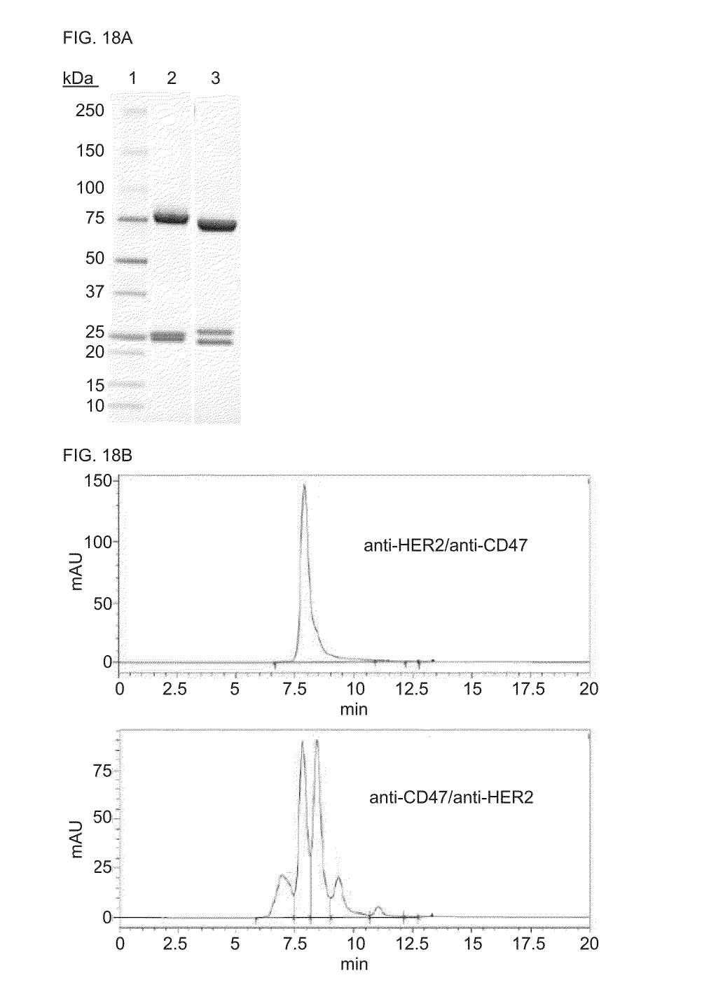

Each set of the three vectors was co-transfected transiently into Expi293 cells using Expi293fectin (Life Technologies, Grand Island, N.Y.) for expression of anti-HER2/anti-CD47 and anti-CD47/anti-HER2. The two TetBiAbs were purified in a single step by protein A affinity chromatography. Expression of the three polypeptides and assembly of the full hexameric molecule were confirmed on sodium dodecyl sulfate-polyacrylamide gel electrophoresis (SDS-PAGE) and size exclusion chromatography (SEC). For SDS-PAGE, the purified TetBiAbs samples were reduced with DTT and run on NuPAGE MES 4-12% Gel, 200V for 35 min, followed by Coomassie staining. The three major bands on the gel had the expected molecular weights (MW) and the correct stoichiometric ratio with >95% purity (FIG. 18A). In FIG. 18A, lane 1 shows the molecular weight (MW) marker, lane 2 shows the expected MW (74, 23, 23 kDa) and the correct stoichiometric ratio (1:1:1) of the three polypeptides of anti-HER2/anti-CD47, and lane 3 shows the expected MW (74, 24, 23 kDa) and the correct stoichiometric ratio (1:1:1) of the three polypeptides of anti-CD47/anti-HER2. For SEC, the purified TetBiAbs samples were analyzed on a TSK-GEL Super SW3000 SEC column 4.6×300 mm (Tosoh Biosciences, Tokyo, Japan) that was equilibrated with 50 mM sodium phosphate, 400 mM sodium perchlorate, pH 6.3+0.1 and 38+2.0 mS/cm². Size exclusion chromatography showed a peak at the expected MW of about 250 kDa for both the monomeric anti-HER2/anti-CD47 and anti-CD47/anti-HER2 (FIG. 18B).

In addition, a number of controls were generated to compare or optimize the TetBiAb format. These include anti-HER2 in a standard monoclonal antibody format (anti-HER2 IgG1) and anti-CD47 in a standard monoclonal antibody format (anti-CD47 IgG1).

9B) Binding of TetBiAbs to Antigens

The ability of anti-HER2/anti-CD47 and anti-CD47/anti-HER2 to retain binding to CD47 was measured by ELISA. Human CD47 was coated on 96 well plates overnight at 4° C. After washing with PBST, the wells were blocked with PBST+2% BSA for 1 hr at room temperature. After washing with PBST, varying concentrations of antibodies diluted in PBST+2% BSA were added to the wells and incubated for 1 hr at room temperature. After washing with PBST, HRP-conjugated Goat anti-Human IgG Fcγ (Jackson ImmunoResearch, West Grove, Pa.) diluted 1:10000 in PBST+2% BSA was added to the wells and incubated for 1 hr at room temperature. The bound antibodies were visualized with HRP substrate, 3,3′,5,5′-tetramethylbenzidine (TMB). The plates were measured for absorbance at 450 nm.

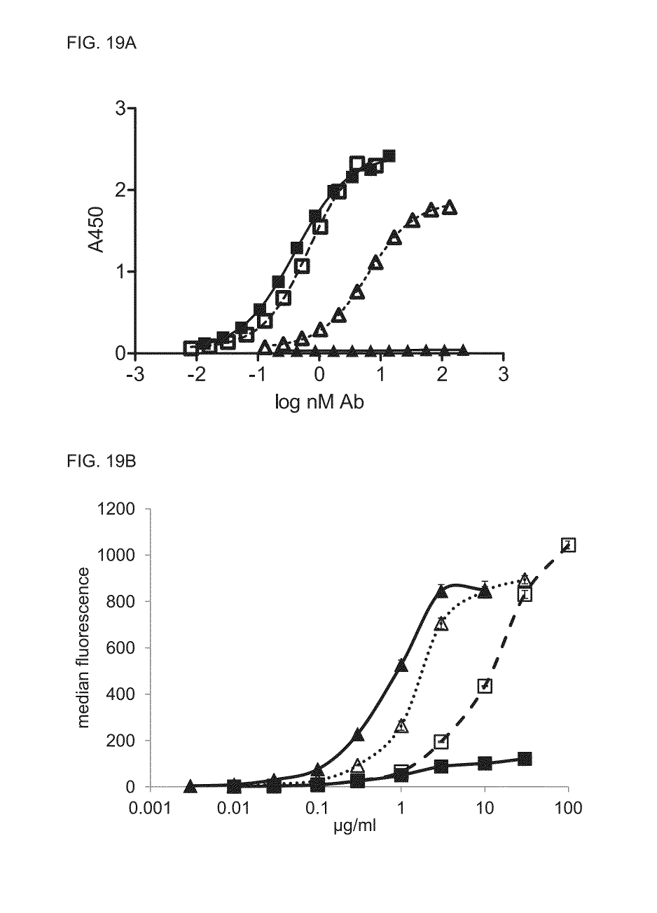

The results show that anti-CD47/anti-HER2 retains binding to CD47, similar to anti-CD47. Anti-HER2/anti-CD47 also retains binding to CD47, although it does not bind as well as anti-CD47 (FIG. 19A).

The ability of anti-HER2/anti-CD47 and anti-CD47/anti-HER2 to retain binding to HER2 on the cell surface was measured on human SK-BR-3 mammary gland/breast adenocarcinoma cells that overexpress HER2. 1×10⁵ SK-BR-3 cells per well were incubated with varying concentrations of anti-HER2/anti-CD47, anti-CD47/anti-HER2, anti-HER2, and anti-CD47 diluted in PBS+1% FBS in a 96 well plate for 60 min on ice. After washing with PBS+1% FBS, cells were incubated with FITC F(ab′)2 goat Anti-Human IgG, Fcγ (Jackson ImmunoResearch, West Grove, Pa.), diluted 1:200 in PBS+1% FBS for 60 min on ice. After washing again, cells were fixed with 1% formaldehyde in PBS. Cells were analyzed by flow cytometry (MACSQuant, Miltenyi Biotec, Cologne, Germany).

The results show that anti-HER2/anti-CD47 retains binding to SK-BR-3 cells, which express Her2, similar to anti-HER2. Anti-CD47/anti-HER2 also retains binding to HER2, although it does not bind as well as anti-HER2. Anti-CD47 does not bind to SK-BR-3 cells because CD47 is not expressed on SK-BR-3 cells (FIG. 19B).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is indicated by the appended claims rather than by the foregoing description, and all changes that come within the meaning and range of equivalency of the claims are intended to be embraced therein.

1. (canceled)

2. A tetravalent bispecific antibody (TetBiAb) comprising:
a first polypeptide comprising an antibody heavy chain of a first antibody, wherein said heavy chain contains a variable domain and constant domains of said first antibody, said heavy chain being linked, directly or indirectly, at its C-terminus to the N-terminus of an antibody light chain of a second antibody, wherein said light chain contains a variable and constant domain of said second antibody,
a second polypeptide comprising an antibody light chain of said first antibody, wherein said light chain of the first antibody contains a variable and constant domain, and
a third polypeptide comprising a Fab heavy chain of said second antibody, said third polypeptide lacking heavy chain constant domains CH2 and CH3,
wherein said first and second antibodies have different binding specificities, and
wherein said second polypeptide and cognate Fab heavy chain domains of said first polypeptide form a heterodimer, providing the binding specificity of said first antibody, and
wherein said third polypeptide and cognate light chain domains of the first polypeptide form a heterodimer, providing the binding specificity of said second antibody.

3. The tetravalent bispecific antibody (TetBiAb) of claim 2, wherein the constant domains of said first antibody are IgG constant domains.

4. The tetravalent bispecific antibody (TetBiAb) of claim 2, wherein said first polypeptide further comprises a linker linking the C-terminus of the heavy chain constant domain to the N-terminus of the light chain variable domain.

5-7. (canceled)

8. The tetravalent bispecific antibody (TetBiAb) of claim 2, wherein said third polypeptide further comprises a hinge region comprising the amino acid sequence of SEQ ID NO:10.

9. The tetravalent bispecific antibody of claim 2, wherein the binding specificities of the TetBiAb are to two different target antigens, wherein the first and second antigens are present on different cell types.

10. The tetravalent bispecific antibody of claim 2, wherein the binding specificities of the TetBiAb are to two different target antigens, wherein the first and second antigens are present on the same cell type.

11. The tetravalent bispecific antibody of claim 2, wherein the binding specificities of the TetBiAb are to two different epitopes on the same target molecule.

12. The tetravalent bispecific antibody of claim 2, wherein the binding specificities of the TetBiAb are to two different target antigens, wherein the first antigen is present on the surface of a cell and the second antigen is on a soluble factor.

13. (canceled)

14. The tetravalent bispecific antibody of claim 9, wherein the binding specificities of the TetBiAb are to a first target antigen on a tumor cell and to a second target antigen on an immune cell.

15. The tetravalent bispecific antibody of claim 9, wherein the binding specificities of the TetBiAb are to a target antigen pair, said pair consisting of the group selected from EGFR in combination with CD16, and CD20 in combination with CD16.

16. The tetravalent bispecific antibody of claim 10, wherein the binding specificities of the TetBiAb are to two different target antigens, wherein the first and second antigens are present on a tumor cell.

17. The tetravalent bispecific antibody of claim 9, wherein the binding specificities of the TetBiAb are to a target antigen pair, said pair selected from the group consisting of CD20 in combination with CD47, and CD20 in combination with CD52.

18. (canceled)

19. The tetravalent bispecific antibody of claim 15, wherein the TetBiAb comprises polypeptide chains comprising the amino acid sequence of SEQ ID NO:43, SEQ ID NO:14, and SEQ ID NO:44, polypeptide chains comprising the amino acid sequence of SEQ ID NO:47, SEQ ID NO:48, and SEQ ID NO:18, or polypeptide chains comprising the amino acid sequence of SEQ ID NO:51, SEQ ID NO:28, and SEQ ID NO:52.

20-25. (canceled)

26. The tetravalent bispecific antibody of claim 17, wherein the TetBiAb comprises polypeptide chains comprising the amino acid sequence of SEQ ID NO:59, SEQ ID NO:28, and SEQ ID NO:60, or polypeptide chains comprising the amino acid sequence of SEQ ID NO:67, SEQ ID NO:28, and SEQ ID NO:68.

27. The tetravalent bispecific antibody of claim 26, wherein the TetBiAb comprises polypeptide chains comprising the amino acid sequence having at least 85% identity to the amino acid sequence of SEQ ID NO:59, SEQ ID NO:28, and SEQ ID NO:60.

28-29. (canceled)

30. A tetravalent bispecific antibody comprising:
a first polypeptide comprising an antibody light chain of a first antibody, wherein said light chain contains a variable and constant domains of said first antibody, said light chain being linked at its C-terminus to the N-terminus of an Fc region, wherein said Fc region contains at least the hinge and CH3 domains, linked, directly or indirectly, to the N-terminus of an antibody heavy chain of a second antibody, said heavy chain containing the variable and CH1 domains of said second antibody,
a second polypeptide, comprising a Fab heavy chain of said first antibody, said second polypeptide lacking heavy chain constant domains CH2 and CH3, and
a third polypeptide, comprising an antibody light chain of said second antibody, wherein said light chain of the second antibody contains the variable and constant domains,
wherein said first and second antibodies have different binding specificities, and
wherein said second polypeptide and cognate light chain domains of said first polypeptide form a heterodimer, providing the binding specificity of said first antibody, and
wherein said third polypeptide and cognate Fab heavy chain domains of said first polypeptide form a heterodimer, providing the binding specificity of said second antibody.

31. The tetravalent bispecific antibody of claim 30, wherein said first polypeptide further comprises a linker linking the C-terminus of said CH3 domain to the N-terminus of the heavy chain variable domain.

32-34. (canceled)

35. The tetravalent bispecific antibody (TetBiAb) of claim 30, wherein said first polypeptide further comprises a hinge region C-terminal to the heavy chain CH1 domain comprising the amino acid sequence of SEQ ID NO:10.

36. An isolated DNA molecule, comprising a DNA sequence encoding a heavy chain of a first antibody (VH(1)-CH1-hinge-CH2-CH3) genetically fused via an optional linker to a light chain of a second antibody (VL(2)-CL)

37. The isolated DNA molecule of claim 36, further comprising a DNA sequence selected from (i) a sequence encoding light chain of the first antibody (VL(1)-CL) and (ii) a sequence encoding a Fab heavy chain of the second antibody (VH(2)-CH1).

38. A nucleic acid encoding a tetravalent bispecific antibody comprising the first, the second and the third polypeptides of the tetravalent antibody of claim 2.

39. A nucleic acid encoding a tetravalent bispecific antibody comprising the first, the second and the third polypeptides of the tetravalent antibody of claim 30.

40. A host cell comprising the nucleic acid of claim 38.

41. A method of making a tetravalent bispecific antibody comprising culturing the host cell of claim 38 under conditions suitable for the expression of the tetravalent bispecific antibody, and recovering the tetravalent bispecific antibody.

42. A pharmaceutical formulation comprising the tetravalent bispecific antibody of claim 15 and a pharmaceutically acceptable carrier.

43. A method of treating an individual having cancer comprising administering to the individual an effective amount of the tetravalent bispecific antibody of claim 15.

44. A host cell comprising the nucleic acid of claim 36.

45. A host cell comprising the nucleic acid of claim 39.
